SEQUENCE LISTING (12 sequences)

SEQ ID NO: 1 - protein, 43 aa, Artificial Sequence
  Lunasin peptide 1-43
SKWQHQQDSCRKQKQGVNLTPCEKHIMEKIQGRGDDDDDDDDD

SEQ ID NO: 2 - protein, 42 aa, Artificial Sequence
  Lunasin peptide 1-42
SKWQHQQDSCRKQKQGVNLTPCEKHIMEKIQGRGDDDDDDDD

SEQ ID NO: 3 - protein, 41 aa, Artificial Sequence
  Lunasin peptide 1-41
SKWQHQQDSCRKQKQGVNLTPCEKHIMEKIQGRGDDDDDDD

SEQ ID NO: 4 - protein, 40 aa, Artificial Sequence
  Lunasin peptide 1-40
SKWQHQQDSCRKQKQGVNLTPCEKHIMEKIQGRGDDDDDD

SEQ ID NO: 5 - protein, 39 aa, Artificial Sequence
  Lunasin peptide 1-39
SKWQHQQDSCRKQKQGVNLTPCEKHIMEKIQGRGDDDDD

SEQ ID NO: 6 - protein, 38 aa, Artificial Sequence
  Lunasin peptide 1-38
SKWQHQQDSCRKQKQGVNLTPCEKHIMEKIQGRGDDDD

SEQ ID NO: 7 - protein, 22 aa, Artificial Sequence
  Lunasin peptide 22 to 43
CEKHIMEKIQGRGDDDDDDDDD

SEQ ID NO: 8 - protein, 21 aa, Artificial Sequence
  Lunasin peptide 22-42
CEKHIMEKIQGRGDDDDDDDD

SEQ ID NO: 9 - protein, 20 aa, Artificial Sequence
  Lunasin peptide 22-41
CEKHIMEKIQGRGDDDDDDD

SEQ ID NO: 10 - protein, 19 aa, Artificial Sequence
  Lunasin peptide 22-40
CEKHIMEKIQGRGDDDDDD

SEQ ID NO: 11 - protein, 18 aa, Artificial Sequence
  Lunasin peptide 22-39
CEKHIMEKIQGRGDDDDD

SEQ ID NO: 12 - protein, 17 aa, Artificial Sequence
  Lunasin peptide 22-38
CEKHIMEKIQGRGDDDD

(19) United States
(12) Patent Application Publication
(10) Pub. No.: US 2018/0243357 A1
(43) Pub. Date: Aug. 30, 2018

(54) COMBINED COMPOSITIONS FOR CONTROLLING BLOOD SUGAR LEVELS, HEPATOPROTECTION, AND FOR PREVENTION AND TREATMENT OF RELATED MEDICAL CONDITIONS

(71) Applicant: NATURAL SHIELD ISRAEL 2016 LTD, kfar Tavor (IL)

(72) Inventor: Yaron ILAN, Kfar Tavor (IL)

(73) Assignee: NATURAL SHIELD ISRAEL 2016 LTD, Kfar Tavor (IL)

(21) Appl. No.: 15/757,583

(22) PCT Filed: Sep. 1, 2016
(86) PCT No.: PCT/IL2016/050959

§ 371 Date: Mar. 5, 2018

Related U.S. Application Data
(60) Provisional application No. 62/213,718, filed on Sep. 3, 2015.

(51) Int. Cl. A61K 36/185; A23L 33/11; A23L 33/18; A23L 33/105; A23L 33/00; A23L 2/52; A61K 31/575; A61K 38/02; A61P 1/16; A61P 3/10

(52) U.S. Cl. CPC A61K 36/185; A23L 33/11; A23L 33/18; A23L 33/105; A23L 33/40; A23V 2002/00; A61K 31/575; A61K 38/02; A61P 1/16; A61P 3/10; A23L 2/52

(57) ABSTRACT

The invention relates to compositions and methods using various combinations of at least one of phytosterols, lunasin peptide and plant extracts, optionally in combination with at least one of, soy extracts, beta-glycolipides and different adjuvants such as, castor oil, polyethylene glycol, or beta-cyclo dextrin, for controlling blood sugar levels, altered insulin resistance and/or hepatic function in a subject, treating an immune related disorder, treating liver damage and restoring liver function, treating, preventing, ameliorating, reducing or delaying the onset of acute or chronic toxic effect of a drug, and for enhancing the therapeutic effect of a therapeutic agent in a subject.

### FIELD OF INVENTION

The present invention relates to combined therapeutic compositions, beverage and food supplements with enhanced capability to control blood sugar levels, altered insulin resistance, and protect and restore liver function. Compositions of the present invention are particularly applicable to a range of disorders, including pre-diabetes, diabetes and related conditions.

### BACKGROUND REFERENCES

1. Rippe J M, Angelopoulos T J. Sugars, obesity, and cardiovascular disease: results from recent randomized control trials. Eur J Nutr. 2016 Jul. 14

2. Hamel C, Do sugar-sweetened beverages cause adverse health outcomes in adults? A systematic review protocol. Syst Rev. 2014 Sep. 23; 3:108.

3. Ludwig D S, et al. Relation between consumption of sugar-sweetened drinks and childhood obesity: a prospective, observational analysis. The Lancet, Volume 357, Issue 9255, pages 505-508 (2001).

4. Schulze M B, et al. Sugar-sweetened beverages, weight gain, and incidence of type 2 diabetes in young and middle-aged women. JAMA, Volume 292, Issue 8, pages 927-934 (2004).

5. Dhingra R et al. Soft drink consumption and risk of developing cardiometabolic risk factors and the metabolic syndrome in middle-aged adults in the community. Circulation, Volume 116, Issue 5, pages 480-488 (2007).

6. Rehm J. A systematic review of the epidemiology of unrecorded alcohol consumption and the chemical composition of unrecorded alcohol. Addiction. 2014 June; 109(6):880-93.

7. Swift R and Davidson D. Alcohol hangover: mechanisms and mediators. Alcohol Health & Research World, Volume 22, Issue 1, pages 54-60 (1998)

8. Menon K V et al. Pathogenesis, diagnosis, and treatment of alcoholic liver disease. Mayo Clinic Proceedings, Volume 76, Issue 10, pages 1021-1029 (2001).

9. O'Shea R S et al. Alcoholic liver disease: AASLD Practice Guidelines. Hepatology, Volume 51, Issue 1, pages 307-328 (2010).

10. Morgan M Y. The prognosis and outcome of alcoholic liver disease. Alcohol and Alcoholism, Volume 2 (Suppl), pages 335-343 (1994).

11. Galvez A F. et al., A novel methionine-rich protein from soybean cotyledon: cloning and characterization of cDNA Plant Physiol. 114:1567-9 (1997).

12. WO 2012/017435.

13. WO 2016/030876

14. WO 2015/159290.

15. Erickson S K. Nonalcoholic fatty liver disease. J Lipid Res, Volume 50 (Suppl), pages S412-S416 (2009).

### BACKGROUND OF THE INVENTION

Stability of the level of blood glucose (or blood sugar) is the basic prerequisite for maintenance of controlled influx and availability of glucose to the cells. Glucose, being the preferential source of energy in virtually all body cells, is essential for normal function of all body systems, which is why blood glucose levels are tightly regulated as a part of metabolic homeostasis governed by the feedback of insulin/glucagon produced in the pancreas.

In all vertebrates, regardless of large fluctuations in physical activity and food intake, blood sugar levels are held within very narrow limits. In humans, the normal blood glucose levels (tested while fasting) for non-diabetics, are on average between 70-100 milligrams per deciliter (mg/dL). Blood glucose levels outside the normal range, i.e. persistent hyper- or hypo-glycemia, may be an indicator of a number of medical conditions. Diabetes mellitus characterized by persistent hyperglycemia is the most prominent disease related to failure of blood sugar regulation. Intake of alcohol causes an initial surge in blood sugar, and later tends to cause hypoglycemia.

Apart from issues of lifestyle and self-esteem, controlling blood sugar levels and maintaining a healthy weight are vital in order to lower the risk of diseases such as type 2 diabetes (the most common adult form of diabetes resulting from insulin resistance), morbid obesity, heart disease, liver disease and cancer. Consequences of chronic alcohol consumption are numerous, apart from risks of injuries sustained in car accidents and liver cirrhosis, there are also risks of anemia, cardiovascular disease, cancer and distinct neurologic and psychiatric disorders. Sugar enriched foods, particularly soft drinks, and alcohol are considered among major health hazards produced by a modern way of living. The Gallup's Annual Consumption Habits Poll conducted in Jul. 9-12, 2012 in the US, for example, indicated that about half of all Americans, 48%, consume on average at least one glass of a soda per day and 66%—over four alcoholic drinks per week [1].

According to the American Heart Association, soft drinks and other sugar-sweetened beverages (SSBs) are the primary source of added sugars in Americans' diets; their increased consumption has been associated with rising obesity rates. Consumption of SSBs has increased 500% in the past fifty years and is now the single largest category of caloric intake in children, about 10-15% of the total daily calorie intake [2]. The rising prevalence of obesity in children has been linked, in part, to the consumption of SSBs [3]. Consumption of excessive calories and large amounts of rapidly absorbable sugars through SSBs was recognized as one of significant contributors to weight gain and incidence of type 2 diabetes in American women between 1991 to 1999 [4]. In fact, individuals consuming one or more SSB per day have higher odds for developing metabolic syndrome (odds ratio OR=1.48), obesity (OR=1.31), increased waist circumference (OR=1.30), impaired fasting glucose (OR=1.25), higher blood pressure (OR=1.18), hyper-triglyceridemia (OR=1.25), and low high-density lipoprotein cholesterol (OR=1.32) [5]. A recent research by the Harvard School of Public Health summarizing data for Global Burden of Disease for 2010, suggested that SSBs were directly responsible for 133,000 deaths from diabetes, 44,000 deaths from cardiovascular disease and 6,000 deaths from cancer worldwide and for a total of 25,000 deaths in the US alone [6].

Concerns with regard to excessive sugar and alcohol consumption imposed by the modern life style are clear. Public health policy makers and professionals are currently conducting a number of policies to control consumption, including taxation and legislation. The food and beverage industry is increasingly replacing sugary products with sugar-free or artificially sweetened versions. There is however apparent shortage of candidate food additives, natural or synthetic, having potential to counter-balance the negative effects of both, excess sugar and alcohol. Two food additives that are being presently considered as blood sugar busting components are vinegar and cinnamon

According to the Centers for Disease Control (CDC) statistics for 2006-2010, there are annually 88,000 deaths attributable to excessive alcohol consumption in the US alone, making alcohol the third leading lifestyle-related cause of death in the nation. In US 2006, for example, there were more than 1.2 million emergency room visits and 2 7 million physician office visits due to excessive drinking [6]. Over time, excessive alcohol use can lead to the development of chronic diseases, including liver disease, alcoholic hepatitis and cirrhosis, the latter are among the leading causes of deaths in the US. Long-term health risks also include, but are not limited to, neurological impairments, cardiovascular problems, and psychiatric and social problems.

Several alterations in the metabolic state of the liver and other organs occur in response to the presence of alcohol (ethanol) in the body and can result in low blood sugar levels (hypoglycemia) [7]. Alcohol metabolism leads to a fatty liver and buildup of an intermediate metabolic product, lactic acid, in body fluids (lactic acidosis). Both of these effects can inhibit glucose production. Alcohol-induced hypoglycemia generally occurs after prolonged alcohol consumption coupled with poor nutritional intake, which not only decreases glucose production but also exhaust the reserves of glucose stored in the liver in the form of glycogen, thereby leading to hypoglycemia. Because glucose is the primary energy source of the brain, hypoglycemia can contribute to hangover symptoms such as fatigue, weakness, and mood disturbances. Diabetics are particularly sensitive to the alcohol-induced alterations in blood glucose.

Excessive alcohol consumption is the major cause of liver disease; 15-20% of chronic heavy drinkers develop hepatitis or cirrhosis that can occur concomitantly or in succession. While genetic factors may contribute both to alcoholism and to alcoholic liver disease, malnutrition, particularly vitamin A and E deficiencies, can worsen alcohol-induced liver damage by preventing hepatocyte regeneration [8]. Women are twice as susceptible to alcohol-related liver disease, and may develop alcoholic liver disease with shorter durations and doses of chronic consumption. Alcoholic liver disease evolves as a result of secretion of pro-inflammatory cytokines, oxidative stress, lipid peroxidation and acetaldehyde toxicity ensuing in response to alcohol consumption. These factors cause inflammation, apoptosis and eventually fibrosis of liver cells [9].

Alcoholic liver disease evolves from fatty change through alcoholic hepatitis to alcoholic cirrhosis. Its development is associated with an excess mortality both in relation to the presence of liver disease and to other complications of alcohol abuse. In the majority of patients fatty liver is a benign lesion, which will reverse completely following abstinence from alcohol. Continued drinking is associated with the eventual development of cirrhosis in approximately 20% of individuals. Alcoholic hepatitis is a precirrhotic lesion, progression to cirrhosis is observed more commonly in women, in individuals with severe disease and in those who continue to drink. Thirty-day mortality rates of less than 20% are observed in patients with mild to moderate disease but exceed 40% in individuals with severe liver injury. Survival is significantly reduced in women and in the elderly and is adversely affected by the presence of severe liver injury, evolution to cirrhosis and continued drinking Two-thirds of patients with alcoholic cirrhosis present with decompensated disease; 15% will develop hepatocellular carcinoma. Survival is adversely affected by the presence of decompensated disease, superimposed alcoholic hepatitis, continued drinking and the development of hepatocellular carcinoma [10].

Lunasin is the major component of the Bowman-Birk Protease Inhibitor (BBIC) derived from soybean, a known cancer preventive substance. Lunasin was initially identified in the soybean cotyledon when a cDNA encoding a post-translationally processed 2S albumin (Gm2S-1) was cloned from mid-maturation soybean seed. This 43 amino acid peptide exhibits in cells mitotic arrests leading to cell death, and is therefore applicable for treating cancer [11].

Phytosterols and phytostanols, also referred to as plant sterols and stanols, are common plant and vegetable constituents and are therefore normal constituents of the human diet. Plant sterol consumption have been show as connected with reduction of LDL-Cholesterol and is therefore suggested for treating cardiovascular disorders.

Moringa is a plant native to the sub-Himalayan areas of India, Pakistan, Bangladesh, and Afghanistan and is also grown in many other areas of the world. Different extracts and preparations of this plant have been implicated in various pathologic conditions including anemia, arthritis, asthma, cancer, constipation, diabetes, diarrhea, epilepsy, intestinal ulcers, intestinal spasms, headache; heart problems, high blood pressure, kidney stones, fluid retention; thyroid disorders and bacterial, fungal, viral and parasitic infections.

WO 2012/017435, WO 2016/030876 and WO 2015/159290 [12, 13, 14], are previous publications of the present inventor that describe compositions, methods and uses of different soybean extracts and combinations thereof, for the treatment of hepatic disorders, drug induced hepatic injury and related metabolic disorders.

Thus, there is a major need for therapeutic compounds, food supplements, food additives, medical foods, botanical drugs and safe drugs assisting in control of blood sugar levels and thereby facilitating prevention and amelioration of related disorders.

### SUMMARY OF INVENTION

A first aspect of the invention relates to compositions for controlling blood sugar levels, altered insulin resistance and/or hepatic function, treating various metabolic and immunological conditions, including pre-diabetes, diabetes and inflammatory disorders. The compositions of the invention are applicable in treating liver damage, restoring liver function, treating, preventing, ameliorating, reducing or delaying the onset of acute or chronic toxic effect of a drug, enhancing the therapeutic effect of a therapeutic agent and for treating various related malignancies. More specifically, the compositions of the invention may comprise as an active ingredient:

I. at least one of:

(a) at least one natural or synthetic sterol or a derivative or a mixture thereof;

(b) lunasin peptide or a derivative thereof; and

(c) at least one extract of a plant from the genus Moringa.

In some embodiments, the compositions of the invention may optionally further comprise at least one of:

II. at least one of:

(d) at least one soy extract (SE) or any fraction thereof;

(e) at least one natural or synthetic beta-glycolipid or any derivative thereof;

(f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

A further aspect of the invention relates to a method for controlling blood sugar levels, altered insulin resistance and/or hepatic function in a subject, treating various metabolic and immunological conditions, including diabetes and inflammatory disorders, further treating various related malignancies, still further treating liver damage and restoring liver function, treating, preventing, ameliorating, reducing or delaying the onset of acute or chronic toxic effect of a drug, and enhancing the therapeutic effect of a therapeutic agent. The method of the invention comprises the step of providing to a subject at least one of:

I. at least one of:

(a) at least one natural or synthetic sterol or a derivative or a mixture thereof;

(b) lunasin peptide or a derivative thereof; and

(c) at least one extract of a plant from the genus Moringa;

In some embodiments, the compositions of the invention may optionally further comprise at least one of:

II. at least one of:

(d) at least one soy extract (SE) or any fraction thereof;

(e) at least one natural or synthetic beta-glycolipid or any derivative thereof;

(f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

Still further, the invention provides a soft or an alcoholic beverage or food comprising

I. at least one of:

(a) at least one natural or synthetic sterol or a derivative or a mixture thereof;

(b) lunasin peptide or a derivative thereof; and

(c) at least one extract of a plant from the genus Moringa;

It should be noted that the soft or an alcoholic beverage or food according to the invention further comprise and at least one of:

II. at least one of:

(d) at least one soy extract (SE) or any fraction thereof;

(e) at least one natural or synthetic beta-glycolipid or any derivative thereof;

(f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

In yet another aspect, the invention provides a combined compositions comprising as an active ingredient the above-described constituents, specifically, any combinations of the constituents defined in (I) and (II).

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A-1C: The Effect of CardioAid and Lunasin on the Immune System

FIG. 1A: A bar graph showing the percentage of CD4 positive cells in splenocytes lymphocytes obtained from mice fed on a high fat diet (control) or from mice fed on a high fat diet and administered with CardioAid or Lunasin.

FIG. 1B: A bar graph showing the percentage of CD8 positive cells in splenocytes lymphocytes obtained from mice fed on a high fat diet (control) or from mice fed on a high fat diet and administered with CardioAid or Lunasin.

FIG. 1C: A bar graph showing the CD4/CD8 ratio in splenocytes lymphocytes obtained from mice fed on a high fat diet (control) or from mice fed on a high fat diet and administered with CardioAid or Lunasin.

FIG. 2A-2B: The Effect of CardioAid and Lunasin on Serum Cytokines

FIG. 2A: A bar graph showing the level of IL-la in serum obtained from mice fed on a high fat diet (control) or from mice fed on a high fat diet and administered with CardioAid or Lunasin.

FIG. 2B: A bar graph showing the level of TGFβ in serum obtained from mice fed on a high fat diet (control) or from mice fed on a high fat diet and administered with CardioAid or Lunasin.

FIG. 3A-3B: The Effect of CardioAid and Lunasin on Liver Enzymes Secretion

FIG. 3A: A bar graph showing the level of Alanine aminotransferase (ALT) and Aspartate aminotransferase (AST) in blood obtained from mice fed on a high fat diet (control) or from mice fed on a high fat diet and administered with CardioAid or Lunasin.

FIG. 3B: A bar graph showing the level of gammaglutamyl transferase (GGT) in blood obtained from mice fed on a high fat diet (control) or from mice fed on a high fat diet and administered with CardioAid or Lunasin.

FIG. 4A-4B: The Effect of CardioAid and Lunasin on Liver Histology

FIG. 4A: A bar graph showing the NAS score of liver sections obtained from mice fed on a high fat diet (control) or from mice fed on a high fat diet and administered with CardioAid or Lunasin.

FIG. 4B: A bar graph showing the Ballooning score of liver sections obtained from mice fed on a high fat diet (control) or from mice fed on a high fat diet and administered with CardioAid or Lunasin.

FIG. 5: The Effect of CardioAid and Lunasin on Total Body Fat

A bar graph showing the percentage of total body fat evaluated using EchoMRI in mice fed on a high fat diet (control) or from mice fed on a high fat diet and administered with CardioAid or Lunasin.

FIG. 6: The Effect of CardioAid and Lunasin on Hepatic Triglycerides

A bar graph showing the level of triglycerides (TG) in the liver of mice fed on a high fat diet (control) or from mice fed on a high fat diet and administered with CardioAid or Lunasin.

FIG. 7: The Effect of CardioAid and Lunasin on Serum Glucose Levels

A graph showing the change in serum glucose levels with time in mice fed on a high fat diet (control) or from mice fed on a high fat diet and administered with CardioAid or Lunasin.

FIG. 8: The Effect of CardioAid Added to a Beverage on Glucose Levels

A graph showing the change in serum glucose levels with time in mice fed with a chocolate drink or with a chocolate drink supplemented with CardioAid.

FIG. 9: The Effect of Moringa and OS or M1 Soy Extracts on Immune Mediated Liver Damage

A bar graph showing ALT serum levels in mice treated with Con A to induce immune mediated hepatitis that were treated with Moringa extract or with a combination of Moringa with OS or with M1 extracts.

### DETAILED DESCRIPTION OF THE INVENTION

The present invention stems from current findings by the inventors showing that various combinations of phytosterols, lunasin peptide and plant extracts, specifically, Moringa extracts possess surprising properties revealed in protecting an organism against altered blood glucose levels, altered insulin resistance and hepatic dysfunction. Present experiments may further suggest that these properties could be enhanced by combining the above combinations with soybean extracts, beta-glycolipides and/or adjuvants such as polyethoxylated castor, polyethylene glycol or beta-cyclo dextrin.

More specifically, the inventors presently show that supplementation of specific combinations comprising phytosterols, plant extracts, soy extracts and lunasin peptide, with or without adjuvants, to foods or beverages with high sugar and/or alcohol content have beneficial long-term effects on clinical indices of glucose tolerance, and thereby control alterations in insulin resistance. Furthermore, when these combinations are administered to a mammal subject to hepatic insult, either immune- or drug-mediated, these combinations have beneficial restorative effects on specific biochemical indices of liver function.

These insights imply that compositions of the present invention, namely compositions comprising certain combinations of phytosterols, plant extracts, soy extracts and lunasin peptide, with or without adjuvants and/or soy extracts, may have therapeutic applications to a range of clinical conditions associated with altered blood sugar levels, metabolic and/or hepatic dysfunction, including pre-diabetes, diabetes, fatty liver disease and various types of hepatitis and related conditions, and further to various inflammatory disorders and malignancies.

It is further conceived that compositions of the present invention may serve basis for the development of new therapeutic compounds for treatment of hepatopathologies in a wide range of clinical contexts, including alleviation of immune-induced, drug-induced or alcohol-induced liver damage, as well as cirrhosis and/or hepatic failure ensuing from infections, cancer, alcoholic steatohepatitis, non-alcoholic steatohepatitis (NASH or NAFLD) and other chronic liver diseases.

In this context, it is contemplated that compositions of the present invention may be applicable not only for treating, preventing, ameliorating an existing liver damage and improving liver function, but also that said compositions when administered concomitantly or prior to other drugs may enhance their therapeutic effects, and increase overall benefits of therapeutic regimens.

By their nature, compositions of the present inventions, consisting of combinations of phytosterols, lunasin peptide/s, plant extracts and optionally, soy extracts, with or without adjuvants, should be safe and lacking major adverse effects. It is thus contemplated that these compositions may be applicable not only in a secondary prevention of already existing clinical disorders but also in a primary prevention of risks or pre-clinical conditions in a normal population.

It is thus contemplated that compositions of the present invention may be used as “bouncers” in preventing the development of pre-clinical conditions ensuing from exposure to foods and beverages with high sugar and/or alcohol content. In this context, compositions of the invention, rather than being used as therapeutic agents, are used as food meant to normalize risks ensuing from modern lifestyle to which are subjected normal individuals. Such medical foods, food supplements or food additives, especially in form of add-on to sugar sweetened and/or alcoholic beverages, may be especially beneficial for preventing common conditions, such as weight gain, alcohol intoxication and risk of cardiovascular pathology, and also more severe presentations, such as obesity and alcohol withdrawal syndrome.

Thus, in its first aspect the present invention provides compositions for use in at least one of, a method for controlling blood sugar levels, altered insulin resistance and/or hepatic function in a subject, a method for the treatment of an immune related disorder, a method of treating liver damage and restoring liver function, a method for treating, preventing, ameliorating, reducing or delaying the onset of acute or chronic toxic effect of a drug, and a method for enhancing and augmenting the therapeutic effect of a therapeutic agent in a subject treated with said agent. These compositions may comprise as an active ingredient:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the compositions of the invention may optionally further comprise at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

Specific combinations of the above components of the compositions of the invention, which may applicable to specific embodiments of methods for treating specific disorders, are further contemplated. More specifically, it is meant that specific combinations of compositions of the invention may include, but not limited to, the following examples.

In some specific embodiments, the compositions of the invention may comprise as an active ingredient sterol or a derivative or a mixture thereof. In more specific embodiments, such sterols may be at least one plant stanols and sterol and any esters or mixtures thereof.

The term ‘sterol’, in its broadest sense, refers to steroid alcohols that are a subgroup of steroids. In specific embodiments, it refers to the naturally occurring sterols obtained from plants (i.e. phytosterols), animals, and fungi. Among phytosterols and their derivatives, of particular relevance to the present invention is CardioAid™ (or CardioAid®) acknowledged as GRAS (Generally Recognized As Safe) chemical or additive to food by the American Food and Drug Administration (FDA). CardioAid is commercially available from Archer Daniels Midland (ADM). A common preparation of CardioAid may include, for example, about 50% beta-sitosterol, 25-30% campesterol, 20% stigmasterol, and up to 5% brassicasterol and 5% sitosterol, and other ingredients such as gelatin, rice flour, magnesium stearate.

In some specific embodiments, the sterol component of the compositions of the invention, or a sterol derivative or mixture of sterols, may be Cardioaid™ comprising at least one of CardioAid-S, CardioAid-XF, CardioAid-SWD and CardioAid-SF.

The term CardioAid in this context encompasses various preparations of CardioAid, all available from ADM, namely CardioAid-S, CardioAid-XF, CardioAid-SWD and CardioAid-SF. More specifically CardioAid-S is a creamy-white to pale-yellow paste produced by esterifying plant sterols with canola oil fatty acids. CardioAid-SWD is an off-white granular powder produced by esterifying vegetable oil sterols with food grade rapeseed oil fatty acids. CardioAid-SF is also creamy-white to pale-yellow paste are derived by esterifying sterols with canola oil fatty acids. CardioAid-XF is a non-esterified phytosterol product an off-white powder it is water-insoluble. Apart from their appearance and way of production, these various CardioAid preparations may differ in flavor and odor, and in melting point.

It should be however appreciated that the invention encompasses the use of any of these CardioAid preparations or any combinations thereof.

The term ‘lunasin’ herein refers to lunasin peptide that is found in small quantities in soybean seeds and soy-based foods, and also in some cereal grains. More specifically, lunasin is a unique 43-amino acid peptide containing 8 Asp (D) residues in its carboxyl end (bold) preceded by a cell adhesion motif Arg-Gly-Asp (RGD) (italics) and a predicted helix (underlined) with structural homology to a conserved region of chromatin-binding proteins [lunasin amino acid sequence:

S K W Q H Q Q D S C R K Q K Q G V N LT P C -
EKHIMEKIQG-RGD-DDDDDDDD,

as also denoted by SEQ ID NO. 1]. Lunasin was investigated as an anti-cancer agent, as over-expression of lunasin in the cells was related to mitotic arrests leading to cell death. The antimitotic effect of this peptide was attributed to binding of its poly-D carboxyl end to regions of hypoacetylated chromatin, such as that found in kinetochores in centromeres, thereby leading to abnormal formation of the kinetochore complex, non-attachment of microtubules to centromeres, and eventual mitotic arrest and cell death. It should be therefore appreciated that for treating the above-mentioned conditions the invention encompasses the use of any preparation of Lunasin peptide as well as of any fragments and derivatives therefore. In certain embodiments, the compositions of the invention may comprise as an active ingredient at least one lunasin peptide or any derivative thereof. In some specific embodiments, such lunasin peptide may comprise the amino acid sequence as denoted by SEQ ID NO. 1, or any derivatives or functional fragments thereof. In some specific embodiments lunasin peptide used by the compositions and method of the invention may comprise a partial sequence or fragments derived from the amino acid sequence of SEQ ID NO. 1. Non-limiting examples for lunasin fragments that may be applicable in the present invention include but are not limited to a peptide having amino acid residues 1 to 42 of SEQ ID NO. 1, such peptide is denoted by SEQ. ID. 2, a peptide having amino acid residues 1 to 41 (SEQ. ID. 3), a peptide having amino acid residues 1 to 40 (SEQ. ID. 4), a peptide having amino acid residues 1 to 39 (SEQ. ID. 5), a peptide having amino acid residues 1 to 38 (SEQ. ID. 6), a peptide having amino acid residues 22 to 43 (SEQ. ID. 7), a peptide having amino acid residues 22 to 42 (SEQ. ID. 8), a peptide having amino acid residues 22 to 41 (SEQ. ID. 9), a peptide having amino acid residues 22 to 40 (SEQ. ID. 10), a peptide having amino acid residues 22 to 39 (SEQ. ID. 11), and a peptide having amino acid residues 22 to 38 (SEQ. ID. 12), or any combinations thereof.

Still further, lunasin is sold as a branded ingredient (LunaRich, LunaRichX, Lunasin Reliv, Lunasin XP and Relive, all by Reliv International and previously Soy Labs LLC) available in capsules and as soy protein drinks.

Thus, in yet some other specific embodiments, the lunasin component of the compositions of the invention, or a derivative of lunasin peptide, may be any one of Lunasin Reliv, LunaRichX and Relive.

In some specific embodiments, the composition of the invention may comprise as an active ingredient at least one extract of Moringa. In some specific embodiments, the extract of the compositions of the invention may be an extract of Moringa oleifera.

Under Moringa plant is meant the sole genus in the flowering plant family Moringaceae, which is native to parts of Africa and Asia. This genus contains 13 species from tropical and subtropical climates that range in size from tiny herbs to massive trees. The most widely cultivated species is Moringa oleifera, a multipurpose tree native to the foothills of the Himalayas in northwestern India, and cultivated throughout the tropics. Moringa stenopetala, an African species, is also widely grown, but to a much lesser extent than Moringa oleifera.

Different parts of a Moringa plant contain a profile of important minerals and are a good source of protein, vitamins, beta-carotene, amino acids and various phenolics. Moringa provides a rich and rare combination of zeatin, quercetin, beta-sitosterol, caffeoylquinic acid and kaempferol.

Thus, under an extract of a Moringa plant is meant herein any substance or a mixture of substances extracted from any part of Moringa plant, using either enzymatic extracts, organic solvents or by hydrophilic solvents. In other words, the term extract encompasses substances obtained by using either organic solvents such as, for example, hexane, ethyl-acetate or isopropyl-alcohol, or by hydrophilic solvents such as water. The extracts may be dried after said extraction and may be further extracts by any extraction method, independently from previous extraction steps. Such steps may be repeated independently. Furthermore, other extraction techniques may be employed, non-limiting examples of which include chromatography, including size-exclusion, hydrophobic interaction, and anion and cation exchangers, differential centrifugation, differential precipitation (for example, using ammonium sulfate), differential filtration and dialysis.

As noted above, in some specific embodiments, the compositions of the invention may comprise as an active ingredient at least one of natural or synthetic plant sterols, lunasin peptide/s and extract of a plant from the genus Moringa or any combinations thereof. In yet some alternative specific embodiments, such compositions may further comprise additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof.

As indicated above, other particularly relevant combinations of the compositions of the invention in addition to at least one of natural or synthetic plant sterols, lunasin peptide/s and extract of a plant from the genus Moringa or any combinations thereof, may further include natural or synthetic beta-glycolipid. In some specific embodiments, as a natural or synthetic beta-glycolipid component the compositions of the invention may comprise any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof. In other words, the beta-glycolipid component of the invention may be any synthetic or natural beta-glycolipid or any derivative or combination thereof. Further, the beta-glycolipid of the invention may be selected from the group of glycosphingolipids, of a natural or non-natural source, with any number of carbons and double bonds and with any length of the lipid tail of the molecule. More specifically, the beta-glycolipid of the invention may be a glucosylceramide, a monosaccharide ceramide, a galatosylceremide, a lactosyl-ceramide, a gal-gal-glucosyl-ceramide, GM2 ganglioside, GM3 ganglioside, or globoside, or similar soy derived products.

In certain preferred embodiments of the invention, the above beta-glycolipid is a natural or synthetic beta-glucosylceramide (GC). The natural GC is the only glycosphingolipid common to plants, fungi and animals, in all of which it constitutes a major component of the outer layer of the plasma membrane. GC is considered to be the principal glycosphingolipid in plants. In animals, GC is a major constituent of skin lipids, where it is essential for lamellar body formation in the stratum corneum and to maintain the water permeability barrier of the skin. Lower levels of GC are found in cells of the spleen, erythrocytes, and nervous tissues, especially the neurons.

In yet some further specific embodiments, the compositions of the invention may comprise in addition to the least one of natural or synthetic plant sterols, lunasin peptide/s and extract of a plant from the genus Moringa or any combinations thereof described above, also at least one soy extract. Particularly relevant combinations of the compositions of the invention may include as SE component (i.e. soy extract) a soy derived polar and/or nonpolar fraction or any fraction thereof.

Under soy is meant any part of a plant belonging to the genus Glycine, including the two subgenera, Glycine and Soja. Seeds (also beans) or pollen of said plants are of particular applicability to the present invention. Further pertinent thereto, genetically modified soy, which may include, among others, glyphosate-tolerant or herbicide-tolerant soy that constitute now the majority of the commercial market (e.g. 93% in the US).

As noted above, the term extract refers to any substances obtained by extracting soy, particularly soybeans, using either enzymatic extracts, organic solvents or by hydrophilic solvents. Many extraction methods may be used for producing SE of the invention. For example, at least one of an aliphatic organic solvent and water, or supercritical carbon dioxide gas may be used as an extractant for extraction of phospholipids from soybeans, preferably a defatted soybean material. The aliphatic organic solvent is preferably a saturated hydrocarbon, an alcohol, a mixed solvent of saturated hydrocarbon and alcohol, or a mixed solvent of halogenated hydrocarbon and alcohol. It is preferable that the extract be at least one of hexane, ethanol, methanol, hydrous ethanol, isopropyl alcohol, acetonitrile and acetone.

Further, SE may be enriched with aromatic chromophore containing compounds including the isoflavones genistein, daidzein, formononetin and biochanin and/or their glycosides, and for administration it is generally provided in association with one or more pharmaceutically acceptable carriers, excipients, auxiliaries, and/or diluents. Other procedures for specifically enriching or removing soybean isoflavones include differential extraction with organic solvents, based on the differing solubility of aromatic chromophore containing compounds in certain organic solvents.

Apart from extracts derived from the soybean, other extracts may be derived from the solvent extraction of soy pollens into oil which contains tri- and di-glicerydes, free fatty acids and phosphatides, as well as extracts derived from aqueous-ethanol extraction left after the solvent extraction, which contains soy protein, isoflavones, sugars (oligo-, di-, mono-), and lipids (including phosphatides, phytosterols, saponins).

Thus, for the purpose of certain embodiments and methods of the invention, compositions of the invention may comprise any soy derived preparation extract.

As described in the art, SE may also incorporate enzymatic treatment of said soybeans, or other soy plant material, whether before, during or after mechanical disruption and/or chemical extraction of plants. Therefore, enzymatic treatment of the plant material is specifically contemplated herein. Enzymes used for said extraction include cellulase, hemicellulase, pectinase, protease and other carbohydrases. The use of enzymatic treatment may be carried out under various moisture and temperature conditions suitable for optimal enzyme activity as known in the art. When performing enzymatic treatment of the soy plant material during chemical extraction, it is appreciated that the solvent and conditions used must be compatible with the maintenance of adequate enzymatic activity, and care must be taken not to inhibit the enzyme activity or to denature it.

Of particular relevance to the present invention compositions comprising as an active ingredient a soy derived fraction which is either soy derived polar or non-polar fraction. Said polar and/or non-polar fractions, may be in particular embodiments soy extract fractions presently designated as M1 and OS respectively. These specific fractions of SE may be obtained by standard processing procedures for extracting soy oil and soy protein. When subjected to qualitative LC-MS and ¹H-, ³¹P-NMR analyses, M1 and OS fractions can be identified with characteristic chemical profiles, as further detailed below.

For example, the M1 (polar) fraction can be obtained by standard hydro-alcoholic extraction of defatted soy milk to food soy protein. Specific constituents of M1 and OS fractions may be identified using qualitative LC-MS, ¹H-NMR analyses. For LC-MS analysis, the M1 fraction is dissolved in DMSO and analyzed on C-18 reversed column and polar mobile phase consisting of water (modified with ammonium formate) and methanol. For the ¹H-NMR analysis—the M1 fraction is dissolved in different solvents. According to both analyses, M1 is characterized by typical phosphatidylcholine (PC) and phosphatidylinositol (PI) content, in declining order. According to more accurate ³¹P-NMR analysis, M1 is characterized by a highly heterogeneous content of phospholipids and phosphatides. M1 is predominantly enriched in phosphatidylcholine (PC) and phosphatidylinositol (PI). The MI fraction, which is derived from aqueous-ethanol extraction left after the solvent extraction as detailed above, contains isoflavones, sugars (oligo-, di-, mono-), and lipids (including—phosphatides, phytosterols, saponins).

For LC/MS analysis, the OS (non-polar) fraction is dissolved in chloroform and analyzed on reversed column C-18 and non-polar mobile phase consisting of methanol and ethyl acetate. According to LC/MS and NMR analyses, the OS fraction predominantly contains glycerides and phospholipids, in declining order. By ³¹P-NMR spectroscopy, OS is mainly enriched in phosphatidic acid (PA), phosphatidylethanolamine (PE) and phosphatidylcholine (PC). OS and M1 fractions are distinct by ratios of various phosphatides. The OS fraction, derived from the solvent extraction of soybeans into oil, contains tri- and di -glycerides, free fatty acids & phosphatides.

Knowing the specific constituents of M1 and OS fractions, it is conceived that certain compositions of the present invention may comprise not only natural but also synthetic M1 or OS fractions or any partial constituents thereof or any combination of said constituents.

More specific embodiments of compositions and methods of the invention relate to compositions comprising as an active ingredient at least one of natural or synthetic plant sterols, lunasin peptide/s and extract of a plant from the genus Moringa or any combinations thereof and in addition, the M1 fraction of a soy extract.

More specifically, in specific embodiments the compositions of the invention comprising at least one of natural or synthetic plant sterols, lunasin peptide/s and extract of a plant from the genus Moringa or any combinations thereof and a soy derived polar fraction (designated M1) may comprise at least one of phospholipids, phosphatides or a combination thereof.

In yet further specific embodiments, the above compositions may comprise phosphatides may comprise characteristic of the polar fraction M1, which are any one of phosphatidylcholine (PC), phosphatidylinositol (PI) or a combination thereof.

Yet in other specific embodiments and methods, compositions of the present invention may comprise at least one of natural or synthetic plant sterols, lunasin peptide/s and extract of a plant from the genus Moringa or any combinations thereof and in addition, a soy derived non-polar fraction (OS) comprising at least one of glycerides, and further phospholipids and phosphatides are any one of phosphatidic acid (PA), phosphatidylethanolamine (PE) and phosphatidylcholine (PC), which are characteristic of OS.

More specific embodiments relate to a composition according to the invention comprising as an active ingredient at least one of natural or synthetic plant sterols, lunasin peptide/s and extract of a plant from the genus Moringa or any combinations thereof and in addition, the OS soy extract.

Another specific embodiment relates to compositions comprising as an active ingredient a combination of the M1 and OS soy extract fractions.

In yet another particular embodiment, the composition of the invention may comprise as an active ingredient at least one of natural or synthetic plant sterols, lunasin peptide/s and extract of a plant from the genus Moringa or any combinations thereof, and at least one polyethoxylated castor oil or any derivative thereof. Still further specific embodiments of the invention relate to compositions comprising as an active ingredient a combination of at least one of natural or synthetic plant sterols, lunasin peptide/s and extract of a plant from the genus Moringa or any combinations thereof and at least one polyethoxylated castor oil or any derivative thereof.

In some specific embodiments, the composition of the invention may comprise as an active ingredient at least one natural or synthetic plant sterols, specifically, CardioAid and at least one polyethoxylated castor oil or any derivative thereof.

In yet some further embodiments, the composition of the invention may comprise at least one lunasin peptide/s and at least one polyethoxylated castor oil or any derivative thereof.

In still some further embodiments, the composition of the invention may comprise at least one extract of a plant from the genus Moringa or any combinations thereof and at least one polyethoxylated castor oil or any derivative thereof.

In yet other specific embodiments the optional component consisting of adjuvants, namely at least one adjuvant selected from group of polyethylene glycol, polyethoxylated castor oil; beta cyclo dextrin or a derivative thereof; may be a polyethoxylated castor oil that is Cremophore EL (C:E).

Castor oil as meant herein relates to a natural vegetable oil obtained from seeds of the castor oil plant (Ricinus communis). The FDA categorized castor oil as GRASE (Generally Recognized As Safe and Effective). Castor oil, or synthetic castor oil derivatives such as polyethoxylated castor oil, are available over-the-counter for use as laxatives, and were also approved for human use as vehicles for oral and intravenous administration of water-insoluble therapeutic compounds. In naturopathy, castor oil has been promoted as a treatment for a variety of human health conditions.

The term ‘Ethoxylated Castor Oil’ (also Polyoxyl Castor Oil, Polyoxyl Castor Oil, Polyethylene Glycol Castor Oil, Castor Oil Ethoxylates and Polyethoxylated Castor Oil) refers to a nonionic surfactant having many industrial applications. Polyoxyethylene castor oil derivatives are complex mixtures of various hydrophobic and hydrophilic components. In the polyethoxylated castor oil, the hydrophobic constituents comprise about 80% of the total mixture, the main component being glycerol polyethylene glycol ricinoleate. Other hydrophobic constituents include fatty acid esters of polyethylene glycol along with some unchanged castor oil. The hydrophilic part consists of polyethylene glycols and glycerol ethoxylates.

Further, ethoxylated castor oil is also referred to as a mixture of triricinoleate esters of ethoxylated glycerol with small amounts of polyethyleneglycol (macrogol) ricinoleate and the corresponding free glycols. Polyoxyethylene castor oil derivatives are nonionic surfactants used in oral, topical and parenteral pharmaceutical formulations.

As noted above, in certain embodiments, the derivative of polyethoxylated castor oil of the compositions of the invention is Cremophor EL or more recently Kolliphor EL (registered trademark of BASF Corp) and also polyoxyethylenglyceroltriricinoleat 35 (DAC), polyoxyl 35 castor oil (USP/NF). Cremophor EL (herein also C:E) is obtained by reacting ethylene oxide with castor oil (molar ratio 35:1). The main component of C:E is glycerol-polyethylene glycol ricinoleate, which, together with fatty acid esters of polyethylene glycol, represents the hydrophobic part of the product. The smaller, hydrophilic part consists of polyethylene glycols and ethoxylated glycerol. Due to this particular composition, C:E is capable to stabilize emulsions of nonpolar materials in aqueous solutions, thus making it a universal nonionic emulsifying agent for the pharmaceutical, cosmetic and food industries. Some anti-neoplastic agents (e.g. Taxol, Taxotere) were formulated in C:E and ethanol to enhance drug solubility and therapeutic effect. When describing the present invention, the terms emulsifying agents, excipient and surfactant are interchangeable.

Specifically, Cremophor EL (CAS Registry number 63393-92-0) (Synonyms Macrogolglycerol ricinoleate, PEG-35 castor oil, Polyoxyl 35 hydrogenated castor oil, Polyoxyl-35 castor oil) denotes a derivative of castor oil or an ester with ethoxylated glycerol of Molecular Formula C5H1204; Molecular Weight:136.14638 [g/mol]; Formal Charge:0; Boiling Point 290° C. at 760 mmHg; Flash Point 160° C.

In yet some specific embodiments, derivative of polyethoxylated castor oil applicable in the present invention may be at least one of Cremophor® EL (Polyoxyl 35 Castor Oil, NF) and Cremophor® RH40 (Polyoxyl 40 Hydrogenated Castor Oil, NF), also referred to as Kolliphor® RH 40.

Further, the term C:E designates preparation of Cremophor EL in ethanol (1:1 v/v) and it represents 30% v/v when emulsified in PBS. According to some specific embodiments, the Cremophore EL may be dissolved in or combined with EtOH. More specifically, the C and the E (EtOH) ratio may range between about 1:0 to 1:999999, more specifically, 1:1 to 1:99999, 1:1 to 1:9999, 1:1 to 1:999, 1:1 to 1:99, 1:1 to 1:9. Nevertheless, it should be appreciated that the Cremophor of the invention may be prepared or dissolved in any other solvent.

In certain other embodiments, the combined composition of the invention may comprise at least one of natural or synthetic plant sterols, lunasin peptide/s and extract of a plant from the genus Moringa or any combinations thereof and an adjuvant such as any one of polyethylene glycol or beta cyclo dextrin or any derivative thereof. The term ‘adjuvant’ as used herein refers to a pharmacological agent that modifies and enhances the effect of other active agents. It should be noted that the specific adjuvants indicated herein were now surprisingly shown by the invention as exerting a therapeutic effect/s as active main ingredients and not only as additional enhancing or inherent agents.

In some alternative specific embodiments, the combined compositions of the invention may comprise an adjuvant such as polyethylene glycol or any derivatives thereof. Polyethylene glycol (PEG) is a polyether compound PEG is also known as polyethylene oxide (PEO) or polyoxyethylene (POE), depending on its molecular weight. PEG, PEO, or POE refers to an oligomer or polymer of ethylene oxide. The three names are chemically synonymous, PEG refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. PEG is used as an excipient in many pharmaceutical products.

PEG is soluble in water, methanol, ethanol, acetonitrile, benzene, and dichloromethane, and is insoluble in diethyl ether and hexane. It is coupled to hydrophobic molecules to produce non-ionic surfactants. When attached to various protein medications, polyethylene glycol allows a slowed clearance of the carried protein from the blood. This makes for a longer-acting medicinal effect and reduces toxicity, and allows longer dosing intervals.

PEG is used as an excipient in many pharmaceutical products. Lower-molecular-weight variants are used as solvents in oral liquids and soft capsules, whereas solid variants are used as ointment bases, tablet binders, film coatings, and lubricants.

In more specific embodiments, The term ‘Polyethylene Glycol’ (CAS Registry number 25322-68-3; CA Index Name: Poly(oxy-1,2-ethanediyl), α-hydro-ω-hydroxy-) denotes an addition polymer of ethylene oxide and water, represented by the formula H(OCH2CH2)nOH, denoted herein as Formula I:

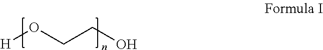

in which n represents the average number of oxyethylene groups. In some embodiments, the average molecular weight is not less than 95.0% and not more than 105.0% of the labeled nominal value if the labeled nominal value is below 1000; it is not less than 90.0% and not more than 110.0% of the labeled nominal value if the labeled nominal value is between 1000 and 7000; it is not less than 87.5% and not more than 112.5% of the labeled nominal value if the labeled nominal value is above 7000. It may contain a suitable antioxidant.

The term PEG designates preparation of PEG in ethanol (1:1 v/v) and it represents 30% v/v when emulsified in PBS. According to some specific embodiments, the PEG may be dissolved in or combined with EtOH. More specifically, the PEG and the E (EtOH) ratio may range between about 1:0 to 1:999999, more specifically, 1:1 to 1:99999, 1:1 to 1:9999, 1:1 to 1:999, 1:1 to 1:99, 1:1 to 1:9.The PEG of the invention may be prepared or dissolved in any other solvent.

Still further alternative embodiments of the invention encompass use of at least one of natural or synthetic plant sterols, lunasin peptide/s and extract of a plant from the genus Moringa or any combinations thereof and an adjuvant such as beta cyclo dextrin (BCD). Cyclodextrins (sometimes called cycloamyloses) are a family of compounds made up of sugar molecules bound together in a ring (cyclic oligosaccharides). Cyclodextrins are produced from starch by means of enzymatic conversion. They are used in food, pharmaceutical, drug delivery, and chemical industries, as well as agriculture and environmental engineering. Cyclodextrins are composed of 5 or more α-D-glucopyranoside units linked 1→4, as in amylose (a fragment of starch). The 5-membered macrocycle is not natural. Recently, the largest well-characterized cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape: α (alpha)-cyclodextrin: 6-membered sugar ring molecule; β (beta)-cyclodextrin: 7-membered sugar ring molecule; γ (gamma)-cyclodextrin: 8-membered sugar ring molecule. α- and γ-cyclodextrin are being used in the food industry. As α-cyclodextrin is a soluble dietary fiber, it can be found as Alpha Cyclodextrin (soluble fiber) on the list of ingredients of commercial products.

Because cyclodextrins are hydrophobic inside and hydrophilic outside, they can form complexes with hydrophobic compounds. Thus they can enhance the solubility and bioavailability of such compounds. This is of high interest for pharmaceutical as well as dietary supplement applications in which hydrophobic compounds shall be delivered. Alpha-, beta-, and gamma-cyclodextrin are all generally recognized as safe by the FDA. In the food industry, cyclodextrins are employed for the preparation of cholesterol free products. More specifically, the term ‘β-Cyclodextrin’ (CAS Registry number 7585-39-9; Synonyms Cycloheptaamylose, Cyclomaltoheptaose, β-cycloamylose, cycloheptaglucan, cycloheptaglucosan, Betadex) denotes a cyclodextrin composed of seven α-(1→4) linked D-glucopyranose units C₄₂H₇₀O₃₅; Molecular Weight 1134.98[g/mol]

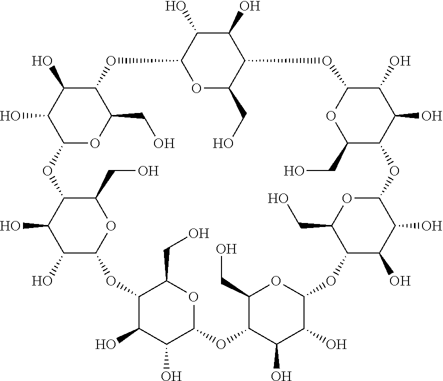 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In more specific embodiments, the invention provides combined compositions comprising as active ingredients SE and Methyl-β-cyclodextrin.

Both β-cyclodextrin and methyl-β-cyclodextrin (M(βCD) remove cholesterol from cultured cells. The methylated form MβCD was found to be more efficient than β-cyclodextrin. The water-soluble MβCD is known to form soluble inclusion complexes with cholesterol, thereby enhancing its solubility in aqueous solution. MβCD is employed for the preparation of cholesterol-free products: the bulky and hydrophobic cholesterol molecule is easily lodged inside cyclodextrin rings that are then removed. MβCD is also employed in research to disrupt lipid rafts by removing cholesterol from membranes.

The term BCD designates preparation of BCD in ethanol (1:1 v/v) and it represents 30% v/v when emulsified in PBS. According to some specific embodiments, the BCD may be dissolved in or combined with EtOH. More specifically, the BCD and the E (EtOH) ratio may range between about 1:0 to 1:999999, more specifically, 1:1 to 1:99999, 1:1 to 1:9999, 1:1 to 1:999, 1:1 to 1:99, 1:1 to 1:9. Nevertheless, it should be appreciated that the BCD of the invention may be prepared or dissolved in any other solvent.

It should be noted that the compositions of the present invention may comprise any combination, including partial combinations, of the above mentioned components.

As noted above, the combined compositions of the invention comprise at least two active agents, specifically, at least one of natural or synthetic plant sterols, lunasin peptide/s and extract of a plant from the genus Moringa or any combinations thereof and optionally, at least one of SE, beta-glycolipid/s and different adjuvants. It should be appreciated that any quantitative ratio of the combined compounds may be used. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, specifically, where additional therapeutic agents are added, the quantitative ratio used may be for example, 1:1:1, 1:2:3, 1:10:100, 1:10:100:1000 etc.

It is further contemplated that in some embodiments, compositions of the invention may be formulated as a food additive, food supplement or medical food. In alternative embodiments, compositions of the invention may be further added or combined with botanical drugs, drugs or any type of pharmaceutical products.

It is conceived that further embodiments of the present invention pertains to compositions in a formulation adapted for add-on to a solid, semi-solid or liquid foods, and to various types of beverages.

More specifically, in certain embodiments, the combined composition of the invention may be an add-on to a food supplement, or alternatively, may be used as a food supplement. A food supplement, the term coined by the European Commission for Food and Feed Safety, or a dietary supplement, an analogous term adopted by the US Food and Drug Administration (FDA), relates to any kind of substances, natural or synthetic, with a nutritional or physiological effect whose purpose is to supplement the normal diet. In this sense, this term also encompasses food additives and dietary ingredients. Further, under the Dietary Supplement Health and Education Act of 1994 (DSHEA), a statute of US Federal legislation, the term dietary supplement is defined as a product (other than tobacco) intended to supplement the diet that bears or contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any of the aforementioned ingredients.

Under food or dietary supplements is meant those marketed in a form of pills, capsules, powders, drinks, and energy bars and other dose forms. Unlike drugs, however, they are mainly unregulated, i.e. marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet.

In yet some further embodiments, the combined composition of the invention may be an add-on to medical foods. Further in this connection should be mentioned medical foods, which are foods that are specially formulated and intended for the dietary management of a disease that has distinctive nutritional needs that cannot be met by normal diet alone. The term medical food, as defined in the FDA's 1988 Orphan Drug Act Amendments is a food which is formulated to be consumed or administered enterally under the supervision of a physician and which is intended for the specific dietary management of a disease or condition for which distinctive nutritional requirements, based on recognized scientific principles, are established by medical evaluation.

Hence, medical foods are subject to the general food and safety labeling requirements of the Federal Food, Drug, and Cosmetic Act. Medical foods are usually classified as nutritionally complete or incomplete formulas, formulas for metabolic disorders and oral rehydration products. Notable examples of the above include gamma-linolenic acid (GLA) and/or a short chain omega-6 fatty acid sourced from the seeds of the borage plant for management of allergic conditions; slowly digested carbohydrates for maintenance of optimal blood sugar levels especially in patients with diabetes; and glutamine for nourishment of the gastrointestinal (GI tract) in metabolically stressed patients.

Also pertinent to the present context are botanical drugs. In specific embodiments, compositions of the invention may be an add-on to a botanical drug. As used herein botanical drug are products that are intended for use in the diagnosis, cure, mitigation, treatment or prevention of disease in humans. A botanical drug product consists of vegetable materials, which may include plant materials, algae, macroscopic fungi, or combinations thereof. A botanical drug product may be available as (but not limited to) a solution (e.g., tea), powder, tablet, capsule, elixir, topical, or injection. Botanical drug products often have unique features, for example, complex mixtures, lack of a distinct active ingredient, and substantial prior human use. Fermentation products and highly purified or chemically modified botanical substances are not considered botanical drug products. According to the FDA Guidance for Industry, a botanical product may be a food (including a dietary supplement), a drug (including a biological drug), a medical device (e.g., gutta-percha), or a cosmetic. Further, botanical drugs may include botanical ingredients in combination with either a synthetic or highly purified drug or a biotechnology derived or other naturally derived drug. In the same way, botanical drugs may also contain animals or animal parts (e.g., insects, annelids, shark cartilage) and/or minerals or a combination thereof.

Specifically pertinent to the present context are foods or food supplements based on soybean (US) or soya bean (UK) or any soy-derived extract.

Of particular relevance are applications using compositions of the present invention in the form of add-on to foods or beverages comprising high content of sugar and/or alcohol, which are associated with altered blood sugar levels, altered insulin resistance and/or hepatic function.

In other words, it is meant that in specific embodiments various compositions of the invention may be applicable for controlling blood sugar levels in a subject, wherein said control is inhibiting increase or decrease in blood sugar levels, improving glucose tolerance or altering insulin resistance state.

In other specific embodiments, compositions of the invention may be applicable for altering insulin resistance and/or hepatic function.

Of particular interest are certain embodiments in which compositions of the present invention are used as add-on to foods and/or beverages comprising an increased content of sugar and/or alcohol or are associated with increase in blood sugar and/or alcohol level.

In a broader sense, compositions of the invention may be adapted for add-on to food and/or beverage that comprise an increased content of sugar and/or alcohol or to a food or beverage that may be associated with increase in blood sugar or alcohol level via alteration of the insulin resistance state or the capability to alter alcohol metabolism by the body.

In this context, a sugar sweetened beverage (SSB) is any beverage with added sugar, including for example fruit or fruit-flavored drinks, flavored water or sodas, energy drinks (also referred to as soft drinks), as well as chocolate milk, coffees, teas and nonalcoholic wines and beers. For the purpose of describing the invention, the terms added sugar, sugar sweetened and high sugar content are interchangeable. Risks of weight gain, obesity and diabetes which have been linked to consumption of sweetened beverages will be discussed further below.

An alcoholic beverage is a drink typically containing 0.1-95% alcohol, most commonly ethanol but occasionally also other alcohols. Alcoholic beverages include beers, wines, and spirits (distilled beverages). For the purpose of the present invention, the term an alcoholic beverage encompasses any kind of alcohol containing beverage produced by process of fermentation or distillation or both, or any type of food or drink that directly or indirectly affect the metabolism of alcohol. Consequences of alcohol consumption, such as alcohol intoxication, hangover and liver damage, well as the link between alcohol consumption and blood sugar levels, are discussed further below.

As meant herein, the terms blood sugar level or blood glucose level imply molar concentration of glucose in the blood or serum of an organism (human or animal) Glucose being, with some exceptions, the primary source of energy for all body's cells, is transported from the intestines or liver to body cells via the bloodstream and is made available for cell absorption via the hormone insulin produced primarily in the pancreas. The body's homeostatic mechanism keeps blood glucose levels within a narrow range by means of several interacting systems, of which hormone regulation is the most important. There are two types of mutually antagonistic metabolic hormones affecting blood glucose levels: (1) catabolic hormones (such as glucagon, cortisol and catecholamines) which increase blood glucose; and (2) an anabolic hormone (insulin) which decreases blood glucose.

Glucose levels are usually lowest in the morning, before the first meal of the day (termed the fasting level) and rise after meals for an hour or two by a few millimolar. Blood sugar levels outside the normal range may be an indicator of certain medical conditions. A persistently high level is referred to as hyperglycemia; low levels are referred to as hypoglycemia. Diabetes mellitus is characterized by persistent hyperglycemia from any of several causes, and is the most prominent disease related to failure of blood sugar regulation. Intake of alcohol causes an initial surge in blood sugar, and later tends to cause levels to fall. Certain drugs can also increase or decrease glucose levels.

Under the term normal or recommended blood glucose levels is meant, in humans, the mean normal levels (tested while fasting) are between 70 to 100 mg/dL (3.9 to 5.5 mmol/L) and are restored within this range, if the body's homeostatic mechanism is operating normally. According to the American Diabetes Association, blood sugar levels for those without diabetes and who are not fasting should be below 125 mg/dL. The blood glucose target range for diabetics should be 90-130 mg/dL before meals and less than 180 mg/dL after meals.

According to other estimates, the normal blood glucose level in humans in fasting is approximately 4 mmol/L (4 mM or 72 mg/dL); shortly after a meal the blood glucose level may rise temporarily up to 7.8 mM (140 mg/dL); when operating normally the body restores blood sugar levels to a range of 4.4 to 6.1 mM (82 to 110 mg/dL). For people with type 1 or type 2 diabetes blood sugar level targets are: before meals—4 to 7 mM for; after meals—under 9 mM for people with type 1 and 8.5 mM for people with type 2; children with type 1 diabetes have a greater upper limit for their blood sugar levels by 1 mM.

In this connection, it should be also understood under blood glucose levels is meant arterial, venous and capillary blood glucose levels, which may be comparable or distinct, when fasting or after meals.

Further, the present invention may be applicable in conjunction with measurements or monitoring of blood glucose levels using any available technology, including direct-to-customer glucose blood testing, such as disposable test-strips or electronically-based devices. This is particularly applicable for subjects with diabetes or insulin resistance.

Another application of blood glucose monitoring is a glucose tolerance test, a medical test in which glucose is given and blood samples taken afterward to determine how quickly it is cleared from the blood. This test is usually used to test for diabetes, insulin resistance, and sometimes reactive hypoglycemia and acromegaly, or rarer disorders of carbohydrate metabolism. In the most commonly performed version of the test, an oral glucose tolerance test (OGTT), a standard dose of glucose is ingested by mouth and blood levels are checked two hours later. Many variations of the GTT have been devised over the years for various purposes, with different standard doses of glucose, different routes of administration, different intervals and durations of sampling, and various substances measured in addition to blood glucose. Usually the OGTT is performed in the morning as glucose tolerance can exhibit a diurnal rhythm with a significant decrease in the afternoon. The patient is instructed to fast (water is allowed) for 8-12 hours prior to the tests. The oral glucose challenge test (OGCT) is a short version of the OGTT, used to check pregnant women for signs of Gestational Diabetes. It can be done at any time of day, not on an empty stomach. The test involves 50 g of glucose, with a reading after one hour.

Since the 1970s, the World Health Organization and other organizations interested in diabetes agreed on a standard dose and duration. According to standard OGTT protocol:
A zero time (baseline) blood sample is drawn.
The patient is then given a measured dose of glucose solution to drink within 5 min
Blood is drawn at intervals for measurement of glucose, and sometimes insulin levels.

The intervals and number of samples vary according to the purpose of the test. For simple diabetes screening, the most important sample is the 2 hour sample and the 0 and 2 hour samples may be the only ones collected. A laboratory may continue to collect blood for up to 6 hours depending on the protocol requested by the physician. Fasting plasma glucose (measured before the OGTT begins) should be below 6.1 mmol/L (110 mg/dL). Fasting levels between 6.1 and 7.0 mmol/L (110 and 125 mg/dL) are borderline (“impaired fasting glycaemia”), and fasting levels repeatedly at or above 7.0 mmol/L (126 mg/dL) are diagnostic of diabetes. A 2 hour OGTT glucose level below 7.8 mmol/L (140 mg/dL) is normal, whereas higher glucose levels indicate hyperglycemia. Blood plasma glucose between 7.8 mmol/L (140 mg/dL) and 11.1 mmol/L (200 mg/dL) indicate impaired glucose tolerance and levels above 11.1 mmol/L (200 mg/dL) at 2 hours confirms a diagnosis of diabetes. For the 75 g OGTT: fasting should be below 5.1 mmol/L; 1 hour should be below 10.0 mmol/L; 2 hour should be below 8.5 mmol/L.

Glucose tolerance test is particularly relevant for the diagnosis of insulin resistance state. Insulin resistance describes the body's lack of sensitivity to the hormone insulin, meaning body cells such as the muscle, fat and liver cells are not adequately stimulated to take up glucose from the blood, even when insulin levels are high. This under-utilization of blood glucose results in hyperglycemia or a raised blood sugar level. Tests for diagnosing insulin resistance include:
Fasting blood sugar and postprandial blood sugar—Blood sugar is almost always raised in people with insulin resistance.
Fasting insulin assessment—In a healthy person who has fasted for 6 to 8 hours (usually overnight), the insulin level is approximately 60 pmol/L. A level higher than this is considered indicative of insulin resistance.
Glucose tolerance testing (GTT)—For a glucose tolerance test, a person fasts for 8 to 12 hours (usually overnight) and is then given a 75 gram oral dose of glucose. After two hours, the blood levels of glucose are measured.
In a healthy person, the blood sugar level after two hours is usually less than 7.8 mmol/L (140 mg/dl). A blood sugar level between 7.8 and 11.0 mmol/dl (140 to 197 mg/dl), however, indicates impaired glucose tolerance. If the level is over 11.1 mmol/dl (200 mg/dl), diabetes mellitus is diagnosed.
Modified insulin suppression test—For this test, patients are given 25 mcg of octreotide (an inhibitor of insulin and glucagon) over 3 to 5 minutes and are then infused with somatostatin (0.27 μgm/m2/min) to suppress the release of insulin and glucose into the blood.

As previously mentioned, temporary fluctuations of blood glucose levels may develop under various conditions, among which consumption of sugar sweetened or alcoholic beverages represent a significant contributing factor. In addition, alteration of blood sugar levels can occur following use of medications or in other states altering the level of insulin resistance.

Particularly relevant to the present context are alcohol containing beverages. Alcohol interferes with all three sources of glucose and hormones needed to maintain healthy blood glucose levels. The greatest impact is seen in those who drink heavily and on frequent basis. In heavy drinkers, glycogen stores are depleted within few hours, if their diet does not provide a sufficient amount of carbohydrates. Over time, excessive alcohol consumption can decrease insulin's effectiveness, resulting in high blood sugar levels; according to certain estimates 45% to 70% of people with alcoholic liver disease had either glucose intolerance or diabetes.

Alcohol can also negatively impact blood sugar levels each time that it is consumed, regardless of the frequency of consumption. Research has shown that acute consumption increases insulin secretion causing low blood sugar (hypoglycemia) and leading to impairment of hormonal responses that would normally rectify the low blood sugar. Drinking as little as 2 ounces of alcohol on an empty stomach can lead to very low blood sugar levels. This makes alcohol an even bigger problem for people with diabetes. Along with the impact on blood sugar, studies have also shown that alcohol can impact the effectiveness of the hypoglycemic medications, so extreme caution needs to be taken when consuming alcohol by anyone with diabetes.

There is also an increased risk of problems when combining exercise and alcohol. While blood sugars naturally drop during exercise and a body is working on replacing its glycogen stores, consuming alcohol during this time will halt this process and can cause blood sugar levels to stay at an unhealthy level.

The present meaning of alcohol consumption encompasses the entire range of associated physiological, psychological, social conditions, i.e. social drinking, session drinking, binge drinking alcohol abuse, alcohol intoxication and alcoholism. Meaning of these terms in the present context is detailed below.

Further, it is conceived that compositions of the present invention are used for prevention or alleviation of symptoms related to a condition associated with increased or decreased blood sugar levels, wherein said condition is any one of pre-diabetes, diabetes, obesity, hepatic disorder, pancreatic dysfunction, weight gain, alcohol intoxication, alcohol withdrawal and vertigo, any condition associated with alteration of pancreatic or liver function or tissue or organ damage.

It should be therefore understood that compositions of the present invention are particularly applicable to the prevention or alleviation of symptoms related to sub-clinical conditions associated with altered insulin resistance state and/or hepatic function, such as pre-diabetes, diabetes, obesity, hepatic disorder, pancreatic dysfunction, weight gain, alcohol intoxication, alcohol withdrawal and vertigo, any condition associated with alteration of pancreatic or liver function or tissue or organ damage, and drug-induced hepatic dysfunction.

It should be therefore appreciated that the active ingredients used by the compositions and methods of the invention, specifically, at least one of natural or synthetic sterol or a derivative or a mixture thereof, lunasin peptide or a derivative thereof; and at least one extract of a plant from the genus Moringa, are presented in an amount effective for treatment, prevention or alleviation of any of the disorders indicated herein as well as of any of any condition associated therewith.

In this context, “weight gain” is meant an increase in body weight, particularly by way of increased body fat deposits (adipose tissue), than is optimally healthy. A person generally gains fat-related weight by increasing food consumption or by becoming physically inactive, or by both. When energy intake exceeds energy expenditure, the body stores the excess energy in a dense high-energy form as fat. One pound of fat stores 3500 calories of energy, so over time, excessive energy intake and/or lack of exercise can contribute to fat gain and obesity. Having excess fat is a common condition, as much as 64% of the US adult population is considered either overweight or obese, and this percentage has increased over the last four decades. Weight gain has a latency period. The effect that eating has on weight gain can vary greatly depending on the following factors: energy (calorie) density of foods, exercise regimen, amount of water intake, amount of salt contained in the food, time of day eaten, age of individual, individual's country of origin, individual's overall stress level and amount of water retention in ankles/feet. Typical latency periods vary from three days to two weeks after ingestion. Weight gain is also a common side-effect of certain psychiatric medications. Weight gain is seen in certain professional sports.

In this connection, the present invention is relevant to prevention of weight gain in all its measurements and forms. One of the ways to assess abnormal weight is by the measurement of Body Mass Index (BMI), or Quetelet index, which is a measure of relative weight based on an individual's mass and height. The WHO regards a BMI of less than 18.5 as underweight indicative of malnutrition, an eating disorder or other health problems, while a BMI greater than 25 is considered overweight and above 30 is considered obese.

Increase in body fat percentage or an excess of adipose tissue on a human can lead to serious health side-effects. A large number of medical conditions have been associated with obesity. Health consequences are categorized as being the result of either increased fat mass (osteoarthritis, obstructive sleep apnea, social stigma) or increased number of fat cells (diabetes, some forms of cancer, cardiovascular disease, non-alcoholic fatty liver disease). There are alterations in the body's response to insulin (insulin resistance), a proinflammatory state and an increased tendency to thrombosis (prothrombotic state). The ever-present social stigma concerning weight gain can have lasting and harmful effects, especially among young women.

In certain embodiments, compositions of the present invention are particularly applicable to prevention and reduction of symptoms of alcohol intoxication, alcohol withdrawal and vertigo. Symptoms of alcohol intoxication include reduced activity in the central nervous system (CNS), loose muscle tone, loss of fine motor coordination, a staggering “drunken” gait, eyes appear “glossy,” pupils may be slow to respond to stimulus, pupils may become constricted, decreased heart rate, lower blood pressure and respiration rate, decreased reflex responses, slower reaction times, skin may be cool to the touch, profuse sweating, loss of fine motor coordination, or odor of alcohol on the breath. Diagnostic criteria for alcohol intoxication are detailed in the Diagnostic and Statistical Manual of Mental Disorders, Fourth Edition (DSM-IV).

The term alcohol intoxication as used herein refers to a situation where the quantity of alcohol a person consumes exceeds the individual's tolerance for alcohol and thus produces, either during or shortly after drinking, clinically important psychological, behavioral or physical abnormalities, such as inappropriate aggression, and impaired judgment and social functioning. One or more of the following signs or symptoms of alcohol intoxication occur shortly after drinking: (1) slurred speech; (2) impaired motor coordination; (3) unsteady gait; (4) nystagmus (involuntary, irregular eye movement characterized by smooth pursuit of an object in one direction and saccadic movement in the other direction); (5) inattention and/or impaired memory; and (6) stupor or coma.

Sobriety, intoxication, alcohol abuse, alcohol-related aggression or alcoholism may be measured according to one or more recognized tests, such as psychomotor tests, serum alcohol level tests, for example accepted inhalation tests, or according to DSM-IV, Alcohol Abstinence Self-Efficacy Scale, Barratt Impulsiveness Scale—11, State-Trait Anger Expression Inventory—2, Conflict Resolution, Impulsivity and Aggression Questionnaire, Social Problem-Solving Inventory—Revised, Alcohol-Related Aggression Questionnaire, or The Alcohol Use Disorders Identification Test. Levels of alcohol in the body may be measured in urine, blood, breath or saliva.

There is a wide range of variability in blood alcohol levels that different individuals can tolerate without becoming intoxicated. The range may be as great as from 0.3 to 1.5 mg/ml, although most states in the U.S. set the sobriety level for legally driving at 0.8 mg/ml. Some users may develop significant behavioral changes or become intoxicated at a much lower Blood Alcohol Concentration (BAC) than the legal limit. This condition is known as “Alcohol Idiosyncratic Intoxication” or “Pathological Intoxication”. In general:
0.02-0.03 BAC no loss of coordination, slight euphoria and loss of shyness.
0.04-0.06 BAC feeling of well-being, relaxation, lower inhibitions, sensation of warmth, euphoria, some minor impairment of reasoning and memory.
0.07-0.09 BAC slight impairment of balance, speech, vision, reaction time; reduction of judgment and self-control and caution, reason and memory.
0.10-0.125 BAC significant impairment of motor coordination and loss of good judgment; slurred speech; balance, vision, reaction time and hearing are impaired; euphoria. It is illegal to operate a motor vehicle at this level.
0.13-0.15 BAC gross motor impairment and lack of physical control; blurred vision and major loss of balance; euphoria is reduced and dysphoria (anxiety, restlessness) is beginning to appear.
0.16-0.20 BAC dysphoria predominates, nausea may appear.
0.25 BAC the drinker needs assistance in walking; total mental confusion.
0.30 BAC loss of consciousness.
0.40 BAC and up onset of coma, possible death due to respiratory arrest.

The term social drinking refers to the consumption of alcohol in a safe, legal and socially acceptable manner usually without the intent of reaching the point of becoming intoxicated (i.e., to achieve alcohol intoxication). Although the amount of blood alcohol which leads to intoxication varies widely between individuals, three or fewer measured drinks (or a blood alcohol level of up to 0.05%) is generally considered to be within the social drinking range.

The term session drinking refers to drinking in large quantities over a single period of time, without the intention of getting heavily intoxicated. The focus is on the social aspects of the occasion.

The term binge drinking refers to drinking alcohol solely for the purpose of intoxication, although it is quite common for binge drinking to apply to a social situation, creating some overlap in social and binge drinking.

The term alcoholism refers to a primary chronic disease known as alcohol dependence syndrome, the most severe stage of a group of drinking problems. Alcoholism is considered a progressive disease, meaning that the symptoms and effects of drinking alcohol become increasingly more severe over time.

The term alcohol abuse refers to repeated drinking despite alcohol-related physical, social, psychological, or occupational problems (according to DSM-IV). When alcohol abuse reaches the alcohol dependence stage, a person may also experience tolerance, withdrawal, and an uncontrolled drive to drink.

In the context of the present invention, after-effects of alcohol consumption, specifically alcohol hangover, alcohol withdrawal or detoxification are also included, as well as any effect of the alcohol on target organs such as the liver, heart, kidney, brain, muscles, gastrointestinal tract, and any other tissue or organ that can be affected by alcohol or by compounds or states in which the metabolism of alcohol is disturbed.

Alcohol hangover refers to physical and mental symptoms that occur within several hours after alcohol consumption, when a person's BAC is falling, and may continue for up to 24 hours thereafter. Alcohol directly promotes hangover symptoms through its effects on urine production, the gastrointestinal tract, blood sugar concentrations (i.e. hypoglycemia), sleep patterns, and biological rhythms. In addition, effects related to alcohol absence after a drinking bout (i.e., withdrawal), alcohol metabolism, and other factors (e.g., biologically active, non-alcohol compounds in beverages, use of other drugs, certain personality traits and a family history of alcoholism) also may contribute to the hangover condition. The particular set of symptoms experienced and their intensity may vary from person to person and from occasion to occasion. In addition, hangover characteristics may depend on the type of alcoholic beverage consumed and the amount a person drinks.

Physical symptoms of a hangover include fatigue, headache, increased sensitivity to light and sound, redness of the eyes, muscle aches, and thirst. Signs of increased sympathetic nervous system activity can accompany a hangover, including increased systolic blood pressure, rapid heartbeat (i.e., tachycardia), tremor, and sweating. Mental symptoms include dizziness, sense of the room spinning (i.e., vertigo), and possible cognitive and mood disturbances, especially depression, anxiety, and irritability.

Alcohol-induced hypoglycemia generally occurs after binge drinking over several days in alcoholics who have not been eating. In such a situation, prolonged alcohol consumption, coupled with poor nutritional intake, not only decreases glucose production but also exhausts the reserves of glucose stored in the liver in the form of glycogen, thereby leading to hypoglycemia. Because glucose is the primary energy source of the brain, hypoglycemia can contribute to hangover symptoms such as fatigue, weakness, and mood disturbances. Diabetics are particularly sensitive to the alcohol-induced alterations in blood glucose.

Several lines of evidence suggest that a hangover and mild alcohol withdrawal (AW) share a common biological mechanism. First, the signs and symptoms of hangover and mild AW overlap considerably. Second, it has been known that alcohol re-administration alleviates the unpleasantness of both AW syndrome and hangovers.

In further embodiments, compositions of the invention may be applicable for AW and AW syndrome. The AW or AW syndrome or alcohol detoxification, the terms used herein interchangeably, refers to the state following the cessation of excessive drinking, which results from compensatory changes in the CNS that take place in response to chronically administered depressant substances (in this case, alcohol, or more specifically, ethanol). These changes include alterations in the GABA and glutamate receptors, the two main neurotransmitters responsible for inhibitory and excitatory effects. Following chronic alcohol exposure, in an effort to counterbalance the alcohol's sedative effects, the body decreases the number or sensitivity of GABA receptors and increases the number or sensitivity of glutamate receptors. When alcohol is removed from the body, the CNS and a portion of the sympathetic nervous system that coordinates response to stress remain in an unbalanced “overdrive” state. Sympathetic nervous system hyperactivity accounts for the tremors, sweating, and tachycardia observed in both hangover and AW syndrome.

In still further embodiments, compositions of the present invention are applicable to prevention of vertigo, i.e. a subtype of dizziness in which a patient inappropriately experiences the perception of motion (usually a spinning motion) due to dysfunction of the vestibular system. It is often associated with nausea and vomiting as well as a balance disorder, causing difficulties with standing or walking Dizziness and vertigo are common and affect approximately 20%-30% of the general population, they can occur in people of all ages, in women more than in men.

Apart from physiological causes of vertigo, such as infections of the inner ear, concussion, migraine, epilepsy and others, excessive drinking of alcohol can also cause symptoms of vertigo.

Examples of clinical applications using pharmaceutical compositions of the invention may include disorders such as diabetes, obesity, various hepatic disorders, disorders involving pancreatic dysfunction, insulin resistance and metabolic syndrome, and further an inflammation of pancreas, liver, muscle or the adipose tissue, other inflammatory disorders and also arrange of malignancies. Other relevant disorders will be detailed further below.

Thus, it is contemplated that a composition of the invention is used in a method for treating, preventing, ameliorating, reducing or delaying the onset of an immune-related disorder, said composition comprising a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the composition of the invention may optionally further comprise at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

In specific embodiments, an immune-related disorder that is treated by compositions and method of the invention may be any one of an inflammatory disorder, an autoimmune disorder, an infectious disease and a proliferative disorder.

In specific embodiments of such applications, the compositions of the invention may further comprises at least one additional therapeutic agent.

In more specific embodiments, such compositions may comprise at least one additional therapeutic agent as insulin, N-acetyl cysteine (NAC), thiamine (vitamin B1), a benzodiazepine or any combination thereof and a tissue derived preparation or compound.

It is further conceived that for the purpose of specific embodiments and methods, the combined composition of the invention may be an add-on to any type of drugs or therapeutic compounds administered orally, intravenously, intradermaly, by inhalation or intrarectaly. Examples of such combined compositions include, but are not limited to a tissue derived antigens, tumor associated antigens, or viral and or bacterial and or fungal and or parasitic or bacterial derived antigens, or any type of organism derived antigens. It should be further noted that the compositions of the invention may be add-on to any type of healthy of diseased tissue derived antigens, or any type of drug or therapeutic compound, or any type of organism derived antigens, or hormones, or cytokines, or antibody, or any type of natural or non-natural compound that may have therapeutic properties. More specifically, such add-on preparation may be used for promoting the effect of said therapeutic compound, for exerting an adjuvant effect, or for improving the therapeutic effect of said drug, compound, or antigen.

Of particular relevance to this context, compositions of the invention as add-on products to hormones, including but not limited to insulin, whether natural or synthetic.

In other specific embodiments, compositions of the invention may be add-on products to at least one gut hormone. Yet in alternative embodiments, the combined composition of the invention may be used as add-on products for concomitant administration of at least one gut hormone. In more particular but non-limiting embodiments, gut hormone include Ghrelin, Cholecystokinin, Cholecystokinin, Peptide YY, Pancreatic polypeptide, Amylin, Glucose-dependent insulinotropic polypeptide, Glucagon-like peptide-1, Glucagon-like peptide-2 and Oxyntomodulin. In more specific embodiments the combined composition of the invention may comprise Ghrelin. As used herein Ghrelin is a peptide hormone released from the stomach and liver and is often referred to as the “hunger hormone” since high levels of it are found in individuals that are fasting. Ghrelin antagonistic treatments can be used to treat illnesses such as anorexia and loss of appetites in cancer patients. Ghrelin treatments for obesity are still under intense scrutiny and no conclusive evidence has been reached. This hormone stimulates growth hormone release. In yet some further embodiments the combined composition of the invention may further comprise Cholecystokinin As used herein Cholecystokinin is responsible for gall bladder secretions, gastrointestinal motility as well as pancreatic exocrine secretions. Peptide YY that may be also comprised within the composition of the invention is involved mostly in satiation modulation. Still further, the combined compositions of the invention may comprise Pancreatic polypeptide.

Pancreatic polypeptide function is most apparent in control of gastrointestinal motility and satiation. In further embodiments Amylin may be also added to the combined compositions of the invention. Amylin controls glucose homeostasis and gastric motility. Further embodiments relate to the addition of Glucose-dependent insulinotropic polypeptide to the combined compositions of the invention. Glucose-dependent insulinotropic polypeptide possesses an acute influence on food intake through its effects on adipocytes. In further embodiments, Glucagon-like peptide-1 may be added to the compositions of the invention. Glucagon-like peptide-1 has an effect on incretin activity as well as satiation. In other embodiments, Glucagon-like peptide-2 may be added to the compositions of the invention. Glucagon-like peptide-2 is responsible for gastrointestinal motility and growth. Further embodiments relate to the addition of Oxyntomodulin to the combined compositions of the invention. Oxyntomodulin plays a role in controlling acid secretion and satiation.

In yet another embodiment the composition of the invention may be administered as an add-on to a further therapeutic agent that may be an autologous protein-containing tissue extract, for example, colon or liver. Such extract comprises disease-associated antigens that modulate the immune response in the treated subject.

It is further contemplated that a composition of the present invention is used in a method for treating liver damage and/or restoring liver function in a subject in need thereof. In some embodiments, such composition may comprise as an active ingredient a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the composition of the invention may optionally further comprise at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

Specific embodiments of the above application may include disorders wherein said subject is suffering from a liver disease, said liver disease is any one of viral, bacterial, fungal or parasitic liver disease, alcoholic or autoimmune hepatitis, alcoholic or autoimmune cirrhosis, alcoholic fatty liver disease, nonalcoholic fatty liver disease (NAFLD), liver steatosis, alcoholic or nonalcoholic steatohepatits (NASH), hepatocellular carcinoma, drug-induced liver disease and pediatric liver disease and metabolic liver disease.

In further specific embodiments, a composition of the invention is used in a method for treating, preventing, ameliorating, reducing or delaying the onset of acute or chronic toxic effect of a drug and for restoring liver function.

Of particular relevance are drugs from the group of analgesics or antipyretics that have been related to adverse effects in the liver or other forms of liver damage.

The tissue-damaging effects of hyperglycemia are well known in diabetic patients, including microvascular complications (retinopathy and nephropathy), macrovascular complications (ischaemic heart disease, vascular disease, stroke and renal artery stenosis) and neuropathies. Microvascular tissue damage is the results of hyperglycaemia per se. Macrovascular complications are found to be associated with insulin-resistant states and hyperinsulinaemia. Due to these complications diabetes is also a most frequent cause of blindness and cardiovascular disease. Certain cells types are known to be vulnerable to direct damage from chronic hyperglycemia, for e.g. mesangial cells of kidney, vascular endothelial cells, pancreatic beta cells, Schwann cells and neurons.

Alcohol affects virtually every organ and tissue in the body, with multi-factorial actions on cellular and molecular functions. Alcohol itself alters biological function by direct interaction with cellular components and also due to effect of alcohol metabolism on the systemic oxidative and inflammatory state. Alcohol metabolism produces acetaldehyde and reactive oxygen (and other) species, biochemical moieties that damage healthy tissue. Oxidative stress ensuing from these reactive oxygen and nitrogen species in many organs and tissues may vary in severity depending on the systemic inflammatory and oxidative state, and on systemic and local immune function.

In specific embodiments, compositions of the present invention are particularly applicable to prevent liver and/or pancreatic tissue damage. Hazardous effects of alcohol on progressive and irreversible damage of the pancreatic (chronic pancreatitis) and liver (liver cirrhosis) tissues are well documented. There is an increased incidence of cirrhosis in diabetic patients, 80% of which have glucose intolerance.

Further, obstruction of pancreatic and liver damage by compositions of the present invention is particularly important for maintenance of glucose homeostasis, the liver being the major organ for insulin-mediated glycogen storage and the pancreas—for production of insulin and glucagon.

In this sense, compositions of the present invention are intended to prevent any condition associated with alteration of pancreatic or liver function or alteration of pancreatic or liver metabolic capacity. Those conditions may include drug-induced pancreatic and liver damage, inflammatory pancreatic and liver damage resulting from infections and autoimmune disorders, pancreatic and liver malignancies and other pancreatic and liver dysfunctions.

Still further, compositions of the present invention may be used for prevention of any target organ damage related to conditions associated with abnormal glucose homeostasis, such as pre-diabetes, diabetes, hepatic disorders, pancreatic dysfunction, diabetes, obesity, insulin resistance, metabolic disorders or any type of inflammation of the pancreas, liver, muscle or the adipose tissue.

In some specific embodiments, the invention provides a composition for use in treating and preventing pre-diabetes and diabetes. In more specific embodiments, such composition may comprise as an active ingredient at least one plant sterol, specifically CardioAid. In certain embodiments, the plant sterols, specifically, CardioAid is present in the composition of the invention in an amount sufficient for reducing blood sugar levels.

In yet some further embodiments, such composition may be an add-on to SSB in an amount sufficient for reducing and preventing the elevation in blood sugar levels associated with consumption of said SSB.

In some further embodiments, said composition may comprise in addition to plant sterols, specifically, CardioAid, at least one of soy extracts, beta-glycolipides, and different adjuvants.

In yet some further specific embodiments, the invention provides a composition for use in treating and preventing pre-diabetes and diabetes. In more specific embodiments, such composition may comprise as an active ingredient at least one lunasin peptide or a derivative thereof. In certain embodiments, lunasin peptide is present in the composition of the invention in an amount sufficient for reducing blood sugar levels.

In yet some further embodiments, such composition may be an add-on to SSB in an amount sufficient for reducing and preventing the elevation in blood sugar levels associated with consumption of said SSB. It should be noted that any of the lunasin peptides and preparation described herein before are applicable for this aspect as well.

In some further embodiments, said composition may comprise in addition to lunasin peptide or a derivative thereof, at least one of soy extracts, beta-glycolipides, and different adjuvants.

It is another important aspect of the present invention to provide a method for controlling altered blood sugar levels, altered insulin resistance and/or hepatic function, and treating an immune related disorder, treating liver damage, restoring liver function and for treating, preventing, ameliorating, reducing or delaying the onset of acute or chronic toxic effect of a drug on an organ or tissue, said method comprises providing to a subject at least one of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the method of the invention may optionally further comprise the step of further administering at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

Specific embodiments of the above described therapeutic method of the invention are detailed below.

In some embodiments, the method of the invention may involves the administration of at least one plant sterol/s. In yet a further embodiment, a therapeutic method of the invention may use as a sterol derivative or mixture of sterols, a preparation of Cardioaid™ comprising at least one of CardioAid-S, CardioAid-XF, CardioAid-SWD and CardioAid-SF.

In yet some further embodiments, the method of the invention may involve the administration of at least one lunasin peptide/s. In yet further embodiments, the therapeutic methods of the invention may use as a derivative of lunasin peptide, any one of Lunasin Reliv, LunaRichX and Relive.

In still further specific embodiment, a therapeutic method of the invention may use as an extract of a plant from the genus Moringa, an extract of Moringa oleifera.

In yet some further embodiments, the method of the invention may use in addition to at least one of natural or synthetic plant sterols, lunasin peptide/s and extract of a plant from the genus Moringa or any combinations thereof, further combinations with additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof. In one specific embodiment, a method according to the above may use as SE fraction, at least one soy-derived polar and/or non-polar fraction.

In a more specific embodiment, said therapeutic method of the invention may use a soy derived polar fraction (M1) comprising at least one of phospholipids, phosphatides or a combination thereof.

In yet another specific embodiments, said therapeutic method of the invention may use phosphatides that are any one of phosphatidylcholine (PC), phosphatidylinositol (PI) or a combination thereof, which are characteristic of M1.

In a further specific embodiment, a therapeutic method of the invention may use a soy derived non-polar fraction (OS) that comprises at least one of glycerides, phospholipids and phosphatides.

In still further specific embodiment, said therapeutic method may use at least one of glycerides, phospholipids and phosphatides that are any one of phosphatidic acid (PA), phosphatidylethanolamine (PE) and phosphatidylcholine (PC), which are characteristic of OS.

In another specific embodiment, a therapeutic method of the invention may use as a natural or synthetic beta-glycolipid, any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

In a more specific embodiment, said therapeutic method may use as glucosylceramide a beta glucosylceramide (GC).

In still further embodiment, a therapeutic method of the invention may use as a derivative of polyethoxylated castor oil is Cremophore EL (C:E).

Detailed discussion on clinical conditions which can be treated or prevented by pharmaceutical compositions of the present invention is presented further below. At this point, it should be understood that, in general, for the purpose of therapeutic applications pharmaceutical compositions are administered in an amount sufficient to cure or at least partially arrest, ameliorate, reduce or delay the onset of symptoms of a clinical condition and its complications, referred to herein as a therapeutically effective amount or dose. Amounts effective for this use will depend upon severity of the condition and the general state of a patient. Single or multiple administrations on a daily, weekly or monthly schedule can be carried out with dose levels and pattern being selected by the treating physician.

As previously mentioned, methods of the present invention are applicable not only to clinical but a range of non-clinical conditions, whereby compositions of the invention may be provided in a formulation adapted for add-on to a solid, semi-solid or liquid food, beverage, food additive, food supplement, medical food, botanical drug, drug and/or a pharmaceutical compound.

In specific embodiments, such methods involve use of compositions of the invention as add-on to food and/or beverage comprising an increased content of sugar and/or alcohol.

In particular embodiments, said methods use compositions of the invention in clinical contexts wherein sugar and/or alcohol consumption is related to altered blood sugar levels, altered insulin resistance and/or hepatic function in a treated subject.

According to certain specific embodiments, compositions of the invention are particularly suitable for oral or mucosal administration. More specifically, oral or mucosal pharmaceutical compositions of the invention are made by combining a therapeutically effective amount of at least one at least one of natural or synthetic plant sterols, lunasin peptide/s and extract of a plant from the genus Moringa or any combinations thereof and optionally, at least one of natural or synthetic SE, natural or synthetic beta-glycolipid or any derivative thereof and at least one adjuvant selected from group of polyethylene glycol, polyethoxylated castor oil; beta cyclo dextrin or a derivative thereof and optionally at least one additional therapeutic agent, with a pharmaceutically acceptable carrier.

The usefulness of an oral formulation requires that the active agent or combinations thereof according to the invention are bioavailable. Bioavailability of orally administered drugs can be affected by a number of factors, such as drug absorption throughout the gastrointestinal tract, stability of the drug in the gastrointestinal tract, and the first pass effect. Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home. Solid oral dosage forms include, but are not limited to, tablets (e.g., chewable tablets), capsules, caplets, powders, pellets, granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release. In addition, a capsule comprising the powder, pellets or granules can be further coated. A tablet or caplet can be scored to facilitate division for ease in adjusting dosage as needed.

As one example, a tablet can be prepared by compression or by molding. Compressed tablets can be prepared, e.g., by compressing, in a suitable machine, the active ingredients (in a free-flowing form such as powder or granules, optionally mixed with an excipient. Molded tablets can be made, e.g., by molding, in a suitable machine, a mixture of the powdered combined, e.g., with no inert liquid diluent.

Liquid dosage forms also allow subjects to easily take the required dose of active ingredient. Liquid preparations can be prepared as a drink, or to be administered, for example, by a naso gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid degradation, decomposition or deactivation of the active agents by the gastrointestinal system, e.g., by gastric fluid in the stomach. For example, compositions can optionally be formulated to pass through the stomach unaltered and to dissolve in the intestines, i.e., enteric coated compositions.

Compositions of the invention can be incorporated into a pharmaceutical composition suitable for oral or mucosal administration, e.g., by ingestion, inhalation, or absorption, e. g., via nasal, intranasal, pulmonary, buccal, sublingual, rectal, dermal, or vaginal administration. Such compositions can include an inert diluent or an edible carrier. For the purpose of oral therapeutic administration, the active C:E and SE compounds can be incorporated with recipients and used in solid or liquid (including gel) form. Oral compositions can also be prepared using an excipient. Pharmaceutically compatible binding agents, and/or adjuvant materials can be included as part of the composition. Oral dosage forms comprising the above described combinations are provided, wherein the dosage forms, upon oral administration, provide a therapeutically effective blood level of the combined compositions to a subject. Also provided are mucosal dosage forms comprising said combinations wherein the dosage forms, upon mucosal administration, provide a therapeutically effective blood level of the combined compositions to a subject. For the purpose of mucosal therapeutic administration, the active combined compounds can be incorporated with excipients or carriers suitable for administration by inhalation or absorption, e.g., via nasal sprays or drops, or rectal or vaginal suppositories.

The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art. Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the pharmaceutical methods known in the art. In general, pharmaceutical compositions and dosage forms are prepared by uniformly and intimately admixing the active ingredients with liquid carriers, finely divided solid carriers, or both, and then shaping the product into the desired presentation if necessary.

Although preferred administration is oral or mucosal, it should be appreciated that compositions of the invention may be also suitable for intravenous, intramuscular, subcutaneous, intraperitoneal, parenteral, transdermal, sublingual, topical administration, or any combination thereof.

It is thus conceived that the above pharmaceutical compositions, in their various formulations, are particularly applicable to treatment of certain clinical disorders, including a hepatic disorder, pancreatic dysfunction, pre-diabetes, diabetes, obesity, insulin resistance, metabolic syndrome, alcohol intoxication, alcohol withdrawal and vertigo, an inflammation of pancreas, liver, muscle or the adipose tissue, and conditions related thereto.

In some specific embodiments, the compositions of the invention may comprise an effective amount of plant sterols, specifically, CardioAid, may be particularly suitable the treatment and prevention of pre-diabetes and diabetes.

Specific pharmaceutical compositions comprising a therapeutically effective amount of at least one of natural or synthetic plant sterols, lunasin peptide/s and extract of a plant from the genus Moringa or any combinations thereof. Optionally, these compositions may further comprise at least one of soy extract/s (SE) natural or synthetic beta-glycolipid or any derivative thereof and at least one adjuvant selected from group of polyethylene glycol, polyethoxylated castor oil; beta cyclo dextrin or a derivative thereof that are particularly applicable to methods for treating, preventing, ameliorating, reducing or delaying the onset of an immune-related disorder.

In specific embodiments, therapeutic applications of pharmaceutical compositions of the invention include an inflammatory disorder, an autoimmune disorder, an infectious disease and a proliferative disorder.

Further, it is conceived that for the purpose of specific therapeutic applications, pharmaceutical compositions of the present invention further comprise at least one additional therapeutic agent. More specifically, such agent may be any one of insulin, antibodies directed to inflammatory cytokine, or antibodies such as anti TNF antibodies, statins, analgesics, chemotherapeutic agents and antibiotics.

In further embodiments, pharmaceutical compositions of the invention may optionally further comprise additional therapeutic agent, wherein said additional therapeutic agent is any one of or any type of an organism-derived antigen, including viral and or bacterial and/or fungal and/or parasitic antigens, such as any type of hepatitis B or hepatitis C derived antigens, or any type of bacterial antigens. In further embodiments, compositions of the invention may optionally further comprise additional therapeutic agent, wherein said additional therapeutic agent may be any one of autologous or allogeneic tissue derived proteins, antigens, any type of tissue derived material obtained either from the same or from different species. In further embodiments said tissue derived material or preparations may be obtained from a healthy or diseased tissue. Non-limiting examples include tumor associated tissues, blood products, tissues obtained from an individual infected with a viral or bacterial pathogen that may be combined with any composition of the invention, as described above. It is also conceived that for the purpose of specific embodiments of the compositions and methods, the combined composition of the invention may be an add-on to any type of drugs or therapeutic compounds administered orally, intravenously, intradermaly, by inhalation or intrarectaly. These combinations can be used for promoting the effect of any of the above said compounds, or for exerting an adjuvant effect, or for improving the therapeutic effect of said drug, compound, or antigen.

More specifically, pharmaceutical compositions of the invention may optionally further comprise at least one additional therapeutic agent, said additional therapeutic agent is any one of insulin, N-acetyl cysteine (NAC), thiamine (vitamin B1), a benzodiazepine or any combination thereof and a tissue derived preparation or compound.

Insulin is a peptide hormone produced by pancreatic β-cells and is central to regulating carbohydrate and fat metabolism in the body. It causes cells in the skeletal muscles and fat tissue to absorb glucose from the blood. In other words, insulin is an anabolic hormone causing cells to take up energy substrates at the times of excess. Insulin acts through a complex mechanism involving protein phosphorylation and dephosphorylation, which lead to controlled activation of glycogen synthetase and pyruvate dehydrogenase and inactivation phosphofructokinase II and hormone-sensitive lipase. Complicated control mechanism steer hormone secretion such that metabolism is constantly adjusted by hormones to meet our widely varying energy intake and expenditure, assuring a constant internal milieu. Insulin action is countered by the catabolic hormones glucagon, adrenalin, noradrenalin and growth hormone, which act primarily through cyclic AMP (cAMP) and protein kinase A.

Supplementation of exogenous insulin (most commonly injected subcutaneously) is the predominant therapy for patients with type 1 diabetes (which do not produce insulin). Medical preparations of insulin (from the major suppliers—Eli Lilly, Novo Nordisk, and Sanofi Aventis, or others) are specially prepared mixtures of insulin plus other substances including preservatives, which delay absorption of insulin, adjust the pH of the solution to reduce reactions at the injection site. Most of the medical insulin produced today is recombinant insulin, which almost completely replaced insulin obtained from animal sources (e.g. pigs and cattle). A variety of different recombinant human insulin preparations are in widespread use. Since 2003, yeast-based insulin also became available for medical use. In addition, a number of insulin analogues, which retain the hormone's glucose management functionality, have been developed. They are either absorbed rapidly in an attempt to mimic the real β-cell insulin (as with Lilly's lispro, Novo Nordisk's aspart and Sanofi Aventis' glulisine), or steadily absorbed after injection instead of having a ‘peak’ followed by a more or less rapid decline in insulin action (as with Novo Nordisk's version Insulin detemir and Sanofi Aventis's Insulin glargine), all while retaining insulin's glucose-lowering action in the human body.

The major problem with management of insulin therapy is choosing the most appropriate insulin type and dosage/timing for each diabetic patient. The commonly used types are:
fast-acting using insulin analogues aspart, lispro, and glulisine, which begin to work within 5 to 15 minutes and are active for 3 to 4 hours.
short-acting using regular insulin which begins working within 30 minutes and is active about 5 to 8 hours.
intermediate-acting using NPH insulin which begins working in 1 to 3 hours and is active 16 to 24 hours.
long acting using analogues glargine and detemir, each of which begins working within 1 to 2 hours and continue to be active, without major peaks or dips, for about 24 hours.
ultra-long acting currently only including the analogue degludec, which begins working within 30-90 minutes, and continues to be active for greater than 24 hours.
combination insulin products using either fast-acting or short-acting insulin with a longer acting insulin, typically an NPH insulin.

It must be understood that the invention encompasses the use of any insulin preparation as an additional therapeutic agent in any of the pharmaceutical compositions described herein.

Oral, intradermal, intrarectal, inhaled, intrapulmonary, or intramucosl administration of insulin or of compounds that alter insulin metabolism or that alter or potentiate its effects, whether via direct effect following systemic absorption or indirect effect following an effect on the gut associated lymphoid tissue, or any subset of cells with which they are in direct contact, can exert beneficial effect on glucose metabolism. It also has beneficial effect on the metabolic syndrome targets, such as fatty liver disease, NASH, atherosclerosis, heart disease, hyperlipidemia and diabetes.

Still further, in certain embodiments, said additional therapeutic agent may be NAC, N-acetyl cysteine (Brand names: NAC, Mucomyst, Acetadote), which has many uses in medicine. NAC is used to counteract acetaminophen (Tylenol) and carbon monoxide poisoning. It is also used for chest pain (unstable angina), bile duct blockage in infants, amyotrophic lateral sclerosis (ALS, Lou Gehrig's disease), Alzheimer's disease, allergic reactions to the anti-seizure drug phenytoin (Dilantin), and an eye infection called keratoconjunctivitis. It is also used for reducing levels of a type of cholesterol called lipoprotein (a), homocysteine levels (a possible risk factor for heart disease) and the risk of heart attack and stroke in patients with serious kidney disease. Some people use NAC for chronic bronchitis, chronic obstructive pulmonary disease (COPD), hay fever, a lung condition called fibrosing alveolitis, head and neck cancer, and lung cancer. It is also used for treating some forms of epilepsy; ear infections; complications of kidney dialysis; chronic fatigue syndrome (CFS); an autoimmune disorder called Sjogren's syndrome; preventing sports injury complications; radiation treatment; increasing immunity to flu and H1N1 (swine) flu; and for detoxifying heavy metals such as mercury, lead, and cadmium.

Specifically relevant to the present context, NAC is also used for preventing alcoholic liver damage, for protecting against environmental pollutants including carbon monoxide, chloroform, urethanes and certain herbicides; for reducing toxicity of ifosfamide and doxorubicin, drugs that are used for cancer treatment; as a hangover remedy; for preventing kidney damage due to certain X-ray dyes; and for human immunodeficiency virus (HIV).

Healthcare providers give NAC intravenously (IV) for acetaminophen overdose, acrylonitrile poisoning, amyotrophic lateral sclerosis (ALS, Lou Gehrig's disease), kidney failure in the presence of liver disease (hepatorenal syndrome), chest pain in combination with nitroglycerin, heart attack in combination with nitroglycerin and streptokinase, and for helping to prevent multi-organ failure leading to death. NAC is sometimes inhaled or delivered through a tube in the throat to treat certain lung disorders such as pneumonia, bronchitis, emphysema, cystic fibrosis, and others.

Benzodiazepines (sometimes colloquially benzo, often abbreviated BZD), are another class of relevant therapeutic agents. BZD are psychoactive drugs whose core chemical structure is the fusion of a benzene ring and a diazepine ring, the most notable example of which is Valium. BZD enhance the effect of the neurotransmitter GABA at the GABAA receptor, resulting in sedative, hypnotic (sleep-inducing), anxiolytic (anti-anxiety), euphoric, anticonvulsant, and muscle relaxant properties; also seen in the applied pharmacology of high doses of many shorter-acting BZD are amnesic-dissociative actions. These properties make BZD useful in treating anxiety, insomnia, agitation, seizures, muscle spasms, AW and as a premedication for medical or dental procedures.

Still further, in certain embodiments, the additional therapeutic agent may be an immuno-modulatory antibody being administered orally, intravenously, intrarectaly, by inhalation or intradermally. Such antibodies may include, but are not limited to, anti TNF antibodies, both chimeric or humanized, anti-integrin antibodies, or any type of antibody. These antibodies may be combined with the combined composition of the invention and/or with any of the above compounds for prevention or amelioration of toxicity or unwanted side effects of sugar, alcohol or any drug. Alternatively, these antibodies may be combined with the compositions of the invention and/or any of the compounds described above for augmenting the beneficial effects of these antibodies or of any of the compounds described herein above.

In yet other embodiments, an additional therapeutic agent may be Vitamin B 1. Vitamin B1 (also thiamine or thiamin, i.e. sulfur-containing vitamin) is a water-soluble vitamin of the B complex. Its phosphate derivatives are involved in many cellular processes. The best-characterized form is thiamine pyrophosphate (TPP), a coenzyme in the catabolism of sugars and amino acids. Thiamine is used in the biosynthesis of the neurotransmitter acetylcholine and gamma-aminobutyric acid (GABA). Vitamin B is synthesized only in bacteria, fungi, and plants, animals must obtain it from their diet, and thus, for them, it is an essential nutrient. In mammals, deficiency results in Korsakoff's syndrome, optic neuropathy and Beriberi disease that affects the peripheral nervous system (polyneuritis) and/or the cardiovascular system. Thiamine deficiency has a potentially fatal outcome if it remains untreated. In less severe cases, nonspecific signs include malaise, weight loss, irritability and confusion.

Specifically in this context, alcoholics may have thiamine deficiency due to:
inadequate nutritional intake.
active transport of thiamine into enterocytes is disturbed during acute alcohol exposure.
liver thiamine stores are reduced due to hepatic steatosis or fibrosis.
impaired thiamine utilization due to chronic alcohol consumption.
ethanol per se inhibits thiamine transport in the gastrointestinal system.

Vitamin B1 supplementation is one of the therapeutic approaches to AW syndrome. Following improved nutrition and the removal of alcohol consumption, certain impairments linked with thiamine deficiency are reversed, in particular poor brain functionality.

It is thus conceived that the compositions of the present invention are applied in various therapeutic methods for controlling altered insulin resistance and/or hepatic function.

In specific embodiments, therapeutic methods of the invention may be applied for treating liver damage and/or restoring liver function in a subject in need thereof. In some embodiments, the method comprise the step of method comprising the step of administering a therapeutically effective amount of:

I. at least one of:

(a) at least one natural or synthetic sterol or a derivative or a mixture thereof;

(b) lunasin peptide or a derivative thereof; and

(c) at least one extract of a plant from the genus Moringa.

In yet some further optional embodiments, the method of the invention may further comprise the administration of at least one of: II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and III. any combination of (a), (b) and (c) and optionally with any combination of (d), (e) and (g); or any composition comprising the same.

More specifically, said compositions are applicable to liver diseases which is any one of viral, bacterial, fungal or parasitic liver disease, alcoholic or autoimmune hepatitis, alcoholic or autoimmune cirrhosis, alcoholic fatty liver disease, nonalcoholic fatty liver disease (NAFLD), liver steatosis, alcoholic or nonalcoholic steatohepatits (NASH), hepatocellular carcinoma, drug-induced liver disease and pediatric liver disease and metabolic liver disease.

In yet some specific embodiments, the invention provides compositions comprising Moringa or any extracts or preparations or combinations thereof for treating the above-hepatic disorders, specifically, immune-hepatitis, NASH, NAFLD Ash and hepatocellular carcinoma, drug-induced liver disease and pediatric liver disease and metabolic liver disease. As shown in Example 3, Moringa preparations combined with specific soy extracts, namely, M1 and OS extracts exhibited synergistic protective effect on immune-hepatitis. Thus, the invention specifically provides combined synergistic compositions comprising an effective amount of Moringa preparations and soy extracts useful in treating any of the above hepatic disorders as well as any immune mediated or immune-associated disorder, including infectious, inflammatory, or malignant disorders.

Further, relying on the exemplified protective effects of compositions of the invention, it is conceived that these compositions will form basis for preparation of “safe drugs”. Particularly, the combined compositions comprising at least one of natural or synthetic plant sterols, lunasin peptide/s and extract of a plant from the genus Moringa or any combinations thereof and optionally, additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof and any type of therapeutic compound or food, or any ingredient will provide protection against any type of toxicity or side effect of said drugs, and against any type of target organ toxicity. In addition, such combined compositions may enhance and augment additively or synergistically, the effects of drugs or compounds. These beneficial effects may act via augmenting of the mechanism of action of or via an indirect adjuvant effect, for example by activating other pathways, cells or organs.

Thus, compositions of the invention are also applicable to methods for treating, preventing, ameliorating, reducing or delaying the onset of acute or chronic toxic effect of a drug on any body organ and for restoring liver function wherein the drug induces liver injury. For these purposes compositions of the invention may be administered concomitantly or simultaneously, the latter also include administrations in the same formulation.

More specifically, the present invention further provides pharmaceutical compositions for treating, preventing, ameliorating, reducing or delaying the onset of acute or chronic toxic effect of an analgesic or an antipyretic drug in a subject in need thereof. Moreover, pharmaceutical compositions of the invention may be used for treating and preventing any type of liver insult selected from infectious metabolic, toxic, immune, or perfusion or blood flow related hepatic injury. Pharmaceutical compositions of the invention may comprise as an active ingredient a therapeutically effective amount of at least one of natural or synthetic plant sterols, lunasin peptide/s and extract of a plant from the genus Moringa or any combinations thereof. In yet some optional embodiments such compositions may further comprise a combination of additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof, and optionally at least one additional therapeutic agent, with a pharmaceutically acceptable carrier.

In more specific embodiments said therapeutic agent may be an analgesic or an antipyretic drug, such as for example an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate.

According to one specific embodiment, pharmaceutical compositions of the invention are intended for treating, preventing, ameliorating, reducing or delaying the onset of acute or chronic toxic effect of the analgesic drug N-(4-hydroxyphenyl) ethanamide, known as acetaminophen (paracetamol).

N-(4-hydroxyphenyl) ethanamide Paracetamol or acetaminophen is a widely used over-the-counter analgesic (pain reliever) and antipyretic (fever reducer). It is commonly used non-steroidal analgesic agent for the relief of fever, headaches, and other minor aches and pains, and is a major ingredient in numerous cold and flu remedies.

While acetaminophen has fewer gastro-intestinal side effects than aspirin, another commonly used non-steroidal analgesic agent, acute and chronic acetaminophen toxicity can result in gastro-intestinal symptoms, severe liver damage, and even death. The precise intermediates in the acetaminophen toxic metabolite pathway are not yet known. As indicated herein before, it had been thought that when acetaminophen was ingested, the Cytochrom P-450 dependent enzyme system of the liver produced a potentially toxic metabolite of acetaminophen which was the cause of acetaminophen toxicity.

It was further believed that when safe amounts of acetaminophen had been ingested, this toxic metabolite was cleared by hepatic glutathione stores. However in the case of acute or chronic overdose, excessive levels of the toxic metabolite were thought to delete the glutathione stores in the liver, resulting in hepatic necrosis. Later studies have proposed that acetaminophen induced hepatic necrosis may be due to cellular oxidative stress, resulting both in lipid peroxidation, protein and non-protein thiol oxidation, and changes in the intracellular calcium homeostasis. Symptoms of acute acetaminophen toxicity are typically mild or non- existent until at least 48 hours post-ingestion.

Thus, in yet another embodiment the acute or chronic toxic effect of acetaminophen treated by the combined composition of the invention may be any one of drug induced liver injury (DILI), drug-induced acute steatosis, cytotoxic hepatocellular injury, acute liver failure (ALF), reperfusion injury, ischemic liver disease and acute cholestatic injury.

According to one specific embodiment, the pharmaceutical combined composition of the invention is particularly applicable for treating, preventing, ameliorating, reducing or delaying the onset of drug induced liver injury (DILI), caused by acetaminophen.

It should be appreciated that the different Cytochrome P-450 inducing or inhibiting drugs may lead to different hepatic injuries, and therefore, may be prevented or treated by the combined compositions of the invention. For example, chlorpromazine, phenylbutazone, halogenated anesthetic agents and sulindac may cause fever, rash and eosinophilia. Dapsone may lead to sulfone syndrome (i.e., fever, rash, anemia, and jaundice), INH (Isoniazid (Laniazid, Nydrazid), also known as isonicotinylhydrazine (INH) and halothane may cause acute viral and or bacterial and or fungal and or parasitic hepatitis, Chlorpromazine, erythromycin, amoxicillin-and clavulanic acid may lead to obstructive jaundice. Phenytoin, carbamazepine, Phenobarbital and primidone may cause anticonvulsant hypersensitivity syndrome (i.e., triad of fever, rash, and liver injury), Para-amino salicylate, phenytoin, sulfonamides, may lead to serum sickness syndrome, Clofibrate may lead to Muscular syndrome (i.e., myalgia, stiffness, weakness, elevated creatine kinase level), Procainamide may cause Antinuclear antibodies (ANAs), Gold salts, propylthiouracil, chlorpromazine and chloramphenicol may cause marrow injury. Drugs such as Amiodarone and nitrofurantoin may be lead to associated pulmonary injury and Gold salts, methoxyflurane, penicillamine, paraquat may also lead to Associated renal injury. Tetracycline may cause Fatty liver of pregnancy, Contraceptive and anabolic steroids and rifampin may cause bland jaundice, Aspirin may cause Reye syndrome, and Sodium valproate may lead to Reye like syndrome.

Still further, other acute hepatocellular injuries caused by drugs may be treated or prevented by the combined compositions of the invention. For example, acute viral and or bacterial and or fungal and or parasitic hepatitis-like picture may be caused by INH, halothane, diclofenac and troglitazone. Mononucleosis like picture may be a result of using phenytoin, sulfonamides or dapsone. Chronic hepatocellular injury may be a result of Pemoline or methyldopa. Massive necrosis may be a result of using acetaminophen, halothane or diclofenac.

Steatosis may also be a result of using different drugs, for example, Macro vesicular steatosis may be caused by Alcohol, methotrexate, corticosteroids, minocycline, nifedipine and TPN, Microvesicular steatosis may be caused by alcohol, valproic acid, tetracycline and piroxicam. Steatohepatitis may be a result of Amiodarone, nifedipine, synthetic estrogens and didanosine. Pseudoalcoholic injury may be caused by Amiodarone, Acute cholestasis maybe a result of using Amoxicillin-clavulanic acid, erythromycin and sulindac. Chronic cholestasis may be caused by Chlorpromazine, sulfamethoxazole-trimethoprim, tetracycline or ibuprofen. Granulomatous hepatitis may be a result of using Carbamazepine, allopurinol and hydralazine. Vascular injury may be caused by steroids, Neoplasia may be a result of using Contraceptives or anabolic steroids. Adenoma may be caused by steroids, Angiosarcoma may be a result of Vinyl chloride. Hepatocellular carcinoma may be caused by Anabolic steroids, aflatoxin, arsenic or vinyl chloride.

More particularly, a drug such as Amoxicillin may cause hepatic dysfunction including jaundice, hepatic cholestasis, and acute cytolytic hepatitis.

Statins are among the most widely prescribed medications in the western world. The use of statins/HMG-CoA reductase inhibitors is associated with biochemical abnormalities of liver function, and thus may be also prevented or treated by the combined composition of the invention. Moderate elevations of serum transaminase levels (<3 times the upper limit of the reference range) have been reported following initiation of therapy and are often transient. Elevations are not accompanied by any symptoms and do not require interruption of treatment. Persistent increases in serum transaminase levels (>3 times the upper limit of the reference range) occur in approximately 1% of patients, and these patients should be monitored until liver function returns to normal after drug withdrawal. Active liver disease or unexplained transaminase elevations are contraindications to use of these drugs. Patients with a recent history of liver disease or persons who regularly consume alcohol in large quantities, should use statins in a regulated manner.

In certain embodiments, the combined compositions of the invention may also be applicable for preventing and treating liver injury caused by Rifampin. Rifampin is usually administered with INH. On its own, rifampin may cause mild hepatitis, but this is usually in the context of a general hypersensitivity reaction. Fatalities associated with jaundice have occurred in patients with liver disease and in patients taking rifampin with other hepatotoxic agents. Careful monitoring of liver function (especially SGPT/SGOT) should be performed prior to therapy and then every 2-4 weeks during therapy. In some cases, hyper-bilirubinemia resulting from competition between rifampin and bilirubin for excretory pathways of the liver can occur in the early days of treatment. Isolated cholestasis also may occur.

In yet a further embodiment, the combined compositions of the invention may be applicable for preventing or treating liver damage caused by Valproic acid and divalproex sodium. More specifically, microvesicular steatosis is observed with alcohol, aspirin, valproic acid, amiodarone, piroxicam, stavudine, didanosine, nevirapine, and high doses of tetracycline. Prolonged therapy with methotrexate, INH, ticrynafen, perhexiline, enalapril, and valproic acid may lead to cirrhosis. Valproic acid typically causes microsteatosis. This drug should not be administered to patients with hepatic disease and may be used with caution in patients with a prior history of hepatic disease. Those at particular risk include children younger than 2 years, those with congenital metabolic disorders or organic brain disease, and those with seizure disorders treated with multiple anticonvulsants.

Hepatic failures resulting in fatalities have occurred in patients receiving valproic acid. These incidents usually occur during the first six months of treatment and are preceded by nonspecific symptoms such as malaise, weakness, lethargy, facial edema, anorexia, vomiting, and even loss of seizure control.

It should be further appreciated that the combined compositions of the invention may also be used for preventing or treating liver damage caused by using herbs. The increasing use of alternative medicines has led to many reports of toxicity. The spectrum of liver disease is wide with these medicines, for example: Senecio/crotalaria (Bush teas) can cause venoocclusive disease. Germander in teas is used for its anticholinergic and antiseptic properties. Jaundice with high transaminase levels may occur after two months of use, but it disappears after stopping the drug. Chaparral is used for a variety of conditions, including weight loss, cancer, and skin conditions. It may cause jaundice and fulminant hepatic failure. Chinese herbs have also been associated with hepatotoxicity.

According to certain embodiments, the compositions and combined compositions of the invention may also be applicable in treating liver damage caused by recreational drugs. More specifically, Ecstasy is an amphetamine used as a stimulant and may cause hepatitis and cirrhosis. Cocaine abuse has been associated with acute elevation of hepatic enzymes. Liver histology shows necrosis and microvascular changes.

More specifically, according to some embodiments, in addition to the enhancement or the augmentation of the beneficial effect of insulin whether via a direct or an indirect adjuvant effect, as described above, the pharmaceutical composition of the invention is intended for treating, preventing, ameliorating, reducing or delaying the onset of acute or chronic toxic effect of insulin.

According to certain specific embodiments, the composition and combined compositions of the invention is particularly suitable for oral or mucosal administration. The usefulness of an oral formulation requires that the active agent or combinations of the invention be bioavailable.

In specific embodiments, said compositions are applicable to counteract toxic effects of analgesic or antipyretic drugs given in a separate formulation without jeopardizing their beneficial therapeutic effects. More specifically, such compositions may be administered concomitantly with at least one additional therapeutic agent selected from analgesic or antipyretic drug. Such analgesic or antipyretic drug may be according to certain embodiments, an inducer or inhibitor of Cytochrom P-450 selected from the group consisting of: Acetaminophen, Phenobarbital, Phenytoin, Carbamazepine, Primidone, Ethanol, Glucocorticoids, Rifampin, Griseofulvin, Quinine, Omeprazole, Amiodarone, Cimetidine, Erythromycin, Grape fruit, Isoniazid, Ketoconazole, Metronidazole, Sulfonamides, Chlorpromazine, phenylbutazone, halogenated anesthetic agents, sulindac, Dapsone, INH, halothane, amoxicillin-clavulanic acid, phenobarbital, Para-amino salicylate, Clofibrate, Procainamide, Gold salts, propylthiouracil, chloramphenicol, nitrofurantoin, methoxyflurane, penicillamine, paraquat, Tetracycline, Contraceptive and anabolic steroids, rifampin, Aspirin and Sodium valproate. According to one specific embodiment, the invention relates to a combined composition comprising acetaminophen, thereby providing a safe preparation of acetaminophen, having reduced potential for hepatic toxicity.

In specific embodiments, therapeutic methods of the invention are implemented for the prevention or alleviation of symptoms related to a condition associated with altered blood sugar levels, altered insulin resistance and/or hepatic function, wherein said condition is any one of pre-diabetes, diabetes, obesity, hepatic disorder, pancreatic dysfunction, weight gain, alcohol intoxication, alcohol withdrawal and vertigo, any condition associated with alteration of pancreatic or liver function or tissue or organ damage, and drug-induced hepatic dysfunction.

In further specific embodiments, therapeutic methods of the invention are implemented for treating a subject suffering from a disorder associated with altered insulin resistance and/or hepatic function.

In other specific embodiments, therapeutic methods of the invention are implemented for the treatment of any one of a hepatic disorder, pancreatic dysfunction, diabetes, obesity, insulin resistance, metabolic syndrome, alcohol intoxication, alcohol withdrawal and vertigo, an inflammation of pancreas, liver, muscle or the adipose tissue, inflammatory disorder and a malignancy.

In this connection, it should be understood that the term ‘malignancy’ applies to any clinical condition that becomes progressively worse. Malignancy is most commonly used as a characteristic of cancers of various types. Malignancy refers to the features of uncontrolled growth, lack of controlled cell death (apoptosis), which are usually associated with respective changes in the genetic makeup of cells. Malignancy further refers to the metastatic or invasive potential of cancer cells, and further to their resistance to treatment, and potential recurrence of cancer cells after all detectable traces of them have been removed or destroyed.

Further in this connection, the terms treatment or prevention as used herein refers to the complete range of therapeutically positive effects of administrating to a subject including inhibition, reduction of, alleviation of, and relief from, a condition, illness, symptoms or undesired side effects thereof. These also include treatment or prevention of recurrence of a disease in response to a treatment with a non-effective, or deleterious therapeutic agent, and prevention or postponement of disease development, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms inhibition, moderation, reduction or attenuation as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with fold change values, i.e., 0.1, 0.5, 1.2, 5, etc., respectively.

It should be appreciated that the active ingredients used by the compositions and methods of the invention, specifically, natural or synthetic sterol or a derivative or a mixture thereof, lunasin peptide or a derivative thereof and at least one extract of a plant from the genus Moringa, are presented in the compositions in an amount effective for treatment and/or prevention of any of the disorders indicated herein or of any condition associated therewith.

The term prevention is interchangeable with prophylaxis in referring to significant reduction of risk of occurrence of a biological or medical event that is sought to be prevented in a tissue, a system, animal or human by a researcher, veterinarian, medical doctor or other clinician, and the term prophylactically effective amount is intended to mean that amount of a pharmaceutical composition that will achieve this goal.

Thus, it is conceived that methods using any of the above compositions of the invention are applicable for controlling blood sugar levels in a subject, treating an immune related disorder, treating liver damage, restoring liver function and for treating, preventing, ameliorating, reducing or delaying the onset of acute or chronic toxic effect of a drug on any body organs or tissues. Detailed discussion on clinical conditions that are relevant to the present invention is presented further below.

Particularly for pre-clinical applications, methods of the invention use any of the above compositions in formulations adapted for add-on to a solid, semi-solid or liquid food, beverage, food additive, food supplement, medical food, botanical drug, drug and/or a pharmaceutical compound.

In specific pre-clinical applications, methods of the invention use the above compositions as add-on to foods and/or beverages comprising an increased content of sugar and/or alcohol.

Methods using the above compositions of the invention are particularly applicable for controlling blood sugar levels in a subject, wherein said control is inhibiting increase or decrease in blood sugar levels, improving glucose tolerance or altering insulin resistance state.

In certain embodiments, methods of the present invention are applicable to the prevention or alleviation of symptoms related to a condition associated with increased or decreased blood sugar levels, wherein said condition is any one of pre-diabetes, diabetes, a hepatic disorder, pancreatic dysfunction, obesity, weight gain, alcohol intoxication, alcohol withdrawal, vertigo, and tissue or organ damage or any condition associated with alteration of pancreatic or liver function in a way that alter insulin resistance and liver metabolic capability.

The present invention is directed at treating, controlling or preventing a number of medical conditions. In general, the terms preventing, controlling and treating encompass a range of conditions, starting from prevention of the development of a disease or a symptom in a patient who may predisposed to a disease but has yet been diagnosed; further including reduction, retardation or inhibition of progression symptoms of a disease; and also alleviation of symptoms of an already existing disease, i.e. reversal of said symptoms.

Methods and compositions of the invention are specifically relevant to treating, controlling, ameliorating, or preventing body weight gain, obesity, metabolic syndrome and diabetes.

By body weight gain is meant specifically body fat gain that is maintained or decreased by applying the methods and compositions of the invention. A decrease in weight or body fat may protect against cardiovascular disease by lowering blood pressure, total cholesterol, LDL cholesterol and triglycerides, and may alleviate symptoms associated with chronic conditions such as hypertension, coronary heart disease, type 2 diabetes, osteoarthritis, sleep apnea and degenerative joint disease.

The present invention is applicable to all types of obesity, including endogenous obesity, exogenous obesity, hyper-insulin obesity, hyperplastic-hypertrophic obesity, hypertrophic obesity, hypothyroid obesity and morbid obesity. Moreover, inflammation-mediated obesity may be treated particularly effectively in accordance with the invention.

By metabolic syndrome, or syndrome X, is meant a complex multi-factorial condition accompanied by an assortment of abnormalities including hypertension, hyper-triglyceridemia, hyperglycemia, low high-density lipoprotein (HDL) cholesterol and abdominal obesity, which, among others, may lead to pro-thrombotic (e.g., elevated fibrinogen or plasminogen activator inhibitor-1 in the blood) and pro-inflammatory (e.g., elevated C-reactive protein (CRP) in the blood) conditions.

The World Health Organization (WHO) guidelines for diagnosis of metabolic syndrome are (Journal of Hypertension, Volume 17, pages 151-183, 1999):
hypertension (>140 mm Hg systolic or >90 mm Hg diastolic).
dyslipidemia, defined as elevated plasma triglycerides (150 mg/dL), and/or low high-density lipoprotein (HDL) cholesterol (<35 mg/dL in men, <39 mg/dL in women).
visceral obesity defined as a high body mass index (BMI) (30 kg/m2) and/or a high waist-to-hip ratio (>0.90 in men, >0.85 in women).
microalbuminuria (urinary albumin excretion rate of 20 g/min).

Alternatively, according to the National Cholesterol Education Program (NCEP) metabolic syndrome if at least three of the following five symptoms are present (JAMA, Volume 285, pages 2486-2497, 2001):
waist circumference >102 cm (40 in) for men or >88 cm (37 in) for women.
triglyceride level of 150 mg/dL.
HDL cholesterol level <40 mg/dL for men or <50 mg/dL for women.
blood pressure >130/85 mm Hg.
fasting glucose >110 mg/dL.

Each of the disorders associated with metabolic syndrome are risk factors in their own right, and can promote atherosclerosis, cardiovascular disease, stroke, and other adverse health consequences. However, when present together, these factors are predictive of increased risk of cardiovascular disease and stroke.

In the context of the present invention, controlling or treating metabolic syndrome using the combined compositions of the invention, is meant reducing severity and/or number of symptoms associated with this medical condition, i.e. reducing any one of elevated blood glucose, glucose intolerance, insulin resistance, elevated triglycerides, elevated LDL-cholesterol, low HDL cholesterol, elevated blood pressure, abdominal obesity, pro-inflammatory states, and pro-thrombotic states. Additionally or alternatively, it is meant reducing the risk and/or the onset of developing associated diseases, i.e. cardiovascular disease, coronary heart disease and other diseases related to plaquing of the artery walls and diabetic conditions.

Further, methods and compositions of the invention are particularly advantageous for treating, controlling and preventing diabetes or diabetic conditions, such as type 1 diabetes, type 2 diabetes, gestational diabetes, pre-diabetes, slow onset autoimmune diabetes type 1 (LADA), hyperglycemia or any type of condition or compound that expose the patient to pre diabetes or to diabetes or that alters the stage of insulin resistance. For the purposes of treatment, the diabetes may be overt, diagnosed diabetes, e.g., type 2 diabetes, or a pre-diabetic condition.

Diabetes mellitus (generally referred to herein as diabetes) is a disease that is characterized by impaired glucose regulation. Diabetes is a chronic disease that occurs when the pancreas fails to produce enough insulin or when the body cannot effectively use the insulin that is produced, resulting in an increased concentration of glucose in the blood (hyperglycemia). The WHO recognizes three main forms of diabetes mellitus: type 1, type 2, and gestational diabetes (occurring during pregnancy), which have different causes and population distributions. While, ultimately, all forms are due to the beta cells of the pancreas being unable to produce sufficient insulin to prevent hyperglycemia, the causes are different. Type 1 diabetes is usually due to autoimmune destruction of the pancreatic beta cells. Type 2 diabetes is characterized by insulin resistance in target tissues, this causes a need for abnormally high amounts of insulin and diabetes develops when the beta cells cannot meet this demand. Gestational diabetes is similar to type 2 diabetes in that it involves insulin resistance, hormones in pregnancy may cause insulin resistance in women genetically predisposed to developing this condition.

Type 1 diabetes is also recognized as insulin-dependent, juvenile, or childhood-onset diabetes; type 2 diabetes—as non-insulin-dependent or adult-onset diabetes; LADA diabetes is late autoimmune diabetes of adulthood. Additionally, intermediate conditions such as impaired glucose tolerance and impaired fasting glycemia are recognized as conditions that indicate a high risk of progressing to type 2 diabetes.

In type 1 diabetes, insulin production is absent due to autoimmune destruction of pancreatic beta-cells. There are several markers of this autoimmune destruction, detectable in body fluids and tissues, including islet cell autoantibodies, insulin autoantibodies, glutamic acid decarboxylase autoantibodies, and tyrosine phosphatase ICA512/IA-2 autoantibodies. In type 2 diabetes, comprising 90% of diabetics worldwide, insulin secretion may be inadequate, but peripheral insulin resistance is believed to be the primary defect. Type 2 diabetes is commonly, although not always, associated with obesity, a cause of insulin resistance. It should be further appreciated that the method of the invention is applicable for a subject displaying increased insulin resistance.

Type 2 diabetes is often preceded by pre-diabetes, in which blood glucose levels are higher than normal but not yet high enough to be diagnosed as diabetes. The term pre-diabetes, as used herein, is interchangeable with the terms impaired glucose tolerance or impaired fasting glucose, which are terms that refer to tests used to measure blood glucose levels.

Chronic hyperglycemia in diabetes is associated with multiple, primarily vascular complications affecting microvasculature and/or macrovasculature. These long-term complications include retinopathy (leading to focal blurring, retinal detachment, and partial or total loss of vision), nephropathy (leading to renal failure), neuropathy (leading to pain, numbness, and loss of sensation in limbs, and potentially resulting in foot ulceration and/or amputation), cardiomyopathy (leading to heart failure), and increased risk of infection. Type 2, or noninsulin-dependent diabetes mellitus (NIDDM), is associated with resistance of glucose-utilizing tissues like adipose tissue, muscle, and liver, to the physiological actions of insulin. Chronically elevated blood glucose associated with NIDDM can lead to debilitating complications including nephropathy, often necessitating dialysis or renal transplant; peripheral neuropathy; retinopathy leading to blindness; ulceration and necrosis of the lower limbs, leading to amputation; fatty liver disease, which may progress to cirrhosis; and susceptibility to coronary artery disease and myocardial infarction. By ‘prevent’ it is meant that the risk of developing of diabetes is reduced or the onset of the disease is delayed. By ‘control’ or ‘treat’ it is meant that the risk of developing associated complications is reduced and/or the onset of such complications is delayed.

Diabetic conditions that are subject to treatment with compositions and methods of the invention, and their combinations with other drugs, and with insulin, according to the methods of the present invention can be diagnosed or monitored using any of a number of assays known in the field. Examples of assays for diagnosing or categorizing an individual as diabetic or pre-diabetic or monitoring said individual include, but are not limited to, a glycosylated hemoglobin (HbA1c) test, a connecting peptide (C-peptide) test, a fasting plasma glucose (FPG) test, an oral glucose tolerance test (OGTT), and a casual plasma glucose test.

HbA1c is a biomarker that measures the amount of glycosylated hemoglobin in the blood. HbA1c designates a stable minor glycated sub fraction of hemoglobin. It is a reflection of the mean blood glucose levels during the last 6-8 weeks, and is expressed in percent (%) of total hemoglobin. Alternatively, diabetes or pre-diabetes can be diagnosed by measuring blood glucose levels using any of several known tests in the field, including a fasting plasma glucose test or an oral glucose tolerance test. Using the fasting plasma glucose (FPG) test, a patient is classified as diabetic and is subject to treatment according to the methods of the present invention if the patient has a threshold FPG greater than 125 mg/dl, and a patient is classified as pre-diabetic and is subject to treatment according to the methods of the present invention if the patient has a threshold FPG greater than 100 mg/dl but less than or equal to 125 mg/dl. Using the oral glucose tolerance test (OGTT), a patient is classified as diabetic and is subject to treatment according to the methods of the present invention if the patient has a threshold 2-hour OGTT glucose level greater than 200 mg/dl. A patient is classified as pre-diabetic and is subject to treatment according to the methods of the present invention if the patient has a threshold 2-hour OGTT glucose level greater than 140 mg/dl but less than 200 mg/dl.

C-peptide, produced from proinsulin molecules, is secreted from islet cells into the bloodstream in equimolar proportion as insulin, and is used a biomarker for beta-cell function and insulin secretion. A fasting C-peptide measurement greater than 2.0 ng/ml is indicative of high levels of insulin, while a fasting C-peptide measurement less than 0.5 ng/ml indicates insufficient insulin production.

According to another embodiment, methods according to the invention may further lead to a significant reduction in pancreatic hyperplasia and hepatic fat accumulation.

Still further, according to another embodiment, methods according to the invention may downregulate the function of macrophages while increasing foxp3+ or any other type of regulatory T cells in fat tissue or in the body, suppresses inflammatory cytokine production by adipocytes and clearly leads to a marked decrease of inflammatory cell infiltration to fat tissue of a treated subject, specifically, a subject suffering from an immune-related disorder.

More particularly, methods of the invention are intended for treatment of dyslipoproteinemia, which may include hypertriglyceridemia, hypercholesterolemia and low HDL-cholesterol, obesity, NIDDM (non-insulin dependent diabetes mellitus), IGT (impaired glucose tolerance), blood coagulability, blood fibronolysis defects and hypertension.

According to certain embodiments, the immunomodulatory composition of the invention is especially advantageous for the treatment of type 1 diabetes or diabetes mellitus, thereby preventing or reducing acute complications (e.g. hypoglycemia, ketoacidosis or nonketotic hyperosmolar coma) as well as long-term complications (e.g. cardiovascular disease, chronic renal failure, retinal damage or blindness, nerve damage and microvascular damage, which may cause impotence, poor healing wounds particularly of the feet potentially leading to gangrene and amputation).

In some specific embodiments, the invention provides methods for treating and preventing diabetes or pre-diabetes in a subject in need thereof by administering to a subject in need thereof at least one plant sterol, specifically, CardioAid or any combinations or preparations thereof, in an amount effective for treating or preventing diabetes. In some specific optional embodiments such administration may further comprise the combination of at least one of said plant sterol/s, specifically, CardioAid with at least one of lunasin peptide/s, extract of a plant from the genus Moringa or any combinations thereof, soy extract/s (SE) natural or synthetic beta-glycolipid or any derivative thereof and at least one adjuvant selected from group of polyethylene glycol, polyethoxylated castor oil; beta cyclo dextrin or a derivative thereof. In yet some embodiments, such compositions and methods using the same are applicable for treating diabetes, specifically, type II diabetes. In yet some specific and particular embodiments, methods using plant sterols or any preparations thereof, for example, CardioAid, as detailed herein, may be used for treating a subject suffering from diabetes, specifically diabetes type II, provided that said subjects are not hypercholesterolemic type II diabetic patients.

According to some embodiments, methods and compositions of the invention can be used to prevent, treat and control liver diseases and disorders including hepatitis, cirrhosis, non-alcoholic steatohepatitis (NASH) (also known as non-alcoholic fatty liver disease-NAFLD), hepatotoxicity, alcoholic steatohepatitis (ASH), drug induced liver injury (DILI) and chronic liver disease. In general, the terms ‘prevent’, ‘control’ and ‘treat’ encompass the prevention of the development of a disease or a symptom from a patient who may have a predisposition of the disease or the symptom but has not been diagnosed yet to have the disease or the symptom. The inhibition of the symptoms of a disease, encompasses inhibition or retardation of the progression thereof; and the alleviation of the symptoms of a disease, namely, regression of the disease or the symptoms, or inversion of the progression of the disease and symptoms thereof.

In further embodiments, such methods may optionally further comprises the concurrent or parallel administration of an additional therapeutic agent. In some specific embodiments, such additional therapeutic agent may be any one of insulin, N-acetyl cysteine (NAC), thiamine (vitamin B1), a benzodiazepine, any gut hormone as described above, or any combination thereof.

In yet other embodiments, said methods may be applied for treating a subject suffering from a disorder associated with increased or decreased blood sugar levels.

For specific applications of the invention, said disorder may any one of a hepatic disorder, pancreatic dysfunction, diabetes, obesity, insulin resistance, metabolic syndrome, alcohol intoxication, alcohol withdrawal, vertigo, and tissue or organ damage.

In specific embodiments, therapeutic methods of the invention are used for treating, preventing, ameliorating, reducing or delaying the onset of an immune-related disorder, said method comprising the step of administering a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the method of the invention may optionally further involves the administration of at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and III. any combination of (a), (b) and (c) and optionally with any combination of (d), (e) and (g), or any composition comprising the same.

In more specific embodiments, said therapeutic methods are applied for any one of an inflammatory disorder, an autoimmune disorder, an infectious disease and a proliferative disorder.

In this context, immune-related disorder may be any one of an inflammatory disorder, an autoimmune disorder, an infectious disease and a proliferative disorder.

Immune therapy involves the exposure of components of the immune system to various elements (cytokines, disease associated antigens and natural metabolites) to combat disease processes in which a dysregulated immune response is thought to play a role. Immune dysregulation is thought to play a major part in the pathogenesis or disease course of a great number of disease processes, including various neoplastic, inflammatory, autoimmune, infectious and genetic entities.

These disorders can be perceived as a dysbalance between pro-inflammatory (Th1) and anti-inflammatory (Th2) cytokines. Or any dysbalance of cells that control the immune system whether being a regulatory cell of any kind, antigen presenting cells, or any cells capable of altering the immune system. The way the immune system responds to foreign and self-antigens, is the result of a balance between the two subtypes of responses. Experimental autoimmune diseases in humans can be perceived as a dysbalance between pro-inflammatory and anti-inflammatory cytokines. Or a dysbalance between cells or cytokines or chemokines.

In the past few years it has been become increasingly clear that T cells capable of actively suppressing immune responses are thought to be in part responsible for the maintenance of peripheral self-tolerance. In healthy rodents and humans, there are different types of cells which are able to exert such suppressive function in vitro and in vivo Immunoregulatory cytokines such as IL-10 or TGF-β may be critical for the suppressive effect of these cells. Regulatory T cells have potential role in human autoimmune or chronic inflammatory diseases and can be used for diagnostic or therapeutic purposes.

In yet other embodiments, methods of the invention may be used for the treatment of an autoimmune disorder. Examples of autoimmune disorders include, but are not limited to, Asthma, Primary sclerosing cholangitis, Alopecia Areata, Lupus, Anlcylosing Spondylitis, Meniere's Disease, Antiphospholipid Syndrome, Mixed Connective Tissue Disease, Autoimmune Addison's Disease, Multiple Sclerosis, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Crohn's Disease, Rheumatic Fever, Discoid Lupus, Rheumatoid Arthritis, Essential Mixed, Cryoglobulinemia Sarcoidosis, Fibromyalgia, Scleroderma, Grave's Disease, Sjogren's Syndrome, Guillain-Barre, Stiff-Man Syndrome, Hashimoto's Thyroiditis, Takayasu Arteritis, Idiopathic Pulmonary Fibrosis, Temporal Arteritis/Giant Cell Arteritis, Idiopathic Thrombocytopenia Purpura (ITP), Ulcerative Colitis, IgA Nephropathy, Uveitis, Insulin Dependent Diabetes (Type I), Vasculitis, Lichen Planus, and Vitiligo. The oral combined SE and Cremophore EL compositions described herein can be administered to a subject to treat or prevent disorders associated with an abnormal or unwanted immune response associated with cell, tissue or organ transplantation, e.g., renal, hepatic, and cardiac transplantation, e.g., graft versus host disease (GVHD), or to prevent allograft rejection.

According to specific embodiments, an autoimmune disease treated by methods of the invention may be any one of rheumatoid arthritis, type 1 diabetes, type 2 diabetes, artherosclerosis, asthma, acute and chronic graft versus host disease, systemic lupus erythmatosus, scleroderma, multiple sclerosis, inflammatory bowel disease, psoriasis, uvietis, thyroiditis and immune mediated hepatitis.

According to other embodiments, methods of the invention are applicable to the treatment of Multiple Sclerosis (MS). MS is typically characterized clinically by recurrent or chronically progressive necrologic dysfunction, caused by lesions in the CNS. Pathologically, the lesions include multiple areas of demyelination affecting the brain, optic nerves, and spinal cord. The underlying etiology is uncertain, but MS is widely believed to be at least partly an autoimmune or immune-mediated disease.

According to another preferred embodiment, methods of the invention may be used for treating any inflammatory arthritis. In specific embodiments, the compositions and methods of the invention may be applicable for treating Rheumatoid arthritis (RA). RA is the most common chronic inflammatory arthritis and affects about 1% of adults, it is two to three times more prevalent in women than in men. RA may begin as early as infancy, but onset typically occurs in the fifth or sixth decade.

Diagnosis may be made according to the American Rheumatism Association Criteria for the so Classification of Rheumatoid Arthritis. A therapeutically effective amount will cause an improvement in one or more of the following: the number of inflamed joints, the extent of swelling, and the range of joint motion. Laboratory measurements (e.g., ESR and hematocrit value) and assessments of subjective features (e.g., pain and morning stiffness) can also be made.

Methods of the invention described herein can also be used to treat or prevent graft rejection in a transplant recipient. For example, methods of the invention can be used in a wide variety of tissue and organ transplant procedures, e.g., can be used to induce central tolerance in a recipient of a graft of cells, in stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail cell, tissue or organ transplantation (e.g. liver transplantation to treat hypercholesterolemia, transplantation of muscle cells to treat muscular dystrophy, or transplantation of neuronal tissue to treat Huntington's disease or Parkinson's disease).

According to another embodiment, methods of the invention may modulate the T cells or other cells balance towards a suppressing response in a subject suffering from IBD. Therefore, according to one embodiment, the composition of the invention is intended for treating IBD. Inflammatory bowel diseases (IBD) are common gastrointestinal disorders that can be perceived as being the result of a dysbalance between pro-inflammatory and anti-inflammatory subtypes of immune responses.

Patients with IBD have antibodies against components of colon cells and several different bacterial antigens. These antigens gain access to the immune system as a consequence of epithelial damage. Abnormalities of T cell-mediated immunity, including coetaneous anergy and diminished responsiveness to T cell stimuli, have also been described in these patients. In addition, changes in mucosal cell mediated immunity were identified, including increased concentrations of mucosal IgG cells and changes in T cells subsets, suggesting antigen stimulation. Exposure of target antigens after infectious, immune, or toxic damage, leads to activation of mucosal immune cells resulting in cytokines that lead to mucosal inflammatory response. Secretion of pro-inflammatory cytokines such as IFNγ, contributes to an increase in mucosal permeability, and has been described in animal models of IBD.

In other specific embodiments, methods and compositions of the invention are intended for the treatment of a malignancy. In cancerous situations, modulation of the T cell balance may be in the direction of inducing a pro-inflammatory response or in augmenting the anti-tumor associated antigens immunity. As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the compositions of the present invention may be used in the treatment of non-solid and solid tumors.

Malignancy, as contemplated in the present invention may be selected from the group consisting of carcinomas, melanomas, lymphomas, myeloma, leukemia and sarcomas. Malignancies that may find utility in the present invention can comprise but are not limited to hematological malignancies (including leukemia, lymphoma and myeloproliferative disorders), hypoplastic and aplastic anemia (both viral and or bacterial and or fungal and or parasiticly induced and idiopathic), myelodysplastic syndromes, all types of paraneoplastic syndromes (both immune mediated and idiopathic) and solid tumors (including lung, liver, breast, colon, prostate GI tract, pancreas and Karposi). More particularly, the malignant disorder may be hepaotcellular carcinoma, colon cancer, melanoma, myeloma, acute or chronic leukemia.

It should be noted that the immuno-modulatory methods and compositions of the invention may be applicable for treating infectious diseases caused by bacterial infections, viral and or bacterial and or fungal and or parasitic infections, fungal infections, or parasitic infections. More specifically, the viral and or bacterial and or fungal and or parasitic infection may be caused by any one of HBV, HCV or HIV.

In some specific embodiments, methods of the invention may be suitable for treating an immune-related disorder, for example, hepatitis.

In specific embodiments, therapeutic methods of the invention may further involve the concurrent or parallel administration of at least one additional therapeutic agent.

In more specific embodiments, such additional therapeutic agent may be any one of insulin, N-acetyl cysteine (NAC), thiamine (vitamin B1), a benzodiazepine or any combination thereof and a tissue derived preparation or compound.

In specifically relevant embodiments, said methods of the invention are implemented for treating liver damage and/or restoring liver function in a subject in need thereof, said method comprising the step of administering a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the administration may optionally include at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g), or any composition comprising the same.

In more specific embodiments, such methods may be introduced to a subject that is suffering from a liver disease, said liver disease is any one of viral, bacterial, fungal or parasitic liver disease, alcoholic or autoimmune hepatitis, alcoholic or autoimmune cirrhosis, alcoholic fatty liver disease, nonalcoholic fatty liver disease (NAFLD), liver steatosis, alcoholic (ASH) or nonalcoholic steatohepatits (NASH), hepatocellular carcinoma, drug-induced liver disease and pediatric liver disease and metabolic liver disease.

In yet other specific embodiments, therapeutic methods of the invention may be implemented for treating, preventing, ameliorating, reducing or delaying the onset of acute or chronic toxic effect of a drug and for restoring liver function.

In more specific embodiments, therapeutic methods of the invention may be applied as above to prevent, ameliorate, or reduce effects of a drug that is an analgesic or an antipyretic drug, and restore liver function.

More specifically, the present methods may apply to a subject suffering from a liver disease, that may be any one of viral and or bacterial and or fungal and or parasitic, alcoholic or autoimmune hepatitis, alcoholic or autoimmune cirrhosis, alcoholic fatty liver disease, nonalcoholic fatty liver disease (NAFLD), any type of liver steatosis, for example, due to other disease such as Wilson's disease or alpha 1 anti-trypsin deficiency, alcoholic (ASH) or nonalcoholic steatohepatits (NASH), hepatocellular carcinoma, drug-induced liver disease and pediatric liver disease and any type of metabolic liver disease, for example, glycogen storage disease

The terms liver disease or liver damage as used herein apply to many diseases and disorders that cause the liver to function improperly or to cease functioning, and this loss of liver function is indicative of liver disease. Thus, liver function tests are frequently used to diagnose liver disease. Examples of such tests include, but are not limited to, the following:
Assays to determine the levels of serum enzymes such as lactate dehydrogenase (LDH), alkaline phosphatase (ALP), aspartate aminotransferase (AST), and alanine aminotransferase (ALT), where an increase in enzyme levels indicates liver disease. One of skill in the art will reasonably understand that these enzyme assays indicate only that the liver has been damaged. They do not assess the liver's ability to function. Other tests can be used to assay a liver's ability to function.
Assays to determine serum bilirubin levels. Serum bilirubin levels are reported as total bilirubin and direct bilirubin. Normal values of total serum bilirubin are 0.1-1.0 mgdl (e.g., about 2-18 mmol/L). Normal values of direct bilirubin are 0.0-0.2 mg/dl (0-4 mmol/L). Increases in serum bilirubin are indicative of liver disease.
Assays to determine serum protein levels, for example, albumin and the globulins (e.g., alpha, beta, gamma). Normal values for total serum proteins are 6.0-8.0 g/dl (60-80 g/L). A decrease in serum albumin is indicative of liver disease. An increase in globulin is indicative of liver disease.

Other tests include prothrombin time, international normalized ratio, activated clotting time (ACT), partial thromboplastin time (PTT), prothrombin consumption time (PCT), fibrinogen, coagulation factors; alpha-fetoprotein, and alpha-fetoprotein-L3 (percent).

In some embodiments, methods of the invention may further comprise concurrent or parallel administration of at least one additional therapeutic agent.

In certain embodiments such agent is any one of insulin, antibodies directed to inflammatory cytokine, or antibodies such as anti TNF antibodies including humanized antibodies, statins, analgesics, chemotherapeutic agents and antibiotics.

In yet other embodiments, said additional therapeutic agent is any one of N-acetyl cysteine (NAC), thiamine (vitamin B1), a benzodiazepine or any combination thereof and a tissue derived preparation or compound.

In still further embodiment the additional therapeutic agent that may be an autologous protein-containing tissue extract, for example, colon or liver. Such extract comprises disease-associated antigens that modulate the immune response in the treated subject.

Further, methods of the invention, particularly those using compositions of the invention comprising a therapeutically effective amount of at least one SE or any fraction thereof and at least one polyethoxylated castor oil or any derivative thereof, or any combination thereof, are particularly applicable for treating liver damage and/or restoring liver function in a subject in need thereof.

More specifically, such methods are applicable for treating subjects suffering for example from a liver disease, said liver disease is any one of viral, bacterial, fungal or parasitic liver disease, alcoholic or autoimmune hepatitis, alcoholic or autoimmune cirrhosis, alcoholic fatty liver disease, nonalcoholic fatty liver disease (NAFLD), liver steatosis, alcoholic (ASH) or nonalcoholic steatohepatits (NASH), hepatocellular carcinoma, drug-induced liver disease and pediatric liver disease and any type metabolic liver disease, for example glycogen storage disease.

Specific embodiments of said methods are applicable for treating, preventing, ameliorating, reducing or delaying the onset of acute or chronic toxic effect of a drug and for restoring liver function.

The combined composition are presently demonstrated as ameliorating Con A induced hepatitis thereby establishing the feasibility of using the composition of the invention for treating any liver damage. One clinically important type of liver disease is hepatitis. Hepatitis is an inflammation of the liver that can be caused by viruses (e.g., hepatitis virus A, B and C (HAV, HBV, and HCV, respectively), chemicals, drugs, alcohol, inherited diseases, or the patient's own immune system (autoimmune hepatitis). This inflammation can be acute and resolve within a few weeks to months, or chronic, and persist over many years. Chronic hepatitis can persist for decades before causing significant symptoms, such as cirrhosis (scarring and loss of function), liver cancer, or death. Other important examples of the different diseases and disorders encompassed by the term “liver disease” and suitable for treatment or prevention or control using the compositions and methods of the present invention include, but are not limited to amebic liver abscess, biliary atresia, fibrosis, cirrhosis, coccidioidomycosis, delta agent, hepatocellular carcinoma (HCC), alcoholic liver disease, primary biliary cirrhosis, pyogenic liver abscess, Reye's syndrome, sclerosing cholangitis, and Wilson's disease. In some embodiments, the compositions and methods described herein are suitable for the treatment of liver disease characterized by the loss or damage of parenchymal liver cells. In some aspects, the etiology of this can be a local or systemic inflammatory response. As the ConA immune mediated hepatitis model, the beneficial effect of SE in this model forms the basis for its potential beneficial effect in any immune-related disease, in which the immune system plays a role in the pathogenesis thereof. Such immune-related diseases include infectious, inflammatory, and malignant disorders.

Liver failure occurs when large parts of the liver become damaged and the liver is no longer able to perform its normal physiological function. In some aspects, liver failure can be diagnosed using the above described assays of liver function or by a subject's symptoms. Symptoms that are associated with liver failure include, for example, one or more of the following, nausea, loss of appetite, fatigue, diarrhea, jaundice, abnormal/excessive bleeding (e.g., coagulopathy), swollen abdomen, mental disorientation or confusion (e.g., hepatic encephalopathy), sleepiness, and coma.

Chronic liver failure occurs over months to years and is most commonly caused by viruses (e.g., HBV and HCV), long-term/excessive alcohol consumption, cirrhosis, hemochromatosis, and malnutrition. Acute liver failure is the appearance of severe complications after the first signs of liver disease (e.g., jaundice) and includes a number of conditions, all of which involve severe hepatocyte injury or necrosis. In some embodiments, the compositions and methods described herein are particularly suitable for the treatment of hyperacute, acute, and subacute liver failure, fulminant hepatic failure and late onset fulminant hepatic failure, all of which are referred to herein as “acute liver failure.” Common causes for acute liver failure include, for example, viral and or bacterial and or fungal and or parasitic hepatitis, exposure to certain drugs and toxins (e.g., fluorinated hydrocarbons (e.g., trichloroethylene and tetrachloroethane), amanita phalloides (e.g., commonly found in the “death-cap mushroom”), acetaminophen (paracetamol), halothanes, sulfonamides, henytoins), cardiac-related hepatic ischemia (e.g., myocardial infarction, cardiac arrest, cardiomyopathy, and pulmonary embolism), renal failure, occlusion of hepatic venous outflow (e.g., Budd-Chiari syndrome), Wilson's disease, acute fatty liver of pregnancy, amebic abscesses, and disseminated tuberculosis.

The term hepatitis is used to describe a liver condition which implies injury to the liver characterized by the presence of inflammatory cells in the tissue of the organ. The condition can be self-limiting, healing on its own, or can progress to scarring of the liver. Hepatitis is acute when it lasts less than six months and chronic when it persists longer than six months. A group of viruses known as the hepatitis viruses cause most cases of liver damage worldwide. Hepatitis can also be due to toxins (notably alcohol), other infections or from autoimmune process. Hepatitis includes hepatitis from viral and or bacterial and or fungal and or parasitic infections, including Hepatitis A through E (A, B, C, D and E—more than 95% of viral and or bacterial and or fungal and or parasitic cause), Herpes simplex, Cytomegalovirus, Epstein-Barr virus, yellow fever virus, adenoviruses; non-viral and or bacterial and or fungal and or parasitic infections, including toxoplasma, Leptospira, Q fever, rocky mountain spotted fever, alcohol, toxins, including amanita toxin in mushrooms, carbon tetrachloride, asafetida, among others, drugs, including paracetamol, amoxycillin, antituberculosis medicines, minocycline and numerous others as described herein; ischemic hepatitis (circulatory insufficiency); pregnancy; autoimmune conditions, including Systemic Lupus Erythematosus (SLE); and non-alcoholic steatohepatitis.

Sterile inflammation is used to describe inflammation of the liver which is triggered by intracellular molecules released from dying cells that have lost integrity of their plasma membrane. This inflammation occurs in the absence of causative agents such as viruses or bacteria and alcohol. A number of intracellular molecules have been identified that can stimulate other cells to produce proinflammatory cytokines and chemokines. Such proinflammatory cellular molecules are thought to function by engaging receptors on cytokine-producing cells. If left untreated, sterile inflammation may progress to non-alcoholic fatty liver disease (NAFLD), non-alcoholic steatohepatitis (NASH) or cyrrhosis.

Non-alcoholic steatohepatitis or NASH is a condition of the liver in which inflammation is caused by a buildup of fat in the liver. NASH is part of a group of liver diseases, known as nonalcoholic fatty liver disease, in which fat builds up in the liver and sometimes causes liver damage that gets worse over time (progressive liver damage). “Non-alcoholic fatty liver disease” (NAFLD) is fatty inflammation of the liver which is not due to excessive alcohol use. It is related to insulin resistance and the metabolic syndrome, obesity, high cholesterol and triglycerides, and diabetes and may respond to treatments originally developed for other insulin resistant states (e.g. diabetes mellitus type 2), such as weight loss, metformin and thiazolidinediones. Non-alcoholic steatohepatitis (NASH) is the most extreme form of NAFLD, which is regarded as a major cause of cirrhosis of the liver of unknown cause [15].

Other factors that have been known to contribute to NASH include: surgery that shorten the intestines, the stomach, or both, such as jejunal bypass operation or biliopancreatic diversion; prolonged use of feeding tube or other method of receiving nutrition; certain drugs, including amiodarone, glucocorticoids, synthetic estrogens, and tamoxifen.

NASH is a condition that may get worse over time (called a progressive condition) and can cause scarring (fibrosis) of the liver, which leads to cirrhosis. “Cirrhosis” describes a condition in which liver cells have been replaced by scar tissue. The term “cirrhosis of the liver” or “cirrhosis” is used to describe a chronic liver disease characterized by replacement of liver tissue by fibrous scar tissue as well as regenerative nodules, leading to progressive loss of liver function. Cirrhosis is most commonly caused by fatty liver disease, including NASH, as well as alcoholism and hepatitis B and C, but also may be of unknown cause. Potentially life-threatening complications of cirrhosis are hepatic encephalopathy (confusion and coma) and bleeding from esophageal varices. Cirrhosis has historically been thought to be generally irreversible once it occurs, and historical treatment focused on preventing progression and complications. In advanced stages of cirrhosis, the only option is a liver transplant.

Each of the compounds above, specifically in the combined compositions and methods of the present invention can be used to treat, prevent or control chemical liver trauma and hepatotoxicity. Also chemical trauma or acute chemical trauma to the liver refers to serious injury which occurs to a patient over a short duration as a consequence of chemical toxicity, including drug-induced toxicity or trauma. Drug-induced acute liver trauma, including acetaminophen-induced acute liver trauma, is acute liver injury which occurs as a result or consequence of exposure to a drug (e.g., drug overdose), especially acetaminophen toxicity. Compounds according to the present invention are useful for reducing the injury to the liver which occurs from physical and chemical trauma, especially including drug-induced (drug overdose) and acetaminophen-induced acute liver trauma.

Hepatotoxocity is chemical liver trauma resulting from a hepatotoxic agent, or hepatotoxicity-inducing bioactive agent. The terms “hepatotoxic agent” and “a hepatotoxicity inducing bioactive agent” are used synonymously in context to describe compounds which often produce hepatotoxicity in patients administered such agents. Examples of hepatoxicity agents include, for example, anaesthetic agents, antiviral and or bacterial and or fungal and or parasitic agents, anti-retroviral and or bacterial and or fungal and or parasitic agents (nucleoside reverse transcriptase inhibitors and non-nucleoside reverse transcriptase inhibitors), especially anti-HIV agents, anticancer agents, organ transplant drugs (cyclosporin, tacrolimus, OKT3), antimicrobial agents (anti-TB, anti-fungal, antibiotics), anti-diabetes drugs, vitamin A derivatives, steroidal agents, especially including oral contraceptives, anabolic steroids, androgens, non-steroidal anti-inflammatory agents, anti-depressants (especially tricyclic antidepressants) glucocorticoids, natural products and herbal and alternative remedies, especially including St. John's wort.

Hepatotoxicity may manifest as triglyceride accumulation which leads to either small droplet (microvesicular) or large droplet (macrovesicular) fatty liver. There is a separate type of steatosis where phospholipid accumulation leads to a pattern similar to the diseases with inherited phospholipid metabolism defects (e.g. Tay-Sachs disease).

It must be understood that the combined compositions and methods of the invention are particularly applicable for treating any of the hepatic disorders described herein above.

In certain embodiments, the method of the invention may optionally further comprises the concurrent or parallel administration of at least one additional therapeutic agent. More specifically, such additional therapeutic agent may be any one of insulin, NAC, vitamin B1, a benzodiazepine, an anti-viral and or bacterial and or fungal and or parasitic or anti-inflammatory drug, a chemotherapeutic agent and any gut hormone. It is also conceived that for the purpose of specific embodiments, the methods, compositions and the combined compositions of the invention may be used as an add-on to any type of drugs or therapeutic compounds administered orally, intravenously, intradermaly, by inhalation or intrarectaly. Examples of such drugs or therapeutic compounds include, but are not limited to at least one of tissue derived antigens, tumor associated antigens, viral, bacterial, fungal, and parasitic derived antigens, as well as any type of organism derived antigens. The add-on composition that may comprise as an active ingredient at least one of natural or synthetic plant sterols, lunasin peptide/s and extract of a plant from the genus Moringa or any combinations thereof and optionally, further additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof according to the invention may be added to any type of tissue derived antigens obtained from a healthy or diseased subject, any type of drug or compound, any type of organism derived antigens, hormones, cytokines, therapeutic antibody, or any type of natural or non-natural therapeutic compound. The add-on composition of the invention may be used for promoting the effect of this compound, for exerting an adjuvant effect, or for improving the therapeutic effect of said therapeutic agent.

As previously mentioned, in alternative embodiments, the methods of the invention may be applicable for treating, preventing, ameliorating, reducing or delaying the onset of acute or chronic toxic effect of a drug. In specific embodiments, such drug may be an analgesic or an antipyretic drug.

It is a further aspect of the present invention to provide a soft or an alcoholic beverage, as well as food with high sugar content. In some specific embodiments, a soft or an alcoholic beverage or food with high sugar content may comprise:

I. at least one of: (a) at least one natural or synthetic sterol or a derivative or a mixture thereof;

(b) lunasin peptide or a derivative thereof; and (c) at least one extract of a plant from the genus Moringa; and at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g).

In some specific embodiments, the soft or alcoholic beverage of the invention may comprise sterol or a derivative or a mixture thereof. In certain embodiments, sterol may be at least one plant stanols and sterol and any esters or mixtures thereof. In more specific embodiments, plant stanols and sterol comprised within the beverage of the invention may be Cardioaid™ comprising at least one of CardioAid-S, CardioAid-XF, CardioAid-SWD and CardioAid-SF.

In yet some further embodiments, soft or alcoholic beverage of the invention may comprise lunasin peptide/s. More specifically, the lunasin peptide or a derivative thereof may be a peptide comprising the amino acid sequence as denoted by SEQ ID NO. 1, or any derivatives or functional fragments thereof, specifically, any one of the peptides as denoted by SEQ ID NO. 2-12. In more specific embodiments, these derivative of lunasin peptide may be any one of Lunasin Reliv, LunaRichX and Relive.

In yet some further embodiments, the soft or alcoholic beverage according to the invention may comprise extract of a plant from the genus Moringa. More specifically, such extract may be an extract of Moringa oleifera.

It should be further understood, that in addition to the at least one of natural or synthetic plant sterols, lunasin peptide/s and extract of a plant from the genus Moringa or any combinations thereof comprised in the soft or alcoholic beverage of the invention, these soft or alcoholic beverage further comprise additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof.

Thus, in specific embodiments, a soft or alcoholic beverage according to the above in addition to at least one of natural or synthetic plant sterols, lunasin peptide/s and extract of a plant from the genus Moringa or any combinations thereof, may further comprise as soy extract or any fraction thereof, a soy-derived polar (M1) or non-polar fraction (OS).

In more specific embodiments, such soft or alcoholic beverage comprising the M1 fraction, may comprise phosphatides that are characteristic thereof, namely any one of phosphatidylcholine (PC), phosphatidylinositol (PI) or a combination thereof.

In other specific embodiments, a soft or alcoholic beverage comprising the OS fraction, in addition to at least one of natural or synthetic plant sterols, lunasin peptide/s and extract of a plant from the genus Moringa or any combinations thereof, may further comprise at least one of glycerides, phospholipids and phosphatides, which are characteristic of OS.

In still further embodiments, a soft or alcoholic beverage of the invention in addition to at least one of natural or synthetic plant sterols, lunasin peptide/s and extract of a plant from the genus Moringa or any combinations thereof, may further comprise as a natural or synthetic beta-glycolipid, any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

In more specific embodiments, the soft or alcoholic beverage according to the above in addition to at least one of natural or synthetic plant sterols, lunasin peptide/s and extract of a plant from the genus Moringa or any combinations thereof, may further comprise glucosylceramide that is a beta glucosylceramide (GC).

In still further specific embodiments, a soft or alcoholic beverage according to the invention in addition to at least one of natural or synthetic plant sterols, lunasin peptide/s and extract of a plant from the genus Moringa or any combinations thereof, may further comprise a particular derivative of polyethoxylated castor oil that is Cremophore EL (C:E).

In this context, a sugar sweetened beverage (SSB) is any beverage that contains water (carbonated or still), a natural or an artificial sweetener and a natural or artificial flavor. A high sugar content may result from the presence of natural beverage ingredients (e.g. a fruit-based drink to which no additional sugar has been added) or the beverage may comprise added sugar. SSBs include for example but are not limited to fruit-based or fruit-flavored drinks, vegetable-based or vegetable flavored drinks, flavored water or sodas, energy drinks (also referred to as soft drinks), as well as milk-based beverages (for example chocolate milk, fruit-based milk drinks), coffees, teas and nonalcoholic wines and beers (in which the total alcohol content is less than 0.5% of the total volume). For the purpose of describing the invention, the terms added sugar, sugar sweetened and high sugar content are interchangeable. Risks of weight gain, obesity and diabetes which have been linked to consumption of sweetened beverages will be discussed further below.

It should be appreciated that the invention further encompasses foods with high sugar content comprising the at least one of natural or synthetic plant sterols, lunasin peptide/s and extract of a plant from the genus Moringa or any combinations thereof, and additional components that may be at least one of, soy extract/s (SE) natural or synthetic beta-glycolipid or any derivative thereof and at least one adjuvant selected from group of polyethylene glycol, polyethoxylated castor oil; beta cyclo dextrin or a derivative thereof.

The term “food with high sugar content” encompasses but is not limited to sugar-sweetened foods, namely foods which inherently comprise high sugar level, e.g. honey, syrup (e.g. maple, corn or date honey (silan), etc.), fruit- or dried fruit-based foods (for example but not limited to cereal, jams, spreads, yogurt, canned whole, chopped or minced fruit preserves and sauces), vegetable-based foods (soups, sauces, preserves, noodles, to name but few) or foods to which natural or artificial sugar or a sweetener was added prior to consumption, for example but not limited to milk-based foods (e.g. milk, condensed milk, yogurt, ice cream, milk shake, cream, cheese, etc.), candies, cereal- or granola-based foods, flour-based foods (e.g. bread, cookies, cakes, pasta, pastries, etc.), fruit- or dried fruit-based foods and vegetable-based foods.

It is yet another important aspect of the present invention is to provide a pharmaceutical composition for use in a method for prevention of liver steatosis or liver disease in a healthy subject exposed to conditions inducing a liver disease, said composition comprising as an active ingredient a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the composition of the invention may optionally further comprise at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g); said composition optionally further comprising a pharmaceutically acceptable carrier.

It should be appreciated that the composition of the invention may be used for treating, preventing and protecting from any damage caused by a therapeutic compound to any tissue or organ, and for restoring the biological function of said damaged tissue or organ.

A further important aspect of the present invention is to provide a pharmaceutical composition for use in a method for prevention of diabetes in a subject with pre diabetic condition, said composition comprising as an active ingredient a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the composition of the invention may optionally further comprise at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g); said composition optionally further comprising a pharmaceutically acceptable carrier.

It is another aspect of the present invention to provide a method for enhancing and augmenting the therapeutic effect of at least one therapeutic agent in a subject treated with said at least one therapeutic agent, the method comprises providing to a subject a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the method may optionally further comprise providing at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g); or any composition comprising the same.

In specific embodiments, the compound/s or any combinations or compositions thereof used by the method of the invention as defined in any one of (I), and optionally any one of (II) and (III), exert any one of an additive, an adjuvant or a synergistic therapeutic effect to said therapeutic compound.

Examples of such drugs or therapeutic compounds include, but are not limited to at least one of tissue derived antigens, tumor associated antigens, viral, bacterial, fungal, and parasitic derived antigens, as well as any type of organism derived antigens. Such therapeutic compound may be derived from any type of allogeneic, syngeneic or augologous tissue derived antigens obtained from a healthy or diseased subject, any type of drug or compound, any type of organism derived antigens, hormones, cytokines, therapeutic antibody, or any type of natural or non-natural therapeutic compound. The methods of the invention may be used for exerting an adjuvant effect and for promoting and improving the therapeutic effect of said therapeutic agent. It should be noted that in certain embodiments such augmenting and enhancing effect may be synergistic, additive, or adjuvant.

More specifically, according to some embodiments, in addition to the enhancement or the augmentation of the beneficial effect of a therapeutic compound or drug, e.g., insulin or any tissue or organ-derived antigen or preparation, whether via a direct or an indirect adjuvant effect, as described above, the pharmaceutical composition of the invention is intended for treating, preventing, ameliorating, reducing or delaying the onset of acute or chronic toxic effect of a therapeutic compound and drug.

In specific embodiments, the invention provides a method for enhancing and augmenting the therapeutic effect of therapeutic agent such as anyone of insulin, N-acetyl cysteine (NAC), thiamine (vitamin B1), a benzodiazepine or any combination thereof and a tissue derived preparation or compound.

In such application of the methods of the invention, the compound/s or any combinations or compositions thereof as defined in any one of (I) and optionally, any one of (II) and (III), is administered concurrently or in parallel with the administration of said therapeutic agent.

In yet another aspect, a composition of the invention is applied for use in enhancing and augmenting the therapeutic effect of at least one therapeutic agent in a subject treated with said at least one therapeutic agent, said composition comprising as an active ingredient a therapeutically effective amount of:

I. at least one of: (a) at least one natural or synthetic sterol or a derivative or a mixture thereof;

(b) lunasin peptide or a derivative thereof; and (c) at least one extract of a plant from the genus Moringa.

In certain embodiments, the composition of the invention may optionally further comprise at least one of:

II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; beta cyclo dextrin or a derivative thereof; and

III. any combination of (a), (b) and (c) and optionally with any combination of (d), (e) and (g); said composition optionally further comprising a pharmaceutically acceptable carrier.

In specific embodiments, the compositions of the invention according to the above may be used for enhancing and augmenting the therapeutic effect of any one of insulin, N-acetyl cysteine (NAC), thiamine (vitamin B1), a benzodiazepine or any combination thereof and a tissue derived preparation or compound.

It is understood that the methods of the invention involve various routes of administering the above described compositions. In some embodiments, the administration is at least one of oral, mucosal, nasal, transdermal, pulmonary, buccal or sublingual administration, or any combinations thereof. Other administration modes are also applicable, for example, subcutaneous, rectal, or parenteral (including intramuscular, intraperitoneal (IP), intravenous (IV) and intradermal) administration.

An amount adequate to accomplish this is defined as a “therapeutically effective dose.” Amounts effective for this use will depend upon the severity of the condition and the general state of the patient's own immune system, but generally range from about 0.001 to about 1000 mg/Kg body weight. Further including dosages from 0.0001 to 5000 mg and 0.01 to 2.5, specifically, 0.001, 0.002, 0.003, 0.004, 0.005, 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 1.5, 2, 2.5, 3, 3.5, 4, 4.5 and 5 mg of a combined composition. Single or multiple administrations on a daily, weekly or monthly schedule can be carried out with dose levels and pattern being selected by the treating physician.

More Specifically, the compositions of the invention may comprise an effective amount of natural or synthetic sterol, specifically CardioAid, range from about 0.00001 to about 10000 mg/Kg body weight.

In yet some further embodiments, the compositions of the invention may comprise an effective amount of lunasin peptide, ranging from about 0.00001 to about 10000 mg/Kg body weight.

In still some further embodiments, the compositions of the invention may comprise an effective amount of extract of a plant from the genus Moringa in an amount ranging from about 0.00001 to about 100000 mg/Kg body weight.

In yet some further embodiments, any of the compositions of the invention may comprise in addition to at least one of CardioAid, lunasin and Moring, also an effective amount of at least one of soy extract (SE), natural or synthetic beta-glycolipid/s and at least one adjuvant selected from group of polyethylene glycol, polyethoxylated castor oil; beta cyclo dextrin or a derivative thereof. Such effective amount may range between about 0.00001 to about 100000 mg/Kg body weight.

As noted above, according to some embodiments, the active ingredient used by the invention, specifically, at least one of: (a) at least one natural or synthetic sterol or a derivative or a mixture thereof; (b) lunasin peptide or a derivative thereof; and (c) at least one extract of a plant from the genus Moringa, may be administered with or in combination with at least one of (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; and beta cyclo dextrin or a derivative thereof and optionally, with at least one further additional therapeutic agent. The term “in combination with” such as when used in reference to a therapeutic regimen, refers to administration or two or more therapies over the course of a treatment regimen, where the therapies may be administered together or separately, and, where used in reference to drugs, may be administered in the same or different formulations, by the same or different routes, and in the same or different dosage form type.

As noted above, the present invention involves the use of different active ingredients, for example, at least one of: (a) at least one natural or synthetic sterol or a derivative or a mixture thereof; (b) lunasin peptide or a derivative thereof; and (c) at least one extract of a plant from the genus Moringa, and optionally, at least one of (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; and beta cyclo dextrin or a derivative thereof, that may be administered through different routes, dosages and combinations. More specifically, the treatment of disorders associated with increased blod sugar levels and hepatic disorders, as well as any diseases and conditions associated therewith, with a combination of active ingredients may involve separate administration of each active ingredient. Therefore, a kit providing a convenient modular format of the active ingredients of the invention required for treatment ,would allow the required flexibility in the above parameters.

Thus, in another aspect, the invention provides a kit comprising: I. at least one of: (a) at least one natural or synthetic sterol or a derivative or a mixture thereof; (b) lunasin peptide or a derivative thereof; and (c) at least one extract of a plant from the genus Moringa. In certain embodiments, the kit of the invention may further comprise at least one of: II. at least one of: (d) at least one soy extract (SE) or any fraction thereof; (e) at least one natural or synthetic beta-glycolipid or any derivative thereof; (f) at least one adjuvant selected from group of polyethylene glycol, polyethoxylated castor oil; and beta cyclo dextrin or a derivative thereof. Optionally, each of the combined ingredient is provided in a separate dosage form. The kit of the invention may facilitate combined treatment using different modes of administration for each compound as well as different duration of treatment.

In more specific embodiments, it should be appreciated that each of the multiple components of the kit may be administered simultaneously.

Alternatively, each of said multiple dosage forms may be administered sequentially in either order.

More specifically, the kits described herein can include a composition as described, or in separate multiple dosage unit forms, as an already prepared liquid topical, nasal or oral dosage form ready for administration or, alternatively, can include the composition as described as a solid pharmaceutical composition that can be reconstituted with a solvent to provide a liquid dosage form. When the kit includes a solid pharmaceutical composition that can be reconstituted with a solvent to provide a liquid dosage form (e.g., for oral administration), the kit may optionally include a reconstituting solvent. In this case, the constituting or reconstituting solvent is combined with the active ingredient to provide liquid dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

As discussed above, the invention provides different methods of treating, ameliorating preventing or delaying the onset of hepatic or any immune-related disorders in a subject in need. As used herein in the specification and in the claims section below, the term “treat” or “treating” and their derivatives includes substantially inhibiting, slowing or reversing the progression of a condition, substantially ameliorating symptoms of a condition or substantially preventing the appearance of symptoms of a condition, said condition is any one of an immune-related disorder and a hepatic disorder in a subject in need thereof.

The term “prevent” and all variations of this term is intended to mean the countering in advance of pathologic symptoms or a pathologic process progress. In this case it is understood that the composition is applied prior to the observation of clinical symptoms.

The terms “ameliorate” and “amelioration” relate to the improvement in the treated subject condition brought about by the compositions and methods according to the invention, wherein said improvement may be manifested in the forms of inhibition of pathologic processes associated with any one of an immune-related disorder and a hepatic disorder, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of an immune-related disorder or a hepatic disorder and their symptoms slowing their progress, further exacerbation or development, so as to appear later than in the absence of the treatment according to the invention.

By “subject in need” or “patient” it is meant any mammal who may be affected by the above-mentioned conditions, and to whom the treatment and diagnosis methods herein described is desired, including human, bovine, equine, canine, murine and feline subjects. Preferably, the patient is a human. Administering of the composition according to the method of the invention to the patient includes both self-administration and administration to the patient by another person.

The invention further encompasses the use of the composition and methods of the invention for treating any condition related to the conditions described above. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology described herein.

In a further aspect, the invention provides a pharmaceutical composition for use in a method for treating liver damage in a subject in need thereof. More specifically, such composition may comprise as an active ingredient a therapeutically effective amount of a polyethoxylated castor oil or any derivative or a combination thereof, and optionally further comprising a pharmaceutically acceptable carrier.

It should be appreciated that the invention further encompasses the use of the combined compositions of the invention in healthy people for prevention of liver steatosis or liver disease when exposed to conditions that possibly can induce any type of liver disease.

It should be further noted that the invention provides methods and compositions for prevention of diabetes in patients with pre diabetes.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials

Concanavalin A (ConA, MP Biomedicals, USA).

CardioAid (Swanson Health Products, Fargo, N. Dak. 58108 USA).

LunaRich X (Lunasin, Reliv International, Inc., Chesterfield, Mo. 63006-0405, USA).

Moringa (Dead Sea Moringa Labs, Israel).

Soybean Extracts

Soy extracts containing the polar (M1) and/or non-polar (OS) fractions were obtained by standard processing procedures for extracting soy oil and soy protein. M1 and OS fractions were subjected to qualitative LC-MS and ¹H-, ³¹P-NMR analyses to identify characteristic chemical profiles. Specific procedures pertaining to these methods are detailed below.

Two soy extracts were received from Solbar Israel (CHS):
OS- fraction, derived from the solvent extraction of soybeans into oil, and contains tri- and di-glycerides, free fatty acids and phosphatides;
M1- fraction which is derived from aqueous-ethanol extraction left after the solvent extraction, and contains isoflavones, sugars (oligo-, di-, mono-), and lipids (including—phosphatides, phytosterols, saponins).

The M1 (polar) fraction was obtained by standard hydro-alcoholic extraction of defatted soy milk to food soy protein. Qualitative LC-MS analysis used M1 dissolved in DMSO that was analyzed using C-18 reversed column and polar mobile phase consisting of water (modified with ammonium formate) and methanol. Qualitative ¹H-NMR analysis was carried out using different solvents to identify various constituents. M1 contained typical ratio of phosphatidylcholine (PC) and in phosphatidylinositol (PI), in declining order. More accurate ³¹P-NMR analysis showed that M1 was characterized with highly heterogeneous content of phospholipids and phosphatides. M1 was predominantly enriched in phosphatidylcholine (PC) and phosphatidylinositol (PI).

The OS (non-polar) fraction was dissolved in chloroform. The LC/MS analysis was carried out using a reversed column C-18 and non-polar mobile phase consisting of methanol and ethyl acetate. The LC/MS and NMR analyses showed mainly glycerides and phospholipids, in declining order. More accurate quantitative ³¹P-NMR spectroscopy showed that OS was mainly enriched in phosphatidic acid (PA), phosphatidylethanolamine (PE) and phosphatidylcholine (PC). OS and M1 fractions were distinct by ratios of various phosphatides.

Animals

Male C57BL/6 mice (10-12 weeks old) were obtained from Harlan Laboratories (Jerusalem, Israel) and maintained in the Animal Core of the Hadassah-Hebrew University Medical School. All animal experiments were carried out according to the guidelines of the Hebrew University-Hadassah Institutional Committee for the Care and Use of Laboratory Animals and with the committee's approval.

Experimental Procedures

High Fat Diet (HFD) Challenge

Mice were fed high fat diet (Harlan, TD88137 comprising 42% calories from fat) and water ad libitum starting at the age of 10 weeks (day #0), in a liberal, restriction-free manner. Mice were kept on a 12 hour light/dark cycle. In addition the mice were orally fed, by gavage, three times a week for 25 weeks with the indicated agent (CardioAid or Lunasin).

High Sugar Drinks Challenge

Chocolate Drink

All mice were administered standard laboratory chow and water ad libitum and kept in a 12-hour light/dark cycle.

Mice were administered by gavage with chocolate drink using a commercially available sweetened chocolate drink (prepared by mixing 400 DDW with 400 μl chocolate solution prepared by mixing 20 mg sweetened chocolate drink in 400 DDW) with and without CardioAid following on overnight fast. All animals were followed by measurements of serum blood glucose for three hours on the following time points: 0, 15 min, 30, 60, 90, 120, 180. Experimental groups are described in the Examples (Table I).

High Sugar Soda

C57B1, males 11-12 old, 2 mice/group were fasted overnight followed by oral administration of high sugar containing carbonated drink. Mice underwent serum glucose measurements at the following time points: 0, 15, 30, 45, 60, 90, 120 min.

The following adjuvants were added to CardioAid for assessment of sugar protective effect: Cremophor; Dextrin; OS soy extract; M1 soy extract; and PEG. Experimental groups are indicated in the Examples section (Table 3).

Concanavalin A (ConA) Challenge

All mice were administered standard laboratory chow and water ad libitum and kept in a 12-hour light/dark cycle.

Combination of Moringa and Soy Extracts—the ConA Model

Mice were treated on days 1 and 2 with Moringa and soybean extracts (OS, M1) as indicated in Examples (Table 5). Moringa and soybean extracts were prepared as detailed below. At day 2 mice were intravenously (iv) injected with 200 μl Con A solution (0.5 mg per mouse prepared by mixing 2.5 mg Con A with 1 ml distilled water).

Prior to ConA injection, mice were treated on days 1 and 2 with Moringa and soy extracts according to the following dosages.
Moringa: 3 mg/day/mouse

Moringa and OS solution was prepared from 10 mg Moringa and 10 mg OS in 2 ml OS (10 mg OS in 300 μl absolute EtOH, to which 10 mg Moringa in 100 μl Cremophor and 1.6 ml PBS were added). Each mouse was administered with 200 μL by gavage (1 mg/day/mouse of Moringa/OS).

Moringa and M1 solution was prepared from 10 mg Moringa and 10 mg M1 in 2 ml M1 (10 mg M1 in 300 μl absolute EtOH, to which 10 mg Moringa in 100 μl. Cremophor, and 1.6 ml PBS were added). Each mouse was administered with 200 μL by gavage (1 mg/day/mouse of Moringa/M1).

Mice were tested for serum alanine transaminase (ALT) at 24 hours after Con A administration. Serum ALT levels were measured by an automatic analyzer.

Induction of Nonalcoholic Steatohepatitis (NASH)

The mice were fed, starting at the age of 10 weeks (day #0), in a liberal, restriction-free, commercially available HFD (Harlan, TD88137; 42% of the calories are from fat), until their sacrifice after 25 weeks. Mice were weighted weekly.

Measuring the Effect of CardioAid and Lunasin on HFD Mice

Three groups of mice, n=6 each, were orally fed, by gavage, three times a week for 25 weeks as indicated below: Group A (Control) did not received any treatment.; Group B (CardioAid) received 25 μL of commercially available CardioAid suspended in DDW, containing 3 mg; Group C (LunaRichX) received 50 μL of commercially available LunaRichX Reliv containing 3 mg suspended in double-distilled water (DDW), per mouse per feed. The effect of the treatment on the systemic immune system was determined by FACS analysis and by measuring the levels of serum cytokines. Liver enzymes levels, liver histology, total body fat, liver triglycerides and glucose levels were also analyzed in treated mice as described below.

Isolation of Splenocytes

Spleens were obtained from sacrificed mice and were kept in RPMI-1640 supplemented with fetal calf serum (FCS). Spleens were crushed through a 70 μm nylon cell strainer and centrifuged (1250 rpm for 7 min) to remove debris. Red blood cells were lysed with 1 ml of cold 155 mM ammonium chloride lysis buffer and immediately centrifuged (1250 rpm for 3 min). The splenocytes were then washed and suspended in 1 ml of FACS buffer. Any remaining connective tissue was removed. The viability, as assessed using trypan blue staining, was above 90%.

FACS Analysis

Flow cytometry was performed on splenocytes lymphocytes with antibodies for CD4, CD8, (eBioscience, San Diego, Calif., USA) epitopes using the LSR-II. Analysis was performed using FSC express software.

Cytokine Measurement

Serum Interleukin 1-α(IL-1α), levels were measured in each animal using Custom Q-plex-10plex ELISA-based Chemiluminescent assay (Quansys Biosciences, Logan, Utah, USA), according to manufacturer's instructions. Transforming Growth Factor beta (TGFβ) level was measured in each animal using Quantikine ELISA Mouse/Rat/Porcine/Canine TGF-b1 (R&D Systems, Minneapolis, Minn., USA. Cat No MB100B), according to manufacturer's instructions.

Biochemistry Analysis

Blood was collected from individual mice at euthanasia and serum Aspartate aminotransferase (AST), Alanine aminotransferase (ALT) and gammaglutamyl transferase (gGT) levels were determined using Reflotvet Plus (Roche). Serum triglyceride (TG) were measured using the Cobas®C 111 analyzer (Roche, Switzerland). Tail-end venous blood glucose levels were measured bi-weekly using Accu-Check Performa Tests (Roche).

Serum Glucose Levels

Using tail-end venous blood sampling, serum levels of glucose were measured bi-weekly using a standard kit (Accu-Check Performa Tests).

In-Vivo Evaluation of Total Body Fat Content

The total body fat content was evaluated in-vivo by using EchoMRI™-100H (EchoMRI, Houston, Tex., USA) at week 25, prior to sacrifice.

Histological Examination of the Liver

Paraffin-embedded liver sections were prepared from each mouse. The livers were cut into 4-5 μm thin slices and stained with hematoxylin-eosin (H&E). A blinded pathologist examined the tissues using light microscopy to score for morphological and histopathological changes that are characteristic of NASH. The maximal score for steatosis (=3) was assigned for greater than 66%. The maximal score for lobular inflammation (=3) was assigned for >4 foci/200×, and hepatocyte ballooning (=2) was assigned for many cells/prominent ballooning. The maximal NAFLD Activity Score (NAS) score is a simple arithmetic combination of all three features (min. 0, max. 8).

Hepatic Triglyceride (hTG) Content

Accumulation of intracellular TGs within the liver was quantified using a modification of the Folch method (Folch J, Lees M, Sloane Stanley G H. A simple method for the isolation and purification of total lipides from animal tissues. J Biol Chem 1957; 226:497-509). Human TGs were extracted from aliquots of snap-frozen livers and then assayed using a GPO-Trinder kit (Sigma, Israel), and the levels were normalized to per gram of liver tissue in the homogenate.

Statistical Analysis

Statistical analysis was performed using a two tails Student's t-test (Using Microsoft® Excel). A p value less than 0.05 was considered significant.

### Example 1

The Effect of Administering Plant Sterols (CardioAid) and Lunasin on HFD Mice

A high-fat diet (HFD) is widely used to produce hepatic steatosis and nonalcoholic steatohepatitis (NASH) in experimental animals NASH was induced by feeding mice, starting at the age of 10 weeks with HFD, as detailed above. In addition, mice were administered with CardioAid and Lunasin as described above.

Briefly, three groups of mice were examined (n=6 in each group). The mice were orally fed (in addition to the HFD), by gavage, three times a week for 25 weeks as indicated below: Group A did not received any treatment and served as a control; Group B received CardioAid (3 mg/mouse/feed) and Group C received Lunasin (LunaRichX, 3 mg/mouse/feed). The effect of the treatment on the systemic immune system was determined by FACS analysis and by measuring the levels of serum cytokines. In addition, liver enzymes levels, liver histology, total body fat, liver triglycerides and glucose levels were analyzed as described above.

Effect of the Treatment on the Immune System

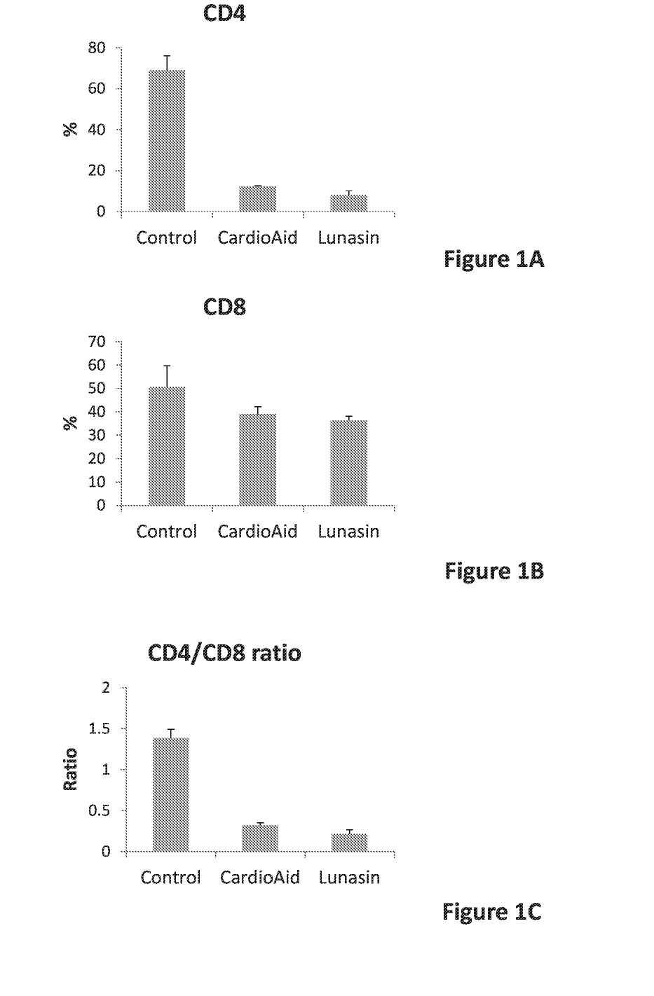

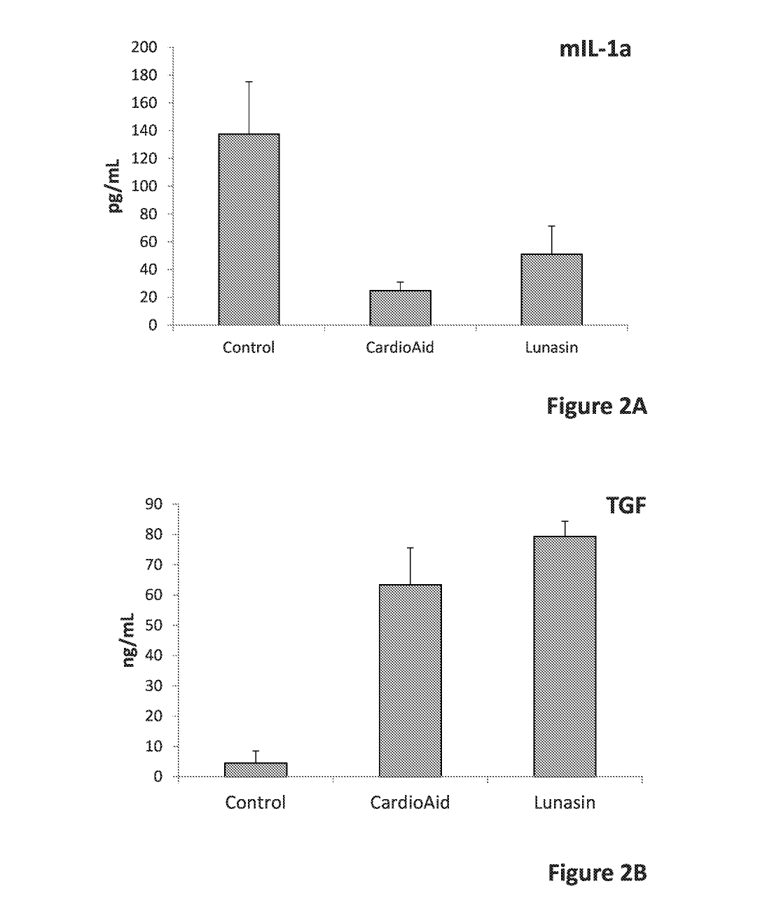

Using the HDF model described above, the immunomodulatory effect of oral administration of the tested compounds CardioAid and LunaRichX on the immune system was examined and is shown in FIG. 1 and FIG. 2. Subsets of T lymphocytes were analyzed by FACS in all experimental groups.

In particular, a decrease in the levels of CD4 and CD8 positive cells was observed for both agents, as evident from FIG. 1A and FIG. 1B, respectively. In addition, as demonstrated in FIG. 1C, a significant decrease in the CD4/CD8 ratio was noted (from 1.38 to 0.32 and 0.21, in controls vs. CardioAid and LunaRichX, respectively, p value<0.05).

FIG. 2 shows the effect of treatment on serum cytokines profile. As demonstrated in FIG. 2A, a significant decrease in IL1α was noted (From 137.4 pg/mL to 24.8 pg/mL and 51.0 pg/mL, in controls vs. CardioAid and LunaRichX, respectively, p value<0.05 for CardioAid), along with a significant increase in TGFβ serum levels shown in FIG. 2B (From 4.4 ng/mL to 63.4 ng/mL and 79.3 ng/mL, in controls vs. CardioAid and LunaRichX, respectively, p values<0.001). Overall, the data suggests an immunomodulatory effect of both compounds on the systemic immune system.

The Effect of the Treatment on Liver Damage

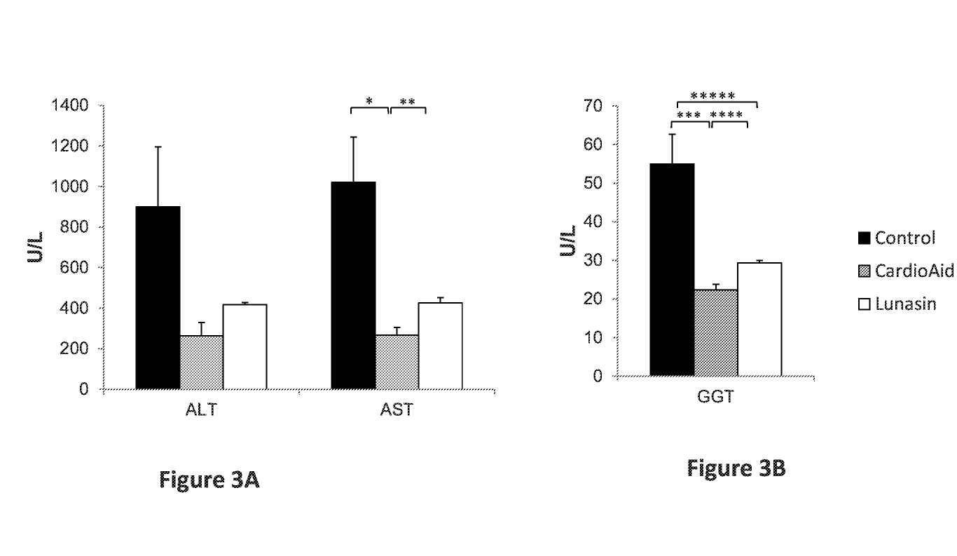

The effect of CardioAid and LunaRichX on the immune system was also associated with a liver protection effect. FIG. 3A and FIG. 3B show the effect of treatment on several serum liver enzymes levels (ALT and AST in FIG. 3A and GGT in FIG. 3B). Both treated groups showed a decreased serum liver enzymes levels, hepatocellular and Cholestatic. ALT was decreased from 900 U/L for the control group to 264 U/L for CardioAid treated group and 417 U/L for LunaRichX treated group (p value NS). A similar decrease was noted at AST levels (1021 U/L, 268 U/L and 426 U/L, respectively. P value<0.05 for CardioAid) and GGT levels (55 U/L, 22 U/L and 29 U/L, respectively. both p values<0.05).

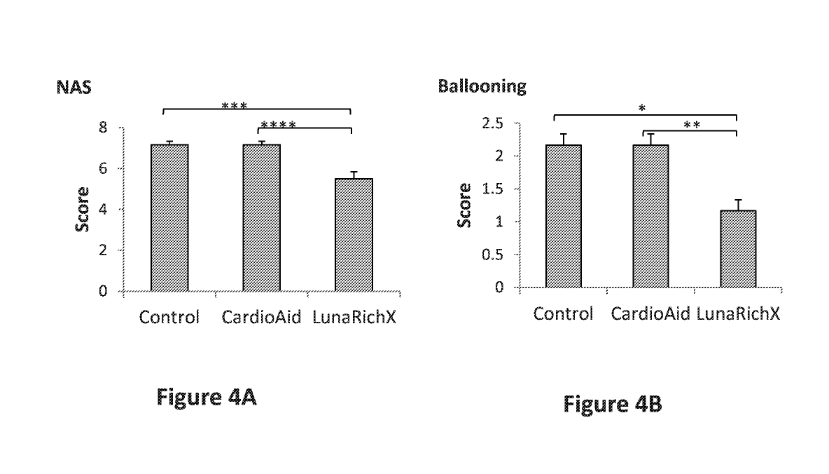

In addition, FIG. 4 shows the effect of treatment on liver histology, where a significant improvement in the NAS score, which is a quantitative score for NASH, and in the ballooning, which is the hisotological hallmark for NASH was seen in the LunaRichX-treated mice (as shown in FIG. 4A and FIG. 4B, respectively).

The Effect of the Treatment on Metabolic Parameters

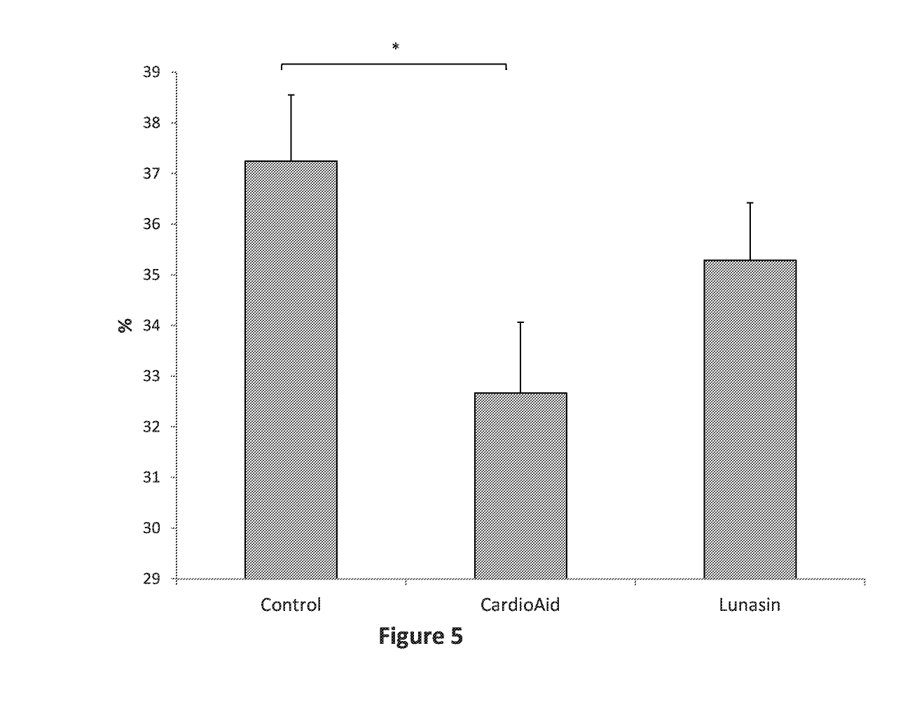

FIG. 5 shows the effect of treatment on total body fat (%) as measured by EchoMRI.mA, as detailed above. As evident from FIG. 5, decrease from 37.2% in the control group to 32.7% in the CardioAid-treated group (p value<0.05) and to 35.3% in the LunaRichX-treated group were observed.

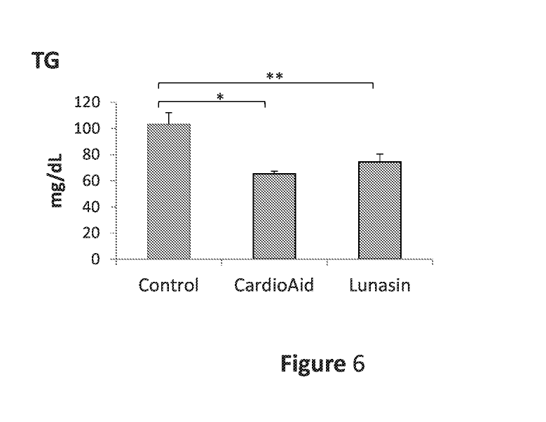

In addition, FIG. 6 shows the effect of treatment on triglycerides (TG). As demonstrated in FIG. 6, a significant decrease was noted at the serum TG levels, from 103 mg/dL in the control group to 65 mg/dL in the CartioAid treated group (p value<0.05) and to 75 mg/dL in the LunaRichX treated group.

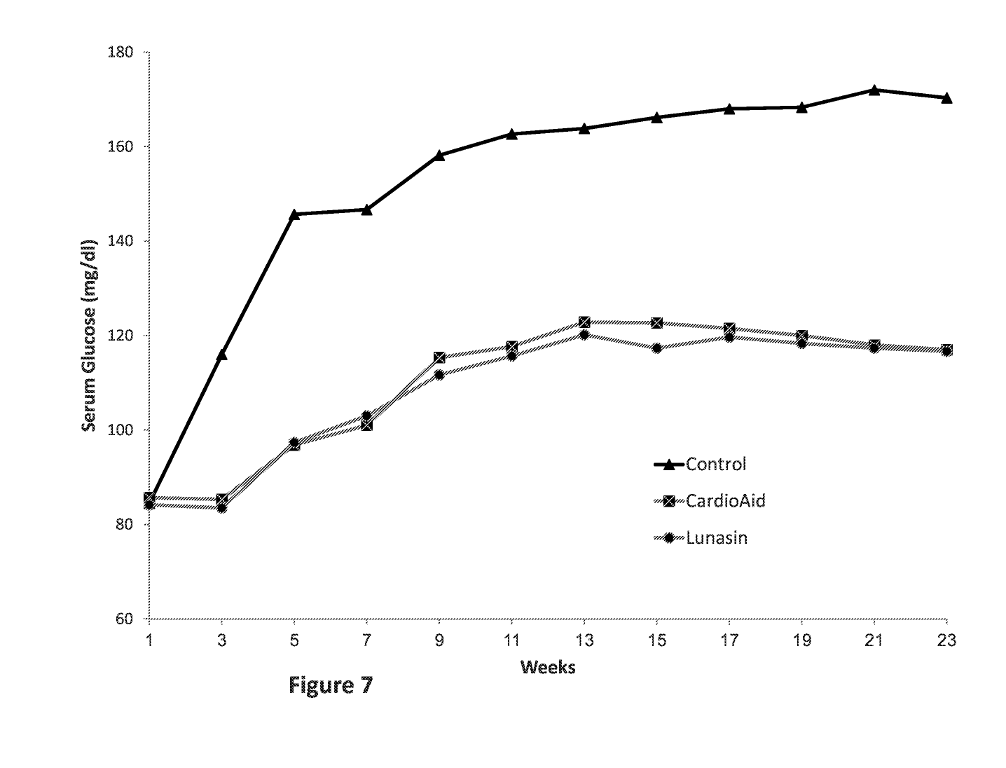

Finally, FIG. 7 shows the effect of treatment on serum glucose levels. As shown therein, starting from week 3, a significant reduction at serum glucose level (relative to the serum glucose level in the control mice) was noted for both treatments, namely CardioAid and LunaRichX. For example, at week 23 the serum glucose level in the control group was 170.3 mg/dL, wherein it was only 117 mg/dL in the CardioAid treated group and 116.6 mg/dL in the LunaRichX treated group (both p values<1×10⁻¹⁰).

Taken together the above experiments suggest a beneficial therapeutic effect for CardioAid and Lunasin on the liver.

### Example 2

Administration of CardioAid Protects Against the Increase of Serum Sugar Levels when Added to High-Sugar Beverages

Chocolate Drink

The effect of CardioAid administered with a high sugar soft drink on the serum glucose level was examined in mice, as detailed above. Briefly, male C57B1/6 mice (11-12 weeks old) were obtained from Harlan Laboratories (Jerusalem, Israel) and were administered standard laboratory chow and water ad libitum.

Mice were divided into the following experimental and control groups:

TABLE 1
experimental groups

| Group | Treatment | Quantity/mouse |
|---|---|---|
| A | Chocolate drink + DDW 1:1 | 250 μl |
| B | Choco-DDW + CardioAid | 250 μl + 1 mg |

Mice were administered with a chocolate drink prepared as described above with or without CardioAid, following on overnight fast. All animals were followed-up by measurements of serum blood glucose for three hours on the following time points: 0, 15 min, 30, 60, 90, 120, 180.

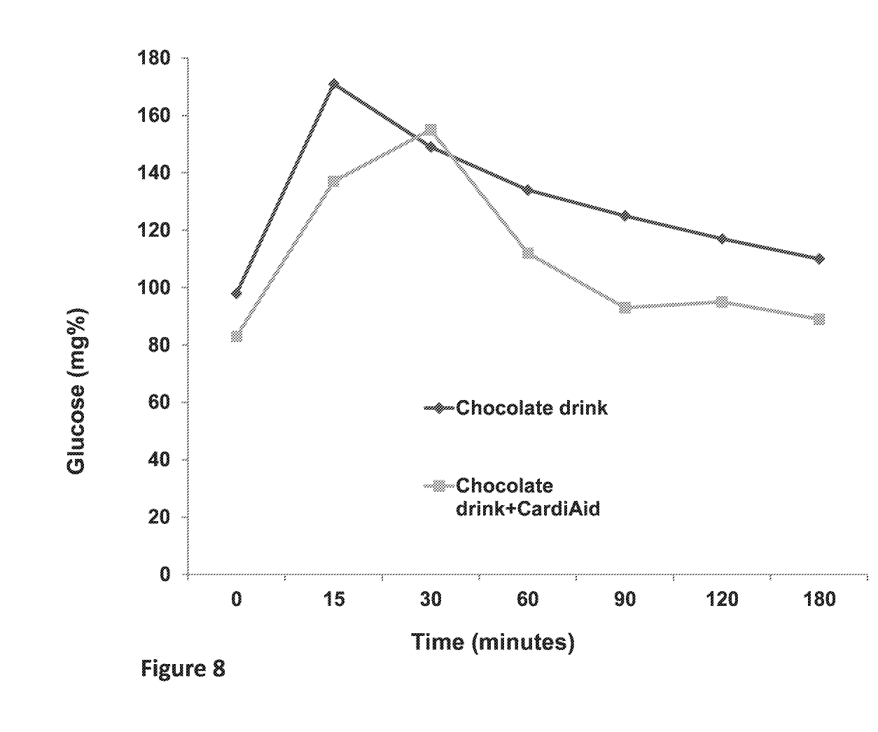

As demonstrated in FIG. 8, co-administration of CardioAid with a high sugar containing chocolate drink alleviated the increase in sugar blood levels. In addition, Table 2 below shows the glucose blood levels in mg/dL at different time points following the co administration of CardioAid with high sugar containing chocolate drink.

TABLE 2
Glucose blood levels following co administration of
CardioAid and a high sugar containing chocolate drink

| | Time (minutes) | | | | | | |
|---|---|---|---|---|---|---|---|
| | 0 | 15 | 30 | 60 | 90 | 120 | 180 |
| Chocolate drink | 98 | 171 | 149 | 134 | 125 | 117 | 110 |
| Chocolate drink + | 83 | 137 | 155 | 112 | 93 | 95 | 89 |
| CardioAid | | | | | | | |

The above demonstrates that mixing CardioAid with soft drinks containing sugar may alleviate the sugar burden assigning a protective effect for CardioAid.

High Sugar Soda

The inventors have next examined the protective effect of CardioAid on additional sugar sweetened beverages, specifically soda. Five adjuvants were tested with CardioAid: Cremophor; Dextrin; OS soy extract; M1 soy extract; and PEG, as indicated by the Experimental groups listed in Table 3. Mice were administered with a cola drink prepared as described above with or without CardioAid and different combinations thereof, following on overnight fast. All animals were followed-up by measurements of serum blood glucose for three hours on the following time points: 0, 15 min, 30, 60, 90, 120, 180.

TABLE 3
experimental groups:

| | | CA: 30 ul | |
|---|---|---|---|
| No | | PO | Cola solution |
| A1 | Cola | — | 300 ul |
| B | Cola + Cardio-Aid | 6.25 microgr | ″ |
| C | Cola + Cremophor | 250 ul in 2 ml | ″ |
| D | Cola + Dextrin | — | ″ |
| E | Cola + CA + Crem | 6.25 microgr | ″ |
| F | Cola + CA + Dextrin | 6.25 microgr | ″ |
| G2 | Cola + PEG | 250 ul in 2 ml | ″ |
| H | Cola + OS | 1 mg/mouse | ″ |
| I | Cola + M1 | 1 mg/mouse | ″ |
| J | Cola + CA + PEG | 6.25 microgr | ″ |
| K | Cola + CA + OS | 6.25 microgr | ″ |
| L | Cola + CA + M1 | 6.25 microgr | ″ |

As shown in Table 4, a synergistic effect between CardioAid and all five adjuvants tested for reduction of sugar levels was shown: Cremophor; Dextrin; M1 soy extract; OS soy extract; and PEG.

Moe specifically, the results show the increase in blood sugar levels between time 0 and 15 minutes and from 0 to 30 minutes after drinking of Cola showing a synergism between CardioAid and all five adjuvants.
For the combination of CardioAid with Cremohpor or with dextrin a synergism was shown for both the first 15 and first 30 minutes
For the combination of CardioAid with PEG, or OS, a synergism was shown for 30 minutes.
For the combination of CaridoAid and M1 a synergism was shown for the first 15 minutes.

TABLE 4
A synergistic effect between CardioAid and adjuvants in
reducing blood sugar levels following administration of soft drinks.

| | | Increase in | Increase in |
|---|---|---|---|
| | | blood sugar | blood sugar |
| | | from 0 to 15 | from 0 to 30 |
| Group | Treatment | minutes | minutes |
| A | Cola | 138 | 80.5 |
| B | Cola + Cardio-Aid | 100 | 58.5 |
| C | Cola + Cremophor | 45.5 | 19.5 |
| D | Cola + Dextrin | 102 | 67 |
| E | Cola + CA + Crem | 81.5 | 29 |
| F | Cola + CA + Dextrin | 90.5 | 19.5 |
| G | Cola + PEG | 76 | −38.5 |
| H | Cola + OS | 76 | −34 |
| I | Cola + M1 | 128.5 | −63 |
| J | Cola + CA + PEG | 122 | −46 |
| K | Cola + CA + OS | 103 | −39.5 |
| L | Cola + CA + M1 | 103 | −37 |

As shown above, addition of CardioAid to soft drinks, either alone or in combination with different additional compounds and adjuvants, clearly reduce the elevation in blood sugar levels caused by consumption of SSB. Moreover, as shown in Table 4, the protective effect of CardioAid has been synergistically enhanced when different combinations of CardioAid were used.

These results clearly establish the feasibility of using CardioAid and different combinations thereof in soft drinks as well as in reducing blood sugar levels in patients in need.

### Example 3

The Effect of Moringa and Combinations Thereof with Soybean Extracts on the Liver

Moringa is a plant native to the sub-Himalayan areas of India, Pakistan, Bangladesh, and Afghanistan and is also grown in many other areas of the world. As indicated herein before, the leaves, bark, flowers, fruit, seeds, and root are used to prepare different extracts applicable in treating different conditions. The Soybean extracts were previously shown by the inventors as performing hepatoprotective effect. The inventors therefore examined the hepatoprotective effect of Moringa, and the potential effect of combinations thereof with different soy extracts. To examine the hepatoprotective effect of Moringa preparations on liver, the inventors used mice model of autoimmune hepatitis induced by injecting Concavalin A (ConA).

Mice were treated on days 1 and 2 with Moringa and soy extracts as specified in Table 5.

TABLE 5
Experimental and control groups:

| Group | Treatment: PO | Sacrifice |
|---|---|---|
| A | Control: 40 μl Cremophor | 14 h after ConA |
| B | 3 mg OS | 14 h after ConA |
| C | 3 mg M1 | 14 h after ConA |
| D | Moringa 3 mg | 14 h after ConA |
| E | 3 mg OS + Moringa 3 mg | 14 h after ConA |
| F | 3 mg M1 + Moringa 3 mg | 14 h after ConA |

The hepato-protective effect was tested for serum alanine transaminase (ALT) at 24 hours after Con A administration. Serum ALT levels were measured by an automatic analyzer.

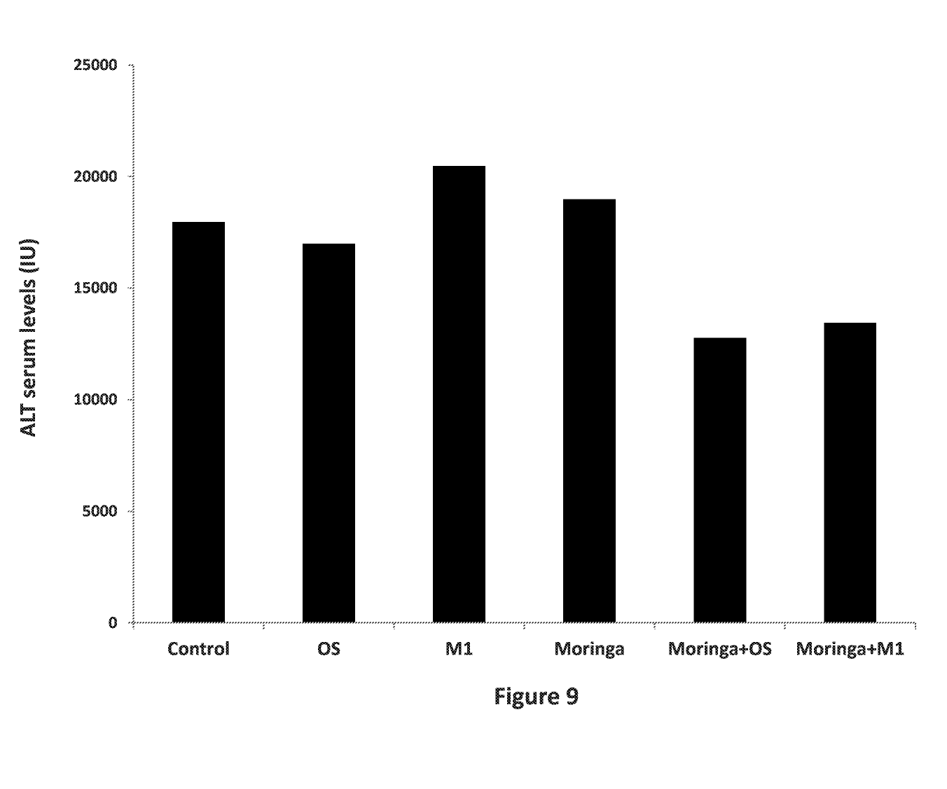

A synergistic effect between Moringa and OS or M1 was observed for alleviation of immune-mediated hepatitis, as evident from the results presented in Table 6 below, and also presented by FIG. 9.

TABLE 6
ALT levels in Con A mice treated with Moringa combinations

| | Mean ALT serum levels |
|---|---|
| Control | 17965 |
| OS | 16990 |
| M1 | 20475 |
| Moringa | 18975 |
| Moringa + OS | 12764 |
| Moringa + M1 | 13440 |

The above results suggest a beneficial effect for co-administration of Moringa with OS or with M1 soybean extracts on of serum ALT levels.

1. A composition for use in at least one of, a method for controlling blood sugar levels, altered insulin resistance and/or hepatic function in a subject, a method for the treatment of an immune related disorder, a method of treating liver damage and restoring liver function, a method for treating, preventing, ameliorating, reducing or delaying the onset of acute or chronic toxic effect of a drug, and a method for enhancing and augmenting the therapeutic effect of a therapeutic agent in a subject treated with said agent, said composition comprising as an active ingredient:
I. at least one of:
(a) at least one natural or synthetic sterol or a derivative or a mixture thereof;
(b) lunasin peptide or a derivative thereof; and
(b) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g).

2. The composition according to claim 1, wherein said sterol or a derivative or a mixture thereof is at least one plant stanols and sterol and any esters or mixtures thereof.

3. The composition according to claim 2, wherein said plant stanols and sterol is Cardioaid™ comprising at least one of CardioAid-S, CardioAid-XF, CardioAid-SWD and CardioAid-SF.

4. The composition according to claim 1, wherein said lunasin peptide or a derivative thereof is a peptide comprising the amino acid sequence as denoted by SEQ ID NO. 1, or any derivatives or functional fragments thereof.

5. The composition according to claim 4, wherein said derivative of lunasin peptide is any one of Lunasin Reliv, LunaRichX and Relive.

6. The composition according to claim 1, wherein said extract of a plant from the genus Moringa is an extract of Moringa oleifera.

7. The composition according to claim 1, wherein said SE or any fraction thereof is a soy derived polar and/or nonpolar fraction.

8. The composition according to claim 7, wherein said soy derived polar fraction comprises at least one of phospholipids, phosphatides or a combination thereof.

9. The composition according to claim 8, wherein said phosphatides are any one of phosphatidylcholine (PC), phosphatidylinositol (PI) or a combination thereof, said polar fraction is designated M1.

10. The composition according to claim 7, wherein said soy derived non-polar fraction comprises at least one of glycerides, phospholipids and phosphatides.

11. The composition according to claim 10, wherein said at least one of glycerides, phospholipids and phosphatides, which are any one of phosphatidic acid (PA), phosphatidylethanolamine (PE) and phosphatidylcholine (PC), said non-polar fraction is designated OS.

12. The composition according to claim 1, wherein said natural or synthetic beta-glycolipid is any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

13. The composition according to claim 12, wherein said glucosylceramide is a beta glucosylceramide (GC).

14. The composition according to claim 1, wherein said derivative of polyethoxylated castor oil is Cremophore EL (C:E).

15. The composition according to any one of claims 1 to 14, in a formulation adapted for add-on to a beverage, solid, semi-solid or liquid food, food additive, food supplement, medical food, botanical drug, drug and/or a pharmaceutical compound.

16. The composition according to claim 15, wherein said beverage and/or food comprise an increased content of sugar and/or alcohol and are optionally associated with increased blood sugar levels, altered insulin resistance and/or hepatic function.

17. The composition according to any one of claims 1 to 14 for controlling blood sugar levels in a subject, wherein said control is at least one of inhibiting increase or decrease in blood sugar levels, improving glucose tolerance or altering insulin resistance state.

18. The composition according to claim 17, for altering insulin resistance and/or hepatic function.

19. The composition according to claim 18 for use in the prevention or alleviation of symptoms related to a condition associated with at least one of, increased blood sugar levels, altered insulin resistance state and/or hepatic function, wherein said condition is any one of pre-diabetes, diabetes, obesity, hepatic disorder, pancreatic dysfunction, weight gain, alcohol intoxication, alcohol withdrawal and vertigo, any condition associated with alteration of pancreatic or liver function or tissue or organ damage, and drug-induced hepatic dysfunction.

20. The composition according to claim 19, for use in a method of prevention, alleviation or treatment of pre-diabetes or diabetes or any symptoms associated therewith in a subject in need thereof.

21. The pharmaceutical composition according to claim 18 for use in a method for treating a subject suffering from a disorder associated with altered insulin resistance and/or hepatic function.

22. The composition according to claim 21, wherein said disorder is any one of a hepatic disorder, pancreatic dysfunction, diabetes, obesity, insulin resistance, metabolic syndrome, alcohol intoxication, alcohol withdrawal and vertigo, an inflammation of pancreas, liver, muscle or the adipose tissue, inflammatory disorder and a malignancy.

23. The composition according to claims 1 to 14 for use in a method for treating, preventing, ameliorating, reducing or delaying the onset of an immune-related disorder, said composition comprising a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
at least one adjuvant selected from group of polyethylene glycol, polyethoxylated castor oil; beta cyclo dextrin or a derivative thereof; and
III. any combination of (a), (b) and (c) and optionally with any combination of (d), (e) and (g).

24. The composition according to claim 23, wherein said immune-related disorder is any one of an inflammatory disorder, an autoimmune disorder, an infectious disease and a proliferative disorder.

25. The composition according to any one of claims 1 to 14 for use in a method for treating liver damage and/or restoring liver function in a subject in need thereof, said composition comprising a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g).

26. The composition according to claim 25, wherein said subject is suffering from a liver disease, said liver disease is any one of viral, bacterial, fungal or parasitic liver disease, alcoholic or autoimmune hepatitis, alcoholic or autoimmune cirrhosis, alcoholic fatty liver disease, nonalcoholic fatty liver disease (NAFLD), liver steatosis, alcoholic or nonalcoholic steatohepatits (NASH), hepatocellular carcinoma, drug-induced liver disease and pediatric liver disease and metabolic liver disease.

27. The composition according to claim 25 for use in a method for treating, preventing, ameliorating, reducing or delaying the onset of acute or chronic toxic effect of a drug and for restoring liver function.

28. The composition according to claim 27, wherein said drug is an analgesic or an antipyretic drug.

29. A method for controlling at least one of blood sugar levels, altered insulin resistance and/or hepatic function, and treating an immune related disorder, treating liver damage, restoring liver function and for treating, preventing, ameliorating, reducing or delaying the onset of acute or chronic toxic effect of a drug on an organ or tissue, said method comprises providing to a subject:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g).

30. The method according to claim 29, wherein said sterol or a derivative or a mixture thereof is at least one plant stanols and sterol and any esters or mixtures thereof.

31. The method according to claim 30, wherein said plant stanols and sterol is Cardioaid™ comprising at least one of CardioAid-S, CardioAid-XF, CardioAid-SWD and CardioAid-SF.

32. The method according to claim 29, wherein said lunasin peptide or a derivative thereof is a peptide comprising the amino acid sequence as denoted y SEQ ID NO. 1, or any derivatives or functional fragments thereof.

33. The method according to claim 32, wherein said derivative of lunasin peptide is any one of Lunasin Reliv, LunaRichX and Relive.

34. The method according to claim 29, wherein said extract of a plant from the genus Moringa is an extract of Moringa oleifera.

35. The method according to claim 29, wherein said SE or any fraction thereof is at least one soy-derived polar and/or non-polar fraction.

36. The method according to claim 35, wherein said soy derived polar fraction comprises at least one of phospholipids, phosphatides or a combination thereof.

37. The method according to claim 36, wherein said phosphatides are any one of phosphatidylcholine (PC), phosphatidylinositol (PI) or a combination thereof, said polar fraction is designated M1.

38. The method according to claim 37, wherein said soy derived non-polar fraction comprises at least one of glycerides, phospholipids and phosphatides.

39. The method according to claim 38, wherein said at least one of glycerides, phospholipids and phosphatides, which are any one of phosphatidic acid (PA), phosphatidylethanolamine (PE) and phosphatidylcholine (PC), said non-polar fraction is designated OS.

40. The method according to claim 29, wherein said natural or synthetic beta-glycolipid is any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

41. The method according to claim 40, wherein said glucosylceramide is a beta glucosylceramide (GC).

42. The method according to claim 29, wherein said derivative of polyethoxylated castor oil is Cremophore EL (C:E).

43. The method according to claim 29, wherein said composition is provided in a formulation adapted for add-on to a beverage, solid, semi-solid or liquid food, food additive, food supplement, medical food, botanical drug, drug and/or a pharmaceutical compound.

44. The method according to claim 43, wherein said beverage and/or food comprises an increased content of sugar and/or alcohol.

45. The method according to claim 44, wherein said sugar and/or an alcohol is related to altered blood sugar levels, insulin resistance and/or hepatic function.

46. The method according to any one of claims 29 to 42 for controlling at least one of, blood sugar levels, altered insulin resistance and/or hepatic function.

47. The method according to claim 46, for the prevention or alleviation of symptoms related to a condition associated with altered blood sugar levels, insulin resistance and/or hepatic function, wherein said condition is any one of pre-diabetes, diabetes, obesity, hepatic disorder, pancreatic dysfunction, weight gain, alcohol intoxication, alcohol withdrawal and vertigo, any condition associated with alteration of pancreatic or liver function or tissue or organ damage, and drug-induced hepatic dysfunction.

48. The method according to claim 47, for prevention, alleviation or treatment of pre-diabetes or diabetes or any symptoms associated therewith in a subject in need thereof.

49. The method according to claim 47, for treating a subject suffering from a disorder associated with altered insulin resistance and/or hepatic function.

50. The method according to claim 49, wherein said disorder is any one of a hepatic disorder, pancreatic dysfunction, diabetes, obesity, insulin resistance, metabolic syndrome, alcohol intoxication, alcohol withdrawal and vertigo, an inflammation of pancreas, liver, muscle or the adipose tissue, inflammatory disorder and a malignancy.

51. The method according to any one of claims 29 to 42 for treating, preventing, ameliorating, reducing or delaying the onset of an immune-related disorder, said method comprising the step of administering a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g);
or any composition comprising the same.

52. The method according to any one of claims 29 to 42 for treating liver damage and/or restoring liver function in a subject in need thereof, said method comprising the step of administering a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g);
or any composition comprising the same.

53. The method according to claim 52, wherein said subject is suffering from a liver disease, said liver disease is any one of viral, bacterial, fungal or parasitic liver disease, alcoholic or autoimmune hepatitis, alcoholic or autoimmune cirrhosis, alcoholic fatty liver disease, nonalcoholic fatty liver disease (NAFLD), liver steatosis, alcoholic or nonalcoholic steatohepatits (NASH), hepatocellular carcinoma, drug-induced liver disease and pediatric liver disease and metabolic liver disease.

54. The method according to any one of claims 29 to 42 for treating, preventing, ameliorating, reducing or delaying the onset of acute or chronic toxic effect of a drug and for restoring liver function.

55. The method according to claim 54, wherein said drug is an analgesic or an antipyretic drug.

56. A soft or an alcoholic beverage or food comprising:
I. at least one of:
(a) at least one natural or synthetic sterol or a derivative or a mixture thereof;
(b) lunasin peptide or a derivative thereof; and
(c) at least one extract of a plant from the genus Moringa;
and at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g).

57. The soft or alcoholic beverage according to claim 56, wherein said sterol or a derivative or a mixture thereof is at least one plant stanols and sterol and any esters or mixtures thereof.

58. The soft or alcoholic beverage according to claim 56, wherein said plant stanols and sterol is Cardioaid™ comprising at least one of CardioAid-S, CardioAid-XF, CardioAid-SWD and CardioAid-SF.

59. The soft or alcoholic beverage according to claim 56, wherein said lunasin peptide or a derivative thereof is a peptide comprising the amino acid sequence as denoted by SEQ ID NO. 1, or any derivatives or functional fragments thereof.

60. The soft or alcoholic beverage according to claim 56, wherein said derivative of lunasin peptide is any one of Lunasin Reliv, LunaRichX and Relive.

61. The soft or alcoholic beverage according to claim 56, wherein said extract of a plant from the genus Moringa is an extract of Moringa oleifera.

62. The soft or alcoholic beverage according to claim 56, wherein said soy extract or any fraction thereof is soy-derived polar or non-polar fraction.

63. The soft or alcoholic beverage according to claim 62, wherein said fraction is a polar fraction, said phosphatides are any one of phosphatidylcholine (PC), phosphatidylinositol (PI) or a combination thereof, said polar fraction is designated M1.

64. The soft or alcoholic beverage according to claim 62, wherein said fraction is a non-polar fraction comprising at least one of glycerides, phospholipids and phosphatides, said non-polar fraction is designated OS.

65. The soft or alcoholic beverage according to claim 56, wherein said natural or synthetic beta-glycolipid is any one of a glucosylceramide, glycosphingolipid, monosaccharide ceramide, galatosylceremide, lactosylceramide, gal-gal-glucosyl-ceramide, GM2 ganglioside, GM3 ganglioside, globoside or any soy derivative or a combination thereof.

66. The soft or alcoholic beverage according to claim 56 wherein said glucosylceramide is a beta glucosylceramide (GC).

67. The soft or alcoholic beverage according to claim 56, wherein said derivative of polyethoxylated castor oil is Cremophore EL (C:E).

68. A pharmaceutical composition for use in a method for prevention of liver steatosis or liver disease in a healthy subject exposed to conditions inducing a liver disease, said composition comprising as an active ingredient a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g);
said composition optionally further comprising a pharmaceutically acceptable carrier.

69. A pharmaceutical composition for use in a method for prevention of diabetes in a subject with pre diabetic condition, said composition comprising as an active ingredient a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g);
said composition optionally further comprising a pharmaceutically acceptable carrier.

70. A method for enhancing and augmenting the therapeutic effect of at least one therapeutic agent in a subject treated with said at least one therapeutic agent, the method comprises providing to a subject a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g).

71. The method according to claim 70 wherein the compound/s or any combinations or compositions thereof as defined in any one of (I), (II) and (III), is administered concurrently or in parallel with the administration of said therapeutic agent.

72. A composition for use in enhancing and augmenting the therapeutic effect of at least one therapeutic agent in a subject treated with said at least one therapeutic agent, said composition comprising as an active ingredient a therapeutically effective amount of:
I. at least one of:
(a) at least one natural or synthetic sterol or a derivative or a mixture thereof;
(b) lunasin peptide or a derivative thereof; and
(c) at least one extract of a plant from the genus Moringa;
and optionally, at least one of:
II. at least one of:
(d) at least one soy extract (SE) or any fraction thereof;
(e) at least one natural or synthetic beta-glycolipid or any derivative thereof;
(f) at least one adjuvant selected from group of polyethylene glycol, polyethoxylated castor oil; beta cyclo dextrin or a derivative thereof; and
III. any combination of (a), (b) and (c) and optionally with any combination of (d), (e) and (g); said composition optionally further comprising a pharmaceutically acceptable carrier.
